(19) United States
(12) Patent Application Publication
(10) Pub. No.: US 2024/0366596 A1
(43) Pub. Date: Nov. 7, 2024

(54) TREATMENT OF HFPEF IN POST-MENOPAUSAL WOMEN WITH AN SGC STIMULATOR

(71) Applicant: Cyclerion Therapeutics, Inc., Cambridge, MA (US)

(72) Inventors: Robert S. Mittleman, Cambridge, MA (US); Albert Thomas Profy, Cambridge, MA (US); James E. Udelson, Cambridge, MA (US); Phebe Joseph Wilson, Raleigh, NC (US)

(21) Appl. No.: 18/292,747

(22) PCT Filed: Jul. 28, 2022
(86) PCT No.: PCT/US2022/038658

§ 371 Date: Jan. 26, 2024

Related U.S. Application Data
(60) Provisional application No. 63/301,245, filed on Jan. 20, 2022.
(60) Provisional application No. 63/232,417, filed on Aug. 12, 2021.
(60) Provisional application No. 63/226,226, filed on Jul. 28, 2021.

(51) Int. Cl. A61K 31/506; A61K 31/138; A61K 31/155; A61K 31/216; A61K 31/22; A61K 31/277; A61K 31/366; A61K 31/40; A61K 31/401; A61K 31/4025; A61K 31/41; A61K 31/4178; A61K 31/4375; A61K 31/4406; A61K 31/50; A61K 31/505; A61K 31/549; A61K 31/554; A61K 31/5575; A61K 31/5578; A61K 31/5585; A61K 31/585; A61K 31/7042; A61K 38/28; A61K 45/06; A61P 9/04

(52) U.S. Cl. CPC A61K 31/506; A61K 31/138; A61K 31/155; A61K 31/216; A61K 31/22; A61K 31/277; A61K 31/366; A61K 31/40; A61K 31/401; A61K 31/4025; A61K 31/41; A61K 31/4178; A61K 31/4375; A61K 31/4406; A61K 31/50; A61K 31/505; A61K 31/549; A61K 31/554; A61K 31/5575; A61K 31/5578; A61K 31/5585; A61K 31/585; A61K 31/7042; A61K 38/28; A61K 45/06; A61P 9/04

(57) ABSTRACT

The present disclosure relates to the use of stimulators of soluble guanylate cyclase (sGC), pharmaceutically acceptable salts thereof and pharmaceutical formulations or dosage forms comprising them, either alone or in combination with one or more additional therapeutic agents, for treating post-menopausal women with heart failure with preserved ejection fraction (HFpEF).

### RELATED APPLICATIONS

This application the benefit of the filing date, under 35 U.S.C. § 119 (e), to U.S. Provisional Application No. 63/226,226, filed on Jul. 28, 2021, U.S. Provisional Application No. 63/232,417, filed on Aug. 12, 2021, and U.S. Provisional Application No. 63/301,245, filed on Jan. 20, 2022. The entire contents of each of the foregoing applications are expressly incorporated herein by reference.

### FIELD OF THE INVENTION

The present disclosure relates to the use of stimulators of soluble guanylate cyclase (sGC), pharmaceutically acceptable salts thereof and pharmaceutical formulations or dosage forms comprising them, either alone or in combination with one or more additional therapeutic agents, for treating post-menopausal women with heart failure with preserved ejection fraction (HFpEF).

### BACKGROUND OF THE INVENTION

Heart Failure with Preserved Ejection Fraction (HFpEF)

Heart failure with preserved ejection fraction (HFpEF) is a significant source of morbidity and mortality in the United States (US) and globally. It currently comprises approximately 50% of new heart failure (HF) diagnoses in the US, and the prevalence is estimated to be at least 1% of the population, or more than 3 million Americans. Patients with HFpEF account for approximately half of the hospitalizations for HF and are frequently re-admitted following discharge. Mortality rates over 5 years for patients diagnosed with HFpEF have been reported to range from 55% to 74%. Patients with HFpEF have the signs and symptoms of HF, which may include dyspnea, orthopnea, lower extremity edema, pulmonary congestion, and cardiomegaly; they tend to have a low activity level, a suboptimal quality of life (QOL), and frequent episodes of depression.

In the last decades, HFpEF has been observed to increase in incidence over time, whereas the incidence of HF with reduced ejection fraction (HFrEF) appears to be declining (TSAO et al., Temporal trends in the incidence of and mortality associated with heart failure with preserved and reduced ejection fraction. JACC Heart Fail. 2018;6 (8): 678-85. doi: 10.1016/j.jchf.2018.03.006).

Although there are several approved treatments for HFrEF that reduce death and hospitalization rates, historically there have been very limited options for patients with HFpEF. In July of 2021, a Phase 3 study of Jardiance® (empagliflozin) in patients with chronic HF with HFpEF was announced to have met its primary endpoint (see https://beta.firstwordpharma.com/story/5343375; accessed 8 Jul. 2021). Top-line results from this EMPEROR-PRESERVED trial showed that the sodium-glucose transport protein 2 (SGLT2) inhibitor significantly reduced risk for the composite of cardiovascular death or hospitalization for HF compared with placebo. At the time of filing of this application, full results are pending and subsequent approval is expected. Also recently, an expanded US label for ENTRESTO® (sacubitril/valsartan) was approved to include reducing the risk of cardiovascular death and hospitalization for HF in adults with chronic HF (Highlights of prescribing information: ENTRESTO® [sacubitril and valsartan] tablets for oral use. East Hanover, NJ, Novartis Pharmaceuticals Corporation [2021]; https://beta.firstwordpharma.com/story/5343375; accessed 8 Jul. 2021). This decision made Entresto the first drug approved for use in both HFrEF and HFpEF. However, it is worth noting that the label expansion states that benefits are most evident in patients with left ventricular ejection fractions that are below normal.

Due to the lack of available therapies, treatment guidelines to date have been mostly limited to recommendations for controlling symptoms related to congestion, high blood pressure (BP), elements of ischemia, and management of risk factors and co-morbidities (YANCY C. W., et al. ACCF/AHA guideline for the management of heart failure: a report of the American College of Cardiology Foundation/American Heart Association Task Force on Practice Guidelines. Circulation. 2013; 128: e240-c327. doi: 10.1161/CIR.0b013e31829e8776). Renin-angiotensin-aldosterone system (RAAS) inhibitors, diuretics, calcium channel blockers, beta-blockers, diet, and exercise are the main recommendations for patients with HFpEF, although these interventions are not proven to reduce mortality in large randomized controlled trials (TSCHÖPE C., et al. Heart failure with preserved ejection fraction: current management and future strategies: Expert opinion on behalf of the Nucleus of the “Heart Failure Working Group” of the German Society of Cardiology [DKG]. Clin Res Cardiol. 2018 January; 107 [1]: 1-19. doi: 10.1007/s00392-017-1170-6).

It has been suggested that the lack of favorable effect of some of the therapies tested thus far may be due to the heterogeneity of the HFpEF patient population-rendering an approach that treats all patients using the same therapy overly simplistic (SHAH, et al. Circulation. 2016; 134:73-90. doi: 10.1161/CIRCULATIONAHA.116.021884).

There are differences between the epidemiology and the etiology of HFpEF and HFrEF. Patients with HFpEF tend to be older, more often female, and have fewer myocardial ischemic events, but display risk factors such as obesity, hypertension, and diabetes mellitus. Although there is hardly any difference between mortality and hospitalization rates of HFpEF and HFrEF, they do differ in their response to treatment with neuroendocrine antagonists. While solid evidence exists to support the therapeutic effects of treatment with RAAS blockers and beta-blockers in HFrEF, no such effects have been clearly demonstrated for these substance classes in patients with HFpEF.

It is becoming increasingly clear that the reason for the difference in treatment responses may be a lack of a full understanding of the pathophysiology of these diseases, including the use of different definitions and classifications, and their heterogenous presentations. Experts in the field are currently recommending that HFpEF be defined not as a solely cardiac disease, but as a systemic heterogeneous syndrome involving multiple organ systems (UPADHYA B., et al., Heart failure with preserved ejection fraction: New approaches to diagnosis and management. Clin Cardiol. 2020;43 (2): 145-155. doi: 10.1002/clc.23321).

### The NO-sGC-cGMP Pathway and sGC Stimulators

sGC is the primary receptor for nitric oxide (NO) in vivo. Upon binding to sGC, NO activates its catalytic domain and results in the conversion of guanosine-5′-triphosphate (GTP) into the secondary messenger cGMP. In turn, the increased level of cGMP modulates the activity of downstream effectors including protein kinases, phosphodiesterases (PDEs), and ion channels. In the body, NO is synthesized from arginine and oxygen by various nitric oxide synthase enzymes and by sequential reduction of inorganic nitrate. Experimental and clinical evidence indicates that reduced NO concentrations, reduced NO bioavailability, and/or reduced responsiveness to endogenously produced NO may contribute to the development of numerous diseases, including HF and other cardiovascular diseases (CVDs).

sGC stimulators are heme-dependent agonists of the sGC enzyme that work synergistically with varying amounts of NO to increase the enzymatic conversion of GTP to cGMP. sGC stimulators are clearly differentiated from and structurally unrelated to another class of NO-independent, heme-independent agonists of sGC known as sGC activators.

In addition, therapies that improve or restore the function of sGC offer considerable advantages over current alternative therapies that either target the NO-sGC-cGMP pathway or otherwise benefit from the upregulation of the NO-sGC-cGMP pathway by other means (e.g., NO donors, PDE5 inhibitors). There is an urgent need to develop new and safe therapies for patients with dysfunctional or downregulated NO-sGC-cGMP pathway signaling. Increased concentration of cGMP resulting from sGC stimulation leads to vasodilation, inhibition of platelet aggregation and adhesion, anti-hypertensive effects, anti-remodeling effects, anti-apoptotic effects, anti-inflammatory effects, anti-fibrotic effects, metabolic effects, and neuronal signal transmission effects. Compounds that stimulate sGC may also be useful in the prevention and/or treatment of diseases and disorders characterized by undesirable reduced bioavailability of and/or sensitivity to NO, such as those associated with conditions of oxidative stress or nitrosative stress. Thus, sGC stimulators are being studied to treat and/or prevent a range of diseases and disorders, such as HF (Neth Heart J. 2016; 24:268-74. doi 10.1007/s12471-016-0814-x).

### sGC Stimulators in HFpEF

Two sGC stimulators, namely praliciguat (Compound I-1 in this disclosure, Cyclerion Therapeutics) and vericiguat (Compound II-1 in this disclosure, Bayer-Merck), have been studied in clinical trials of HFpEF thus far and, in both cases, clinical development was halted due to a perceived lack of efficacy. Vericiguat was recently approved for the treatment of HFrEF (N Engl J Med. 2020; 382:1883-93. doi: 10.1056/NEJMoa1915928 and https://www.merck.com/news/merck-announces-u-s-fda-approval-of-verquvo-vericiguat/, accessed 8 Jul. 2021).

The SOCRATES-PRESERVED Phase 2b trial of vericiguat (PIESKE B., et al. Vericiguat in patients with worsening chronic heart failure and preserved ejection fraction: results of the soluble guanylate cyclase stimulator in heart failure patients with preserved EF [SOCRATES-PRESERVED] study. Eur Heart J. 2017;38 [15]: 1119-27. doi: 10.1093/eurheartj/chw593) randomized 477 participants with chronic HF and an ejection fraction (EF) of ≥45% (i.e., HFpEF) for 12 weeks of treatment with once-daily (QD) fixed doses of 1.25 or 2.5 mg vericiguat, doses of 5 or 10 mg vericiguat titrated from a 2.5-mg starting dose, or matching placebo. The two primary study endpoints were change from baseline in log-transformed N-terminal pro B-type natriuretic peptide (NT-proBNP) and left atrial volume at 12 weeks. Participants (48% women; mean age 73±10 years) were randomized within 4 weeks of HF hospitalization or of outpatient treatment with IV diuretics for HF. In the pooled three highest vericiguat dose arms, change in log NT-proBNP, and change in left atrial volume were NOT different from placebo.

Health status in the SOCRATES-PRESERVED study was assessed using the disease-specific Kansas City Cardiomyopathy Questionnaire (KCCQ) and the generic health-related quality of life measure EQ-5D (FILIPPATOS G., et al.; Patient-reported outcomes in the SOluble guanylate Cyclase stimulatoR in heArT failurE patientS with PRESERVED ejection fraction [SOCRATES-PRESERVED] study; Eur J Heart Failure. 2017; 19 (6), 782-91 doi: 10.1002/ejhf.800). Compared with the placebo arm, a larger proportion of participants treated with 10 mg vericiguat achieved clinically meaningful improvements in the KCCQ-clinical summary score (CSS; a combination of symptom and physical function domains).

Important domains of the KCCQ as well as EQ-5D scores demonstrated a dose-dependent relationship with vericiguat. In addition, improvements in KCCQ and EQ-5D scores paralleled improvement in physician-assessed New York Heart Association (NYHA) classification and clinical congestion. Therefore, it was concluded through these exploratory hypothesis-generating analyses that vericiguat was associated with clinically important improvements in participants' health status, as assessed by KCCQ and EQ-5D, and it was recommended that further studies be conducted to test the hypothesis that vericiguat improves physical functioning and health-related QOL in patients with HFpEF.

However, these results were not recapitulated in the Phase 2b VITALITY trial of vericiguat (CLARK K. A. A., and VELAZQUEZ E. J. Heart failure with preserved ejection fraction: time for a reset. JAMA. 2020;324 (15): 1506-08. doi: 10.1001/jama.2020.15566; and ARMSTRONG P. W., et al. Effect of vericiguat vs. placebo on quality of life in patients with heart failure and preserved ejection fraction—the VITALITY-HFpEF randomized clinical trial. JAMA. 2020;324 (15): 1512-21. doi: 10.1001/jama.2020.15922). In the VITALITY study, Armstrong and colleagues enrolled 789 participants with chronic HF, an LVEF of at least 45% (i.e., HFpEF), NYHA Class II to III symptoms within 6 months of a recent decompensation event (HF hospitalization or IV diuretics for HF without hospitalization), and elevated natriuretic peptides. Participants were randomized to receive vericiguat uptitrated to a daily dose of 15 mg (n=264) or 10 mg (n=263) or placebo (n=262). At 24 months, the primary outcome, change in physical performance as assessed by the mean change in the physical limitation score of the KCCQ (range, 0-100; higher scores indicate better health), was 5.5 points in the 15-mg group, 6.5 points in the 10-mg group, and 6.9 points in the placebo group. Differences between either vericiguat dose and placebo were NOT statistically significant.

Results of the initial data analysis for a Phase 2b trial of praliciguat in participants with HFpEF were also recently published (UDELSON J. E., et al. Effect of praliciguat on peak rate of oxygen consumption in patients with heart failure with preserved ejection fraction: the CAPACITY HFpEF randomized clinical trial. JAMA. 2020;324 [15]: 1522-31. doi: 10.1001/jama.2020.16641).

Given previously observed data, including both clinical and pre-clinical positive effects of praliciguat on metabolic parameters as well as preclinical positive effects in the heart, the CAPACITY HFpEF trial was designed to enrich for participants with HFpEF and concomitant cardiovascular risk factors who were expected to display maximal response to treatment (UDELSON J. E., et al. Rationale and design for a multicenter, randomized, double-blind, placebo-controlled, Phase 2 study evaluating the safety and efficacy of the soluble guanylate cyclase stimulator praliciguat over 12 weeks in patients with heart failure with preserved ejection fraction [CAPACITY HFpEF]. Am Heart J. 2020; 222:183-90. doi.org/10.1016/j.ahj.2020.01.009). Eligibility criteria required that participants had evidence in their medical history supporting clinical HF syndrome and evidence of established HF as assessed by more than 1 of 4 pre-selected criteria AND that they met at least 2 of the following criteria at the Screening Visit: a. diagnosis of type 2 diabetes mellitus or prediabetes (currently treated or hemoglobin A1c≥5.6); b. history of hypertension (taking at least 1 antihypertensive medication or had current seated blood pressure [BP]>140/90 mmHg); c. body mass index (BMI)>30 kg/m²; and d. age was ≥70 years. The study enrolled both male and female participants.

A total of 143 of the 155 participants who completed the trial were included in the prespecified Primary Analytic Population, which was defined as those participants who had taken the assigned study drug regimen for at least 8 of the 12 treatment weeks, had ≥1 evaluable post-baseline assessment(s), and did not have any major protocol deviations. In the placebo (n=78) and praliciguat (n=65) groups of this population, changes from baseline peak oxygen consumption (VO₂) at Week 12 were 0.04 mL/kg/min (95% CI, −0.49 to 0.56) and −0.26 mL/kg/min (95% CI, −0.83 to 0.31), respectively; the placebo-adjusted least-squares mean (LSM) between-group difference in change from baseline was −0.30 mL/kg/min (95% CI, −0.95 to 0.35; P=0.37).

In addition, none of the results for the 3 prespecified secondary endpoints were statistically significant when the Primary Analytic Population was analyzed. In the placebo and praliciguat groups, changes in 6-minute walk test (6MWT) distance were 58.1 meters (m) (95% CI, 26.1-90.1) and 41.4 m (95% CI, 8.2-74.5), respectively; the placebo-adjusted LSM between-group difference in change from baseline was −16.7 m (95% CI, −47.4 to 13.9). In the placebo and praliciguat groups, the placebo-adjusted LSM between-group difference in change in ventilation/carbon dioxide production slope was −0.3 (95% CI, −1.6 to 1.0). Compared with the placebo group, more participants in the praliciguat group had at least one treatment-emergent adverse event (TEAE) of dizziness (9.9% vs. 1.1%), hypotension (8.8% vs. 0%), and/or headache (11% vs. 6.7%). The percentage of participants with at least one serious TEAEs was similar between the groups (10% in the praliciguat group vs. 11% in the placebo group).

Thus, among participants in the CAPACITY HFpEF trial, the sGC stimulator praliciguat did not significantly improve peak VO₂ from baseline to Week 12 or significantly improve any of the prespecified secondary endpoints compared with placebo. As a result, it was initially concluded that these findings did not support the use of praliciguat in patients with HFpEF, and the development of praliciguat for HFpEF was halted.

### HFpEF and the NO-sGC-cGMP Pathway in Women

In developed countries, cardiovascular diseases (CVDs) are the leading cause of death, regardless of sex (UEDA K., et al. Regulatory actions of estrogen receptor signaling in the cardiovascular system. Front. Endocrinol. 2020; 10:909. doi: 10.3389/fendo.2019.00909). The incidence of CVD is higher in men before the age of 50 than in pre-menopausal women. However, after menopause, CVD incidence in women increases and eventually exceeds the CVD incidence in men. Furthermore, men more often die of ischemic heart disease, while women more often die from stroke and HF. As women are less likely to develop CVD before menopause, the endogenous female hormone estrogen appears to provide protection against CVD. Studies with hormone replacement therapy (HRT) appear to confirm these findings, in particular when HRT is initiated no later than 10 years after menopause (HODIS H N, et al. Vascular effects of early versus late postmenopausal treatment with estradiol. N Engl J Med. (2016) 374:1221-31. doi: 10.1056/NEJMoa1505241).

The prevalence of overall HF is about 2.6 million women and 3.1 million men in the US, with a higher prevalence in advanced age. In both sexes, the incidence of HFpEF and HFrEF increases with age; however, HFrEF incidence is higher in men at any given age, whereas HFpEF incidence is higher in women at any given age, and particularly after menopause. With respect to HFpEF, it is known that the disease is accompanied by a diversity of risk factors and comorbidities, including hypertension, obesity, and diabetes mellitus. Aging and being a woman are two of the most important, and appear to be independent of other risk factors.

At diagnosis, women with HF tend to be older than men and exhibit a different phenotype. Moreover, despite the fact that women present with HF at an older age, when comorbidities are more frequent, some studies show that women have a lower cardiovascular (CV) and all-cause mortality, suggesting that the phenotypic differences in HF presentation and prognosis between women and men may be the consequence of progressive, sex-specific changes in CV physiology. Furthermore, the menopausal transition may influence the development of CV risk factors in women. In addition, different underlying processes could be responsible for the potential differences in drug tolerance and toxicity to therapeutic interventions that have been documented in women (TAMARGO J., et al. Gender differences in the effects of cardiovascular drugs. Eur Heart J Cardiovasc Pharmacother. 2017;3 [3]: 163-82 REVIEW. doi: 10.1093/chjcvp/pvw042).

It has been suggested that pre-menopausal women exert some of the observed estrogen-mediated cardio-protection effects through the NO/sGC/cGMP pathway (SICKINGHE A. A., et al. Estrogen contributions to microvascular dysfunction evolving to heart failure with preserved ejection fraction. Front Endocrinol. 2019; 10:442. doi: 10.3389/fendo.2019.0044). After menopause, women lack this protection, and are thus at an increased risk for CVD in general, and HF in particular. HFpEF is the dominant form of HF in post-menopausal women (>60% prevalence). Thus, the increasing prevalence of HFpEF in the general population, and particularly in women who are post-menopausal, is an important unmet need.

Given the limited treatment options, there remains significant and urgent unmet medical need for new treatments for patients with HFpEF. In particular, there is significant unmet need for HFpEF treatments for women, who represent the majority of patients with HFpEF and tend to present with the disease after menopause. Despite the fact that they represent the majority of patients with HFpEF, women have historically been underrepresented in clinical trials of the disease (XURUIN J, Women's participation in cardiovascular clinical trials from 2010 to 2017; Circulation. 2020; 141:540-48. doi/10.1161/CIRCULATIONAHA.119.043594) and there is a need to develop treatments for HFpEF that address the characteristics and clinical presentation of female patients. Increasing cGMP through stimulation of the NO-sGC-cGMP pathway is a promising therapeutic target for HFpEF patients, particularly in women.

### SUMMARY OF THE INVENTION

This disclosure is based on the surprising results obtained from a post-hoc sub-population analysis of the data obtained from the CAPACITY HFpEF trial of praliciguat. This post-hoc analysis unexpectedly demonstrated positive effects in elderly (i.e., older than 70 years) women that were not observed in male participants. We hypothesize for the reasons summarized in the Detailed Description of the Invention section that these effects not only apply to praliciguat, but that they also extend to other sGC stimulators and to the selected subpopulation of post-menopausal women with HFpEF.

In a first aspect, the present invention is directed to a method of treating post-menopausal women with HFpEF comprising administering to said women, alone or in combination therapy, a therapeutically effective amount of an sGC stimulator or a pharmaceutically acceptable salt thereof.

In a second aspect, the present invention is directed to a method of treating post-menopausal women with HFpEF comprising administering to said women, alone or in combination therapy, a pharmaceutical composition or dosage form comprising a therapeutically effective amount of an sGC stimulator or a pharmaceutically acceptable salt thereof.

In a third aspect, the present invention is directed to a method of improving exercise capacity in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a fourth aspect, the present invention is directed to a method of improving exercise tolerance in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a fifth aspect, the present invention is directed to a method of improving functional capacity (i.e., the ability of a person to function with respect to the activities of daily living) in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a sixth aspect, the present invention is directed to a method of improving heart function in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a seventh aspect, the present invention is directed to a method of improving health status in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In an eighth aspect, the present invention is directed to a method of improving quality of life (QOL) in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In an ninth aspect, the present invention is directed to a method of delaying clinical worsening in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a tenth aspect, the present invention is directed to a method of reducing the risk of hospitalization or re-hospitalization for HF in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In an eleventh aspect, the present invention is directed to a method of reducing the risk of cardiovascular death or all-cause mortality in post-menopausal women with HFpEF comprising administering to said women, alone or in combination therapy, a therapeutically effective amount of an sGC stimulator or a pharmaceutically acceptable salt thereof, or a pharmaceutical composition or dosage form comprising a therapeutically effective amount of an sGC stimulator or a pharmaceutically acceptable salt thereof.

In a twelfth aspect, the invention is further directed to the use of an sGC stimulator or a pharmaceutically acceptable salt thereof, or a pharmaceutical composition or a dosage form comprising an sGC stimulator or a pharmaceutically acceptable salt thereof, for the manufacture of a medicament for the treatment of post-menopausal women with HFpEF; or for the manufacture of a medicament for the improvement of exercise capacity, exercise tolerance, functional capacity, heart function, health status, or quality of life (QOL) in post-menopausal women with HFpEF; or for the manufacture of a medicament for delaying the clinical worsening in post-menopausal women with HFpEF; or for the manufacture of a medicament for reducing the risk of hospitalization or re-hospitalization due to HF or the risk of cardiovascular death or all-cause mortality in post-menopausal women with HFpEF.

In a thirteenth aspect, the invention is further directed to an sGC stimulator, or a pharmaceutically acceptable salt thereof, a pharmaceutical composition or a dosage form comprising the sGC stimulator or a pharmaceutically acceptable salt thereof for use in the treatment of post-menopausal women with HFpEF; or for use in improving exercise capacity, exercise tolerance, functional capacity, heart function, health status, or quality of life (QOL) in post-menopausal women with HFpEF; or for use in delaying clinical worsening in post-menopausal women with HFpEF; or for use in reducing the risk of hospitalization or re-hospitalization due to HF or the risk of cardiovascular death or all-cause mortality in post-menopausal women with HFpEF.

In certain embodiments of the above first to thirteenth aspects, women with HFpEF are elderly post-menopausal women. In some of these embodiments, elderly post-menopausal women are those older than 60, older than 65, older than 70, or older than 75 years old.

### BRIEF DESCRIPTION OF THE FIGURES

FIG. 1 shows change from baseline in peak VO₂ for women age 70 or older who received 40 mg praliciguat compared to those who received placebo. The left graph shows LS Mean Change from Baseline in Peak VO₂ at Week 8 and Week 12 and the right graph shows LSMean Difference vs. Placebo.

FIG. 2 shows sensitivity analyses of the primary efficacy endpoint in the sub-population of women with age >=70.

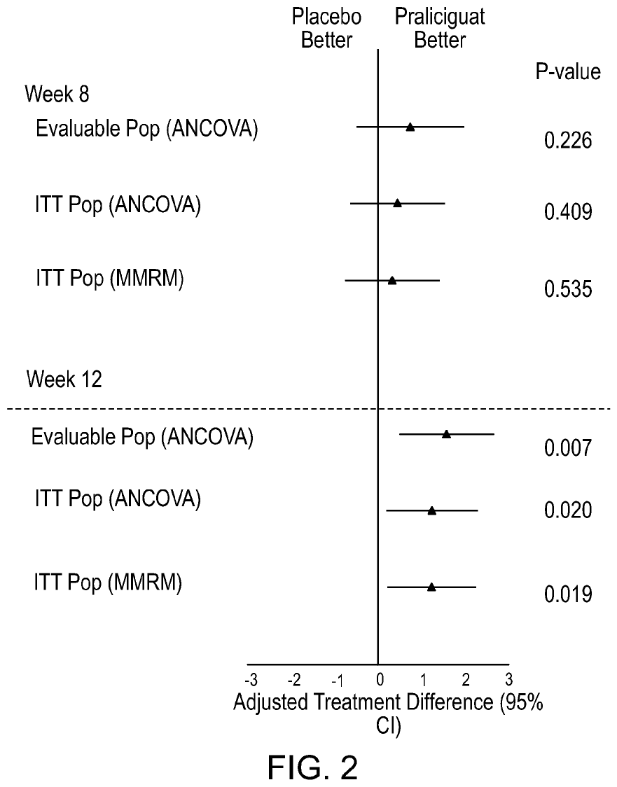

FIG. 3 shows change from baseline in peak VO₂ for individual women ages >=70 treated with 40 mg praliciguat (on the left) compared to placebo (on the right), where lines represent change from baseline to week 12 and are color coded by green indicating an increase, and black indicating a decrease.

FIG. 4 shows effect of olinciguat (oli 10 mg/kg, OLI10) on cardiac weights, lung weights, and NT-proBNP levels in the Dahl salt-sensitive rat model. Graph A shows weights of left-ventricular wall plus ventricular septum normalized to body weight. Graph B shows lung weights normalized to body weights. Graph C shows plasma NT-proBNP levels. All data are plotted as mean±SEM. **p<0.01. ***p<0.001 vs high-salt (HS) control.

FIG. 5 shows change from baseline in NT-proBNP for animals treated with enalapril (ENP), enalapril+10 mg/kg olinciguat (ENP+OLI10) and enalapril+30 mg/kg olinciguat (ENP+OLI30 groups) in the ZSF1 rat model of diabetic nephropathy with metabolic syndrome. All data are plotted as mean±SEM. #p<0.05. ##p<0.01 vs obese control.

FIG. 6 shows change from baseline to week 12 in peak VO₂ for women who received 40 mg praliciguat compared to those who received placebo. Left panel: full study population; right panel: elderly women >70 years.

FIG. 7 shows change from baseline to week 12 in peak VO₂ for women >70 years who received 40 mg praliciguat compared to those who received placebo. Left panel: subpopulation with a higher than 1 mL/O₂/kg/min improvement in peak VO₂ at 12 weeks of treatment; right panel: subpopulation with a higher than 1.5 mL/O₂/kg/min improvement in peak VO₂ at 12 weeks of treatment.

### DETAILED DESCRIPTION OF THE INVENTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications, and equivalents that may be included within the scope of the present invention as defined by the claims. The present invention is not limited to the methods and materials described herein but includes any methods and materials similar or equivalent to those described herein that could be used in the practice of the present invention. In the event that one or more of the incorporated literature references, patents, or similar materials differ from or contradict this application, including but not limited to defined terms, term usage, described techniques or the like, this application controls.

### Definitions and General Terminology

As used herein, the terms “subject” and “patient” are used interchangeably. A subject or a patient is a human subject or human patient. A subject or a patient that participates in a clinical trial and is dosed with either active drug or placebo is a “participant” in the clinical trial. In one embodiment, the patient is a post-menopausal woman treated or to be treated with an sGC stimulator or a pharmaceutically acceptable salt thereof described herein.

For the terms “for example” and “such as,” and grammatical equivalences thereof, the phrase “without limitation” or “and without limitation” is understood to follow unless explicitly stated otherwise.

The terms “administer”, “administering” or “administration” in reference to a compound or pharmaceutical agent are understood to mean introduction of the compound or pharmaceutical agent into the body of the patient who is in need of treatment. When a compound of the invention is used in combination with one or more other therapeutic agents, “administration” and its variants are each understood to encompass concurrent and/or sequential introduction of the compound of the invention and the other therapeutic agents into the patient.

“Treat”, “treating” or “treatment” with regard to a disorder, disease, condition, symptom or syndrome, refers to abrogating or improving the cause and/or the effects (i.e., the symptoms, physiological, physical, psychological, emotional or any other clinical manifestations, observations or measurements, or improving pathological assessments) associated with the disorder, disease, condition, or syndrome. As used herein, the terms “treat”, “treatment” and “treating” also refer to the delay, reduction, prevention, or amelioration of the progression (i.e., the known or expected progression of the disease), severity, and/or duration of the disease, or the delay, reduction, prevention, or amelioration of the progression of one or more symptoms (i.e., “managing” without “curing” the condition), resulting from the administration of one or more therapies.

In specific embodiments, the terms “treat”; “treatment” and “treating” refer to the amelioration of at least one measurable symptom, physiological, physical, psychological, emotional or any other clinical manifestations, observations or measurements, or improving pathological assessments associated with HFpEF (e.g., peak VO₂, exercise capacity, functional capacity, or heart function).

In other embodiments, the terms “treat”. “treatment” and “treating” refer to the inhibition of the progression of HFpEF, either physically by, e.g., stabilization of at least one clinically discernible parameter (e.g., peak VO₂, 6-minute walk distance), or stabilizing the disease progression (e.g., reducing the risk of future hospitalizations or future cardiovascular death or all-cause mortality).

According to the National Heart, Lung and Blood Institute, heart failure (HF) is a condition in which the heart cannot pump enough blood to meet the body's needs. In some cases, the heart cannot fill with enough blood. In other cases, the heart cannot pump blood to the rest of the body with enough force. Some people have both problems. HF develops over time as the heart's pumping action grows weaker. The condition can affect the right side or the left side of the heart only, or it can affect both sides of the heart. Most cases involve both sides of the heart.

Right-side HF occurs if the heart cannot pump enough blood to the lungs to pick up oxygen. Left-side HF occurs if the heart cannot pump enough oxygen-rich blood to the rest of the body. Right-side HF may cause fluid to build up in the feet, ankles, legs, liver, abdomen, and the veins in the neck. Right-side and left-side HF also may cause shortness of breath and fatigue.

The leading causes of HF are diseases that damage the heart. Examples include ischemic heart disease, high blood pressure, and diabetes.

“Ejection fraction” (EF) is an important measurement in the diagnosis and surveillance of HF. However, it is possible for patients with normal EF to have HF, and this form of the disease, HFpEF, also known as diastolic HF, is becoming increasingly prevalent.

In HFpEF, the muscles of the heart contract normally and the heart may seem to pump a normal proportion of the blood that enters it. However, heart muscle thickening may cause the ventricle to hold an abnormally small volume of blood. Therefore, although the heart's output may still appear to be in the normal range, its limited capacity is inadequate to meet the body's requirements.

In HFrEF, also known as systolic HF, the heart muscle is not able to contract adequately and, therefore, expels less oxygen-rich blood into the body. Patients with this form of the disease have lower-than-normal “left ventricular ejection fraction (LVEF)” on an echocardiogram. As used herein, EF refers to LVEF and the two terms are interchangeable.

According to the National Heart, Lung and Blood Institute, an LVEF of 50% to 75% indicates a normal pumping ability (preserved EF), whereas a range of 36% to 49% is considered below normal, and 35% or lower is considered low pumping ability (reduced EF). Fatigue and shortness of breath are common symptoms of both HFpEF and HFrEF.

According to the European Society of Cardiology, patients with EF between 40% and 55% are defined as having HF with mid-range EF. Most HFpEF trials require at least LVEF≥45% or at least LVEF≥40%.

Throughout this disclosure, a patient is considered as having “established HF” if, in addition to evidence of a medical history of HF, the patient presents with at least one clinical manifestation selected from the group consisting of: prior well-documented HF hospitalization, elevated B-type natriuretic peptide (BNP) and/or N-terminal pro B-type natriuretic peptide (NT-proBNP) levels, echocardiographic evidence of structural changes consistent with HFpEF, and hemodynamic evidence of elevated filling pressures.

As used herein, a patient with HFpEF is considered to have an “elevated level of BNP” if the concentration of BNP is ≥100 pg/mL (for patients with sinus rhythm) or ≥200 pg/mL (for patients with atrial fibrillation).

As used herein, a patient with HFpEF is considered to have an “elevated level of NT-proBNP” if the concentration of NT-proBNP is ≥300 pg/mL (for patients with sinus rhythm) or ≥600 pg/mL (for patients with atrial fibrillation).

As used herein, a patient is considered as having “established HFpEF” if, in addition to a medical history of HF, echocardiographic evidence of structural changes consistent with HFpEF and/or hemodynamic evidence of elevated filling pressures are present.

It is noteworthy that an elevated BNP and/or NT-proBNP level was a sufficient but not a necessary requirement to be considered as having established HF to be eligible for enrollment in the clinical trial described in the Examples section (CAPACITY HFpEF of praliciguat), in contrast to what was required in many other clinical trials for HFpEF. This approach was put into place based on evidence that some patients with normal BNP and or NT-proBNP levels at rest can have elevated filling pressures at rest or with exercise (SHEA C., et al., Treatment with the soluble guanylate cyclase stimulator IW-1973 reduces N-terminal pro B-type natriuretic peptide and improves heart function in rat models of heart failure with preserved ejection fraction and heart failure with reduced ejection fraction. The FASEB Journal 2017;31 [S1]: 676.2; doi. 10.1096/fascbj.31.1 supplement.676.2; and ANJAN V. Y., et al. Prevalence, clinical phenotype, and outcomes associated with normal B-type natriuretic peptide levels in heart failure with preserved ejection fraction. Am J Cardiol. 2012 Scp 15;110 [6]: 870-6; doi: 10.1016/j.amjcard.2012.05.014), and based on the fact that patients with the obesity/metabolic phenotype of HFpEF who were enrolled in the CAPACITY HFpEF trial were expected to have a low BNP due to the known effects of obesity on increasing BNP clearance and reducing BNP production (OBOKATA M., et al. Evidence supporting the existence of a distinct obese phenotype of heart failure with preserved ejection fraction. Circulation. 2017 Jul. 4; 136 [1]: 6-19; doi: 10.1161/CIRCULATIONAHA.116.026807).

As used herein, patients with “limited exercise capacity” are defined as those who display a mean peak oxygen consumption (VO₂) measurement of less than 80% of the age-and sex-adjusted normal (FLETCHER G. F., et al. Exercise standards. A statement for healthcare professionals from the American Heart Association. Writing Group. Circulation. 1995 Jan. 15;91 (2): 580-615; see Table 2 in Examples section) as measured by cardiopulmonary exercise testing (CPET). To ensure an adequate exercise test, a respiratory exchange ratio (RER)≥1.0 must also be achieved. Limited exercise capacity or “exercise intolerance” is the primary symptomatic manifestation of HFpEF and is associated with a poor quality of life and increased mortality. Peak VO₂ is an objective measure of exercise and functional capacity and represents the integrated response of multiple aspects of the cardiovascular, pulmonary, and skeletal muscle systems. In patients with HFpEF, peak VO₂ has been responsive to interventions such as exercise training, caloric restriction, and aldosterone antagonism (the latter only in highly selected patients).

As used herein, a “metabolic/inflammatory phenotype” is defined as that of patients or clinical trial participants who display two or more risk factors for developing HFpEF independently selected from the group consisting of: diabetes or prediabetes (e.g., type 2 diabetes mellitus or prediabetes), hypertension, obesity/being overweight, and older age (≥70 years old). This metabolic/inflammatory phenotype includes patients/participants who display the metabolic cardiovascular pathology described by Paulus et al and Shah et al, within the broader HFpEF population (SHAH S. J., et al. Phenotype-specific treatment of heart failure with preserved ejection fraction: a multiorgan roadmap. Circulation. 2016;134 [1]: 73-90; and PAULUS W. J., TSCHOPE C. A novel paradigm for heart failure with preserved ejection fraction: comorbidities drive myocardial dysfunction and remodeling through coronary microvascular endothelial inflammation. J Am Coll Cardiol. 2013;62 [4]: 263-71).

As used herein, “insulin sensitivity” refers to how sensitive the body is to the effects of insulin. In a clinical setting, insulin sensitivity may be determined using homeostatic model assessment of insulin resistance (HOMA-IR), which is a method for assessing β-cell function and insulin resistance from basal (fasting) glucose and insulin or C-peptide concentrations. The normal HOMA-IR value for a healthy human ranges from 0.5 to 1.4. A HOMA-IR value <1.0 means that the person is insulin-sensitive, which is optimal. A value >1.9 is indicative of early “insulin resistance.” A level greater than 2.9 is indicative of elevated “insulin resistance.” In other settings, a medical practitioner may determine if a person is insulin resistant, by the presence of a combination of metabolic syndrome traits. In some embodiments, metabolic syndrome traits can be independently selected from: overweight-obesity, elevated triglycerides, elevated HDL, elevated fasting insulin, insulin-to-glucose ratio, elevated glucose levels, elevated HbA1c and hypertension.

### Therapeutic Methods

This disclosure is based on the surprising results obtained from an exploratory, post-hoc sub-population analysis of the data obtained from the CAPACITY HFpEF trial of praliciguat. This post-hoc analysis unexpectedly demonstrated positive effects in elderly women (older than 70 years) that were not observed in male participants. We hypothesize herein that these effects may not only apply to praliciguat, but that they will also extend to other sGC stimulators, when the stimulator is administered to a selected subpopulation of post-menopausal women with HFpEF. It is plausible that the reason that these positive effects have not been observed thus far in this population in trials involving the NO-sGC-cGMP pathway is that, as noted above, women have usually been underrepresented in many trials of HFpEF, and their representation has not paralleled the prevalence of the disease in this population. This sub-analysis by a combination of age and gender from the CAPACITY HFpEF trial herein disclosed, represents the first confirmation that sGC stimulators can provide benefit to post-menopausal women with HFpEF.

As noted above, in the Background section, the estrogen and NO-sGC-cGMP pathways are interlinked, so it is highly plausible that increases in cGMP synthesis by sGC stimulation in post-menopausal women, as observed in the CAPACITY trial, could partially be mimicking the known estrogen-mediated cardio-protection effects in pre-menopausal women, and this effect should extend to any sGC stimulator, not just praliciguat.

Related literature published in the field of HF may help further support, mechanistically, the hypothesis that this treatment effect in post-menopausal women using sGC stimulators should also extend to other sGC stimulators, in addition to praliciguat. For instance, the signaling pathways affected by ARNIs and MRAs are also believed to be interlinked with the estrogen pathway and also converge on with the NO-sGC-cGMP pathway (PARK, M., et al. cGMP at the centre of attention: emerging strategies for activing the cardioprotective PKG pathway. Basic Res Cardiol (2018) 113:24. doi.org/10.1007/s00395-018-0679-9 and LA BIWER et al., Mineralocorticoid and Estrogen Receptors in Endothelial Cells Coordinately Regulate Microvascular Function in Obese Female Mice, Hypertension. 2021; 77:2117-2125. DOI: 10.1161/HYPERTENSIONAHA.120.16911.)

The angiotensin receptor 1 antagonist/neprilysin inhibitor (ARNI) sacubitril-valsartan has been observed to have differential effects in women versus men, as suggested in the prospective comparison of ARNI with ARB global outcomes in the PARAGON-HF trial in which a prespecified subgroup analysis by sex was carried out (MCMURRAY, J. J. V, et al. Effects of sacubitril-valsartan vs. valsartan in women compared with men with heart failure and preserved ejection fraction. Circulation. 2020; 141:338-51. doi: 10.1161/CIRCULATIONAHA.119.044491). The primary outcome was a composite of first and recurrent hospitalizations for HF and death from cardiovascular causes. Secondary efficacy and safety outcomes were also reported. Overall, 2479 women (51.7%) and 2317 men (48.3%) were randomized. Women were older and had more obesity, less coronary disease, and lower estimated glomerular filtration (eGRF) and NT-proBNP levels than men. For the primary study outcome, the rate ratio for sacubitril-valsartan versus valsartan was 0.73 (95% CI, 0.59-0.90) in women and 1.03 (95% CI, 0.84-1.25) in men (P_{interaction}=0.017). The benefit from sacubitril-valsartan was attributable to reduction in HF hospitalization. Thus, as compared with valsartan, sacubitril-valsartan seemed to reduce the risk of HF hospitalization more in women than in men. Although the possible sex-related modification of the effect of treatment has several potential explanations, the authors of the study did not provide a definite mechanistic basis for this finding.

Positive effects on women and not men were also observed in an exploratory, post-hoc, non-prespecified analysis of the treatment of preserved cardiac function heart failure with an aldosterone antagonist trial (TOPCAT) trial, in which a mineralocorticoid receptor antagonist (MRA) was studied vs. placebo (JACC Heart Fail. 2019 March;7 (3): 228-38. doi: 10.1016/j.jchf.2019.01.003:10.1016/j.jchf.2019.01.003). Subjects with symptomatic HF and an LVEF≥45% were randomized to the MRA spironolactone or placebo. Subjects enrolled from the Americas were analyzed. The primary outcome was a composite of cardiovascular (CV) death, cardiac arrest, or HF hospitalization. Secondary outcomes included all-cause, CV, and non-CV mortality, and CV, HF, and non-CV hospitalization. Sex differences in outcomes and treatment effect were determined using time-to-event analysis. In total, 882/1767 (49.9%) subjects were women. Women were older with fewer comorbidities, but worse patient-reported outcomes compared with men. There were no sex differences in outcomes in the placebo arm or in response to spironolactone for the primary outcome or its components. However, spironolactone was associated with reduced all-cause mortality in women (HR 0.66, p=0.01), but not in men (p_{interaction}=0.02). The authors suggested that prospective evaluation is needed to determine whether spironolactone may be effective for treatment of HFpEF in women.

In a first aspect, the present invention is directed to a method of treating post-menopausal women with HFpEF comprising administering to said women, alone or in combination therapy, a therapeutically effective amount of an sGC stimulator or a pharmaceutically acceptable salt thereof.

In a second aspect, the present invention is directed to a method of treating post-menopausal women with HFpEF comprising administering to said women, alone or in combination therapy, a pharmaceutical composition or dosage form comprising a therapeutically effective amount of an sGC stimulator or a pharmaceutically acceptable salt thereof.

In a third aspect, the present invention is directed to a method of improving exercise capacity in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a fourth aspect, the present invention is directed to a method of improving exercise tolerance in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a fifth aspect, the present invention is directed to a method of improving functional capacity (i.e., the ability of a person to function with respect to the activities of daily living) in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a sixth aspect, the present invention is directed to a method of improving heart function in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a seventh aspect, the present invention is directed to a method of improving health status in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In an eighth aspect, the present invention is directed to a method of improving quality of life in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a ninth aspect, the present invention is directed to a method of delaying clinical worsening in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a tenth aspect, the present invention is directed to a method of reducing the risk of hospitalization or re-hospitalization for HF in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In an eleventh aspect, the present invention is directed to a method of reducing the risk of cardiovascular death or all-cause mortality in post-menopausal women with HFpEF comprising administering to said women, alone or in combination therapy, a therapeutically effective amount of an sGC stimulator or pharmaceutically acceptable salt thereof, or a pharmaceutical composition or dosage form comprising a therapeutically effective amount of an sGC stimulator or a pharmaceutically acceptable salt thereof.

In a twelfth aspect, the invention is further directed to the use of an sGC stimulator or a pharmaceutically acceptable salt thereof, or a pharmaceutical composition or a dosage form comprising an sGC stimulator or a pharmaceutically acceptable salt thereof, for the manufacture of a medicament for the treatment post-menopausal women with HFpEF; or for the for the manufacture of a medicament for the improvement of exercise capacity, exercise tolerance, functional capacity, heart function, health status or quality of life in post-menopausal women with HFpEF; or for the manufacture of a medicament for delaying clinical worsening in post-menopausal women with HFpEF; or for the manufacture of a medicament for reducing the risk of hospitalization or re-hospitalization due to HF or the risk of cardiovascular death or all cause mortality in post-menopausal women with HFpEF.

In a thirteenth aspect, the invention is further directed to an sGC stimulator, or a pharmaceutically acceptable salt thereof, a pharmaceutical composition or a dosage form comprising the sGC stimulator or a pharmaceutically acceptable salt thereof for use in the treatment of post-menopausal women with HFpEF; or for use in improving exercise capacity, exercise tolerance, functional capacity, heart function, health status or quality of life in post-menopausal women with HFpEF; or for use in delaying clinical worsening in post-menopausal women with HFpEF; or for use in reducing the risk of hospitalization or re-hospitalization due to HF or the risk of cardiovascular death or all cause mortality in post-menopausal women with HFpEF.

In certain embodiments of the first to thirteenth aspects, women with HFpEF are elderly post-menopausal women. In some of these embodiments, elderly post-menopausal women are those older than 60, older than 65, older than 70, or older than 75 years old. In some embodiments of the first to thirteenth aspects, the patient is older than 70 years old at the start of treatment. In other embodiments the patient is younger than 70 years old at the start of treatment. In still other embodiments, the patient is 70 years old at the start of treatment.

In some embodiments of the first to thirteenth aspects, the patient with HFpEF being treated has LVEF>40%. In other embodiments, the patient with HFpEF being treated with the methods of the present invention has LVEF>45%. In still other embodiments, EF>50%. In some embodiments of the first to thirteenth aspects, the patient has an EF≥40%. In some embodiments of the first to thirteenth aspects, the patient has an EF≥45%. In still other aspects the patient has an EF≥50%.

In certain embodiments of the first to thirteenth aspects, the patient with HFpEF has had a hospitalization or emergency department visit for HF within the year prior to the start of treatment.

In certain embodiments of the first to thirteenth aspects, the patient with HFpEF displays a volume overload on presentation, as evidenced by at least 2 of the following signs: jugular venous distension, pitting edema ≥1+, ascites, pulmonary, congestion on chest x-ray, or pulmonary rales.

In certain embodiments of the first to thirteenth aspects, the patient with HFpEF has an elevated level of BNP. In certain embodiments of the first to thirteenth aspects, the patient with HFpEF has an elevated level of NT-proBNP. In certain embodiments, the patient has elevated levels of BNP and NT-proBNP. In some embodiments, these elevated values have been displayed by the patient at least within the last 6 months before the start of treatment. In other embodiments, the patient does not present elevated levels of BNP or NT-proBNP at the start of treatment.

In certain embodiments of the first to thirteenth aspects, the patient with HFpEF displays a clinical response when treated with intravenous (IV) diuretics. In some embodiments, a clinical response when treated with IV diuretics is characterized by a reduction in weight accompanied with reduction of symptoms after diuresis. In other embodiments, it is characterized by an improvement in shortness of breath (orthopnea or dyspnea) after diuresis. In other embodiments, it is characterized by an improvement in blood oxygenation after diuresis (as assessed for example by pulse oximetry). In certain embodiments of the first to thirteenth aspects, the patient is responsive to IV diuretics. In other embodiments the patient is not responsive to IV diuretics.

In certain embodiments of the first to thirteenth aspects, the patient with HFpEF displays echocardiographic evidence of at least 2 of the following: left ventricular (LV) hypertrophy, left atrial (LA) enlargement, or diastolic dysfunction (medial mitral peak velocity/mitral annulus early diastolic recoil velocity [E/e′ ratio]≥15).

In certain embodiments of the first to thirteenth aspects, the patient with HFpEF displays hemodynamic evidence of elevated filling pressures, as indicated by pulmonary capillary wedge pressure ≥15 mmHg at rest or >25 mmHg with exercise, or left ventricular end-diastolic pressure ≥15 mmHg.

In certain embodiments of the first to thirteenth aspects, the patient with HFpEF is being treated with one or more cardiovascular medications at the same time the patient is being treated with an sGC stimulator. In certain embodiments, the one or more cardiovascular medications are selected from angiotensin-converting enzyme (ACE) inhibitors, angiotensin receptor blockers (ARBs), beta-blockers, MRAs, ARNIs, SGLT2 inhibitors (SGLT2 is), neprilysin inhibitors or medications comprising combinations of these thereof.

Clinical measurements of exercise and functional capacity in HFpEF patients include but are not limited to peak VO₂ (as measured by, e.g., CPET) and 6-minute walking distance (as measured by the 6-minute walk test [6MWT]). Clinical measurements of functional capacity also include NYHA classification class as well as measurements obtained using wearable or implanted monitors.

In some embodiments of the first to thirteenth aspects, the patient with HFpEF has limited exercise capacity. In some embodiments, the patient with limited exercise capacity displays a peak VO₂ value ≥60% of the age- and sex-adjusted normal value as measured by CPET. In other embodiments, the patient with limited exercise capacity displays a peak VO₂ value <60% of the age- and sex-adjusted normal value as measured by CPET. In some embodiments, the patient with limited exercise capacity displays a peak VO₂ value ≥70% of the age- and sex-adjusted normal value as measured by CPET. In other embodiments, the patient with limited exercise capacity displays a peak VO₂ value <70% of the age- and sex-adjusted normal value as measured by CPET. In some embodiments, the patient with limited exercise capacity displays a peak VO₂ value ≥80% of the age- and sex-adjusted normal value as measured by CPET. In other embodiments, the patient with limited exercise capacity displays a peak VO₂ value <80% of the age- and sex-adjusted normal value as measured by CPET.

In some embodiments of the first to thirteenth aspects, the patient with HFpEF has permanent or persistent atrial fibrillation. In other embodiments, the patient does not have permanent or persistent atrial fibrillation.

Relevant health status and quality of life (QOL) measurements in patients with HFpEF include but are not limited to those obtained by a number of self-reported questionnaires as summarized in the Examples section.

Relevant biomarkers associated with physiologic function and health status in patients with HFpEF include but are not limited to markers of cardiovascular health, markers of disease state, markers related to the mechanism of action of the drug, inflammatory markers, and fibrotic markers.

Relevant heart function measurements in patients with HFpEF include but are not limited to those obtained by the techniques of electrocardiogram (ECG) and echocardiography. Other relevant heart function measurements include cardiac magnetic resonance imaging (MRI), cardiac imaging with radioisotopes such as technetium, and exercise stress testing.

In some embodiments of the first to thirteenth aspects, the patient with HFpEF displays a metabolic/inflammatory phenotype.

In some embodiments of the first to thirteenth aspects, the patient with HFpEF has a history of hypertension. In some of these embodiments, the patient is being treated with at least 1 antihypertensive medication. In other embodiments, the patient has seated blood pressure (BP)>140/90 mmHg. In other embodiments, the patient is being treated with a stable regimen of one or more antihypertensive medications.

In certain embodiments of the first to thirteenth aspects, the patient with HFpEF has systolic BP≥130 mmHg and/or diastolic BP≥85 mmHg.

In certain embodiments of the first to thirteenth aspects, the patient with HFpEF has a fasting blood glucose level of 120 mg/dL or higher. In other embodiments, the patient with HFpEF has a fasting blood glucose level of 110 mg/dL or higher. In other embodiments, the patient with HFpEF has a fasting blood glucose level of 100 mg/dL or higher. In other embodiments, the patient with HFpEF has a fasting blood glucose level of 95 mg/dL or higher. In still other embodiments, the patient with HFpEF has been diagnosed as having type 2 diabetes mellitus or prediabetes. In some of these embodiments, the patient is being treated for diabetes or prediabetes. In yet other embodiments, the patient has a value of hemoglobin A1c≥5.6 as measured in the 6 months, 5 months, 4 months or 3 months before the start of treatment.

In certain embodiments of the first to thirteenth aspects, the patient with HFpEF has a waist circumference of 88 cm (35 inches) or more. In certain embodiments of the first to thirteenth aspects, the patient with HFpEF has a waist to hip ratio (WHR)≤0.8. In other embodiments 0.81≤WHR≤0.85. In still other embodiments, WHR≥0.86.

In some embodiments of the first to thirteenth aspects, the patient with HFpEF has a body mass index (BMI)>30 kg/m². In some embodiments, the patient with HFpEF has a BMI>25 kg/m². In other embodiments, the patient with HFpEF has a BMI>35 kg/m². In still other embodiments, the patient with HFpEF has a BMI>40 kg/m².

In some embodiments of the first to thirteenth aspects, the patient with HFpEF is of normal weight (BMI between 18.5 and 25). In other embodiments, she is overweight (BMI between 25 and 30). In other embodiments, she is obese (BMI equal or higher than 30). In still other embodiments, the patient is severely obese (BMI equal or higher than 40).

In some embodiments of the first to thirteenth aspects, the patient has kidney disease. In some of these embodiments, the patient has chronic kidney disease (CKD). In some embodiments, the patient has diabetic CKD. In some embodiments, the patient has diabetic nephropathy.

In some embodiments of the first to thirteenth aspects, the patient has fatty liver disease. In some embodiments, the patient has non-alcoholic fatty liver disease (NAFLD). In other embodiments, the patient has non-alcoholic steatohepatitis (NASH). In some embodiments, the patient has been diagnosed as having NAFLD or NASH. In other embodiments, the patient shows certain biomarker levels suggestive of fatty liver disease.

In some embodiments of the first to thirteenth aspects, the patient has pulmonary hypertension. In some embodiments, the patient has pulmonary arterial hypertension (PAH). In some embodiments, the patient has been diagnosed as having pulmonary hypertension or as having PAH. In other embodiments, the patient shows certain biomarker levels that suggest the patient has pulmonary hypertension or has PAH.

In certain embodiments of the first to thirteenth aspects, the patient with HFpEF has a HOMA-IR level of 1.9 or higher, indicative of early insulin resistance. In other embodiments, the patient with HFpEF has a HOMA-IR level of 2.9 or higher, indicative of significant insulin resistance.

The New York Heart Association (NYHA) classification provides a simple way of classifying the extent of HF. It classifies patients according to one of four categories based on their limitations during ordinary physical activity; the limitations/symptoms are in regards to normal breathing and varying degrees in shortness of breath, and/or angina pain. The four classes contemplated are:
Class I: No symptoms and no limitation in ordinary physical activity, e.g., shortness of breath when walking, climbing stairs, etc. Ordinary physical activity does not cause undue fatigue, palpitation, or dyspnea (shortness of breath).
Class II: Mild symptoms (mild shortness of breath and/or angina) and slight limitation during ordinary activity. Comfortable at rest. Ordinary physical activity results in fatigue, palpitation, dyspnea (shortness of breath).
Class III: Marked limitation in activity due to symptoms, even during less-than-ordinary activity, e.g., walking short distances (20-100 m). Comfortable only at rest. Less than ordinary activity results in fatigue, palpitation, dyspnea, or anginal pain.
Class IV: Severe limitations. Experiences symptoms even while at rest. If any physical activity is undertaken, discomfort is increased. Mostly bedbound patients.

In some embodiments of the first to thirteenth aspects, the patient with HFpEF display symptomatology of NYHA Class II, Class III, or Class IV, as assessed by a medical professional. In some embodiments, the patient displays symptomatology of NYHA Class II. In some embodiments, the patient displays symptomatology of NYHA Class III. In some embodiments, the patient displays symptomatology of NYHA Class IV.

In some embodiments of the first to thirteenth aspects, the patient is of white race, black race, or is a Native American or Asian American. In some embodiments, the patient is black. In other embodiments, the patient is an African American. In other embodiments, the patient is a Native American. In still other embodiments, the patient is an Asian American. In yet further embodiments, the patient is Asian. In yet other embodiments, the patient is African. In yet further embodiments, the patient is white. In yet other embodiments, the patient is of mixed race or mixed ethnicity.

In certain embodiments of the first to thirteenth aspects of the present invention, the patient is administered a single oral daily dose of Compound I-1 of between 10 mg and 40 mg, between 10 mg and 20 mg, between 20 mg and 40 mg, between 20 mg and 30 mg, or between 30 mg and 40 mg.

In certain embodiments of the first to thirteenth aspects described herein the patient is administered a single oral daily dose of 10 mg of Compound I-1.

In certain embodiments of the first to thirteenth aspects described herein the patient is administered a single oral daily dose of 20 mg of Compound I-1.

In certain embodiments of the first to thirteenth aspects described herein the patient is administered a single oral daily dose of 30 mg of Compound I-1.

In certain embodiments of the first to thirteenth aspects described herein the patient is administered a single oral daily dose of 40 mg of Compound I-1.

In certain embodiments, the first to thirteenth aspects described herein the patient is administered an oral dose of 5 mg of Compound I-1 twice per day (BID). In one embodiment, the patient is administered a first oral dose of 5 mg and a second oral dose of 5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 10 mg of Compound I-1 twice per day (BID). In one embodiment, the patient is administered a first oral dose of 10 mg and a second oral dose of 10 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 20 mg of Compound I-1 twice per day (BID). In one embodiment, the patient is administered a first oral dose of 20 mg and a second oral dose of 20 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 15 mg of Compound I-1 twice per day (BID). In one embodiment, the patient is administered a first oral dose of 15 mg and a second oral dose of 15 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is continuously administrate red a dose between 10 mg and 40 mg of Compound I-1 once per day indefinitely as long as the patient continues to experience clinical benefit. Accordingly, in some embodiments, the first to thirteenth aspects described herein comprise continuously administering to the patient an oral dose of between 10 mg and 40 mg once per day (QD), wherein the administration continues indefinitely as long as the patient continues to experience clinical benefit.

In other embodiments, the first to thirteenth aspects described herein comprise the continuous administration of a dose between 5 mg and 20 mg of Compound I-1 twice per day (BID) to the patient and continues indefinitely as long as the patient continues to experience clinical benefit. Accordingly, in some embodiments, the first to thirteenth aspects described herein comprise administering to the patient an oral dose of between 5 mg and 20 mg twice per day (BID), wherein the administration continues indefinitely as long as the patient continues to experience clinical benefit.

In some embodiments, the first to thirteenth aspects described herein comprise the administration of an initial dose of between 5 mg and 20 mg of Compound I-1 once per day (QD) to the patient for a period between 7 and 14 days, followed by an increase to a maintenance dose of between 10 mg and 40 mg (QD). In some embodiments, the maintenance dose continues indefinitely as long as the patient continues to experience clinical benefit. Accordingly, in some embodiments, the first to thirteenth aspects described herein comprise administering to the patient an initial oral dose of between 5 mg and 20 mg once per day (QD) for a period of between 7 days and 14 days, and subsequently administering to the patient a maintenance dose of between 10 mg and 40 mg QD. In some embodiments, the administration of the maintenance dose continues indefinitely as long as the patient continues to experience clinical benefit.

In other embodiments, the first to thirteenth aspects described herein comprise the administration of an initial dose of between 10 mg and 40 mg of Compound I-1 once per day (QD) to the patient, followed by a decrease to a maintenance dose of between 5 mg and 20 mg QD if the patient experiences hypotension. In some embodiments, the maintenance dose continues indefinitely as long as the patient continues to experience clinical benefit and the undesired hypotensive effects are minimized. Accordingly, in some embodiments, the first to thirteenth aspects described herein comprise administering to the patient an initial oral dose of between 10 mg and 40 mg QD, and subsequently administering to the patient a maintenance dose of between 5 mg and 20 mg QD if the patient experiences hypotension. In some embodiments, the administration of the maintenance dose continues indefinitely as long as the patient continues to experience clinical benefit and undesired hypotensive effects are minimized.

In certain embodiments of the first to thirteenth aspects of the present invention, the patient is administered a single oral daily dose of Compound I-2 of between 5 mg and 25 mg, between 5 mg and 20 mg, between 10 mg and 20 mg, between 15 mg and 25 mg, between 15 mg and 20 mg, or between 10 mg and 30 mg.

In certain embodiments of the first to thirteenth aspects described herein the patient is administered a single oral daily dose of between 5 mg and 10 mg of Compound I-2.

In certain embodiments of the first to thirteenth aspects described herein the patient is administered a single oral daily dose of between 5 mg and 15 mg of Compound I-2.

In certain embodiments of the first to thirteenth aspects described herein the patient is administered a single oral daily dose of between 10 mg and 20 mg of Compound I-2.

In certain embodiments of the first to thirteenth aspects described herein the patient is administered a single oral daily dose of between 15 mg and 20 mg of Compound I-2.

In certain embodiments of the first to thirteenth aspects described herein the patient is administered a single oral daily dose of between 10 mg and 15 mg of Compound I-2.

In certain embodiments of the first to thirteenth aspects described herein the patient is administered a single oral daily dose of between 15 mg and 25 mg of Compound I-2.

In certain embodiments of the first to thirteenth aspects described herein the patient is administered a single oral daily dose of between 20 mg and 25 mg of Compound I-2.

In certain embodiments of the first to thirteenth aspects described herein the patient is administered a single oral daily dose of between 20 mg and 35 mg of Compound I-2.

In certain embodiments of the first to thirteenth aspects described herein the patient is administered a single oral daily dose of between 25 mg and 35 mg of Compound I-2.

In certain embodiments of the first to thirteenth aspects described herein the patient is administered a single oral daily dose of 6 mg, 9 mg, 12 mg, 15 mg, 18 mg, 21 mg, 24 mg, 27 mg, 30 mg or 33 mg. In certain embodiments of the first to thirteenth aspects described herein the patient is administered a single oral daily dose of 6 mg, 9 mg, 12 mg, 15 mg, 18 mg, 21 mg or 24 mg. In certain embodiments of the first to thirteenth aspects described herein the patient is administered a single oral daily dose of 6 mg, 9 mg, 12 mg, 15 mg, or 18 mg.

In certain embodiments, the first to thirteenth aspects described herein the patient is administered an oral dose of between 2.5 mg and 5 mg of Compound I-2 twice per day (BID). In one embodiment, the patient is administered a first oral dose of between 2.5 mg and 5 mg and a second oral dose of between 2.5 mg and 5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 2.5 mg and 7.5 mg of Compound I-2 twice per day (BID). In one embodiment, the patient is administered a first oral dose of between 2.5 mg and 7.5 mg and a second oral dose of between 2.5 mg and 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 5 mg and 10 mg of Compound I-2 twice per day (BID). In one embodiment, the patient is administered a first oral dose of between 5 mg and 10 mg and a second oral dose of between 5 mg and 10 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 7.5 mg and 10 mg of Compound I-2 twice per day (BID). In one embodiment, the patient is administered a first oral dose of between 7.5 mg and 10 mg and a second oral dose of between 7.5 mg and 10 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 5 mg and 7.5 mg of Compound I-2 twice per day (BID). In one embodiment, the patient is administered a first oral dose of between 5 mg and 7.5 mg and a second oral dose of between 5 mg and 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 7.5 mg and 12.5 mg of Compound I-2 twice per day (BID). In one embodiment, the patient is administered a first oral dose of between 7.5 mg and 12.5 mg and a second oral dose of between 7.5 mg and 12.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 10 mg and 12.5 mg of Compound I-2 twice per day (BID). In one embodiment, the patient is administered a first oral dose of between 10 mg and 12.5 mg and a second oral dose of between 10 mg and 12.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 10 mg and 17.5 mg of Compound I-2 twice per day (BID). In one embodiment, the patient is administered a first oral dose of between 10 mg and 17.5 mg and a second oral dose of between 10 mg and 17.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of between 12.5 mg and 17.5 mg of Compound I-2 twice per day (BID). In one embodiment, the patient is administered a first oral dose of between 12.5 mg and 17.5 mg and a second oral dose of between 12.5 mg and 17.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 3 mg of Compound I-2 twice per day (BID). In one embodiment, the patient is administered a first oral dose of 3 mg and a second oral dose of 3 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 4.5 mg of Compound I-2 twice per day (BID). In one embodiment, the patient is administered a first oral dose of 4.5 mg and a second oral dose of 4.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 6 mg of Compound I-2 twice per day (BID). In one embodiment, the patient is administered a first oral dose of 6 mg and a second oral dose of 6 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 7.5 mg of Compound I-2 twice per day (BID). In one embodiment, the patient is administered a first oral dose of 7.5 mg and a second oral dose of 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 9 mg of Compound I-2 twice per day (BID). In one embodiment, the patient is administered a first oral dose of 9 mg and a second oral dose of 9 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 10.5 mg of Compound I-2 twice per day (BID). In one embodiment, the patient is administered a first oral dose of 10.5 mg and a second oral dose of 10.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 12 mg of Compound I-2 twice per day (BID). In one embodiment, the patient is administered a first oral dose of 12 mg and a second oral dose of 12 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is continuously administrate red a dose between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg of Compound I-2 once per day indefinitely as long as the patient continues to experience clinical benefit. Accordingly, in some embodiments, the first to thirteenth aspects described herein comprise continuously administering to the patient an oral dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg once per day (QD), wherein the administration continues indefinitely as long as the patient continues to experience clinical benefit.

In other embodiments, the first to thirteenth aspects described herein comprise the continuous administration of a dose between 2.5 mg and 5 mg, between 2.5 mg and 7.5 mg, between 2.5 mg and 12.5 mg, between 2.5 mg and 10 mg, between 5 mg and 7.5 mg, between 5 mg and 10 mg, between 7.5 mg and 12.5 mg, between 7.5 mg and 10 mg, between 10 mg and 12.5 mg, between 10 mg and 17.5 mg, between 12.5 mg and 17.5 mg or between 5 mg and 15 mg of Compound I-2 twice per day (BID) to the patient and continues indefinitely as long as the patient continues to experience clinical benefit. Accordingly, in some embodiments, the first to thirteenth aspects described herein comprise administering to the patient an oral dose of between 2.5 mg and 5 mg, between 2.5 mg and 7.5 mg, between 2.5 mg and 12.5 mg, between 2.5 mg and 10 mg, between 5 mg and 7.5 mg, between 5 mg and 10 mg, between 7.5 mg and 12.5 mg, between 7.5 mg and 10 mg, between 10 mg and 12.5 mg, between 10 mg and 17.5 mg, between 12.5 mg and 17.5 mg or between 5 mg and 15 mg twice per day (BID), wherein the administration continues indefinitely as long as the patient continues to experience clinical benefit.

In some embodiments, the first to thirteenth aspects described herein comprise the administration of an initial dose of between 2.5 mg and 5 mg, between 2.5 mg and 7.5 mg, between 2.5 mg and 12.5 mg, between 2.5 mg and 10 mg, between 5 mg and 7.5 mg, between 5 mg and 10 mg, between 7.5 mg and 12.5 mg, between 7.5 mg and 10 mg, between 10 mg and 12.5 mg, between 10 mg and 17.5 mg, between 12.5 mg and 17.5 mg or between 5 mg and 15 mg of Compound I-2 once per day (QD) to the patient for a period between 7 and 14 days, followed by an increase to a maintenance dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg (i.e., double the initial dose) (QD). In some embodiments, the maintenance dose continues indefinitely as long as the patient continues to experience clinical benefit. Accordingly, in some embodiments, the first to thirteenth aspects described herein comprise administering to the patient an initial oral dose of between 2.5 mg and 5 mg, between 2.5 mg and 7.5 mg, between 2.5 mg and 12.5 mg, between 2.5 mg and 10 mg, between 5 mg and 7.5 mg, between 5 mg and 10 mg, between 7.5 mg and 12.5 mg, between 7.5 mg and 10 mg, between 10 mg and 12.5 mg, between 10 mg and 17.5 mg, between 12.5 mg and 17.5 mg or between 5 mg and 15 mg once per day (QD) for a period of between 7 days and 14 days, and subsequently administering to the patient a maintenance dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg QD. In some embodiments, the administration of the maintenance dose continues indefinitely as long as the patient continues to experience clinical benefit.

In other embodiments, the first to thirteenth aspects described herein comprise the administration of an initial dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg of Compound I-2 once per day (QD) to the patient, followed by a decrease to a maintenance dose of between 2.5 mg and 5 mg, between 2.5 mg and 7.5 mg, between 2.5 mg and 12.5 mg, between 2.5 mg and 10 mg, between 5 mg and 7.5 mg, between 5 mg and 10 mg, between 7.5 mg and 12.5 mg, between 7.5 mg and 10 mg, between 10 mg and 12.5 mg, between 10 mg and 17.5 mg, between 12.5 mg and 17.5 mg or between 5 mg and 15 mg (i.e., half of the initial dose) QD if the patient experiences hypotension. In some embodiments, the maintenance dose continues indefinitely as long as the patient continues to experience clinical benefit and the undesired hypotensive effects are minimized. Accordingly, in some embodiments, the first to thirteenth aspects described herein comprise administering to the patient an initial oral dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg QD, and subsequently administering to the patient a maintenance dose of between 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg QD if the patient experiences hypotension. In some embodiments, the administration of the maintenance dose continues indefinitely as long as the patient continues to experience clinical benefit and undesired hypotensive effects are minimized.

In certain embodiments of the first to thirteenth aspects of the present invention, the patient is administered a single oral daily dose of Compound II-1 of between 2 mg and 20 mg, between 2.5 mg and 20 mg, between 5 mg and 20 mg, between 5 mg and 15 mg, or between 2.5 mg and 15 mg.

In certain embodiments of the first to thirteenth aspects described herein the patient is administered a single oral daily dose of 2.5 mg of Compound II-1.

In certain embodiments of the first to thirteenth aspects described herein the patient is administered a single oral daily dose of 5 mg of Compound II-1.

In certain embodiments of the first to thirteenth aspects described herein the patient is administered a single oral daily dose of 10 mg of Compound II-1.

In certain embodiments of the first to thirteenth aspects described herein the patient is administered a single oral daily dose of 15 mg of Compound II-1.

In certain embodiments, the first to thirteenth aspects described herein the patient is administered an oral dose of 1.25 mg of Compound II-1 twice per day (BID). In one embodiment, the patient is administered a first oral dose of 1.25 mg and a second oral dose of 1.25 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 2.5 mg of Compound II-1 twice per day (BID). In one embodiment, the patient is administered a first oral dose of 2.5 mg and a second oral dose of 2.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 5 mg of Compound II-1 twice per day (BID). In one embodiment, the patient is administered a first oral dose of 5 mg and a second oral dose of 5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is administered an oral dose of 7.5 mg of Compound II-1 twice per day (BID). In one embodiment, the patient is administered a first oral dose of 7.5 mg and a second oral dose of 7.5 mg, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In certain embodiments, the first to thirteenth aspects described herein the patient is continuously administrate red a dose between 2.5 mg and 20 mg, between 5 mg and 20 mg, between 5 mg and 15 mg, or between 2.5 mg and 15 mg of Compound II-1 once per day indefinitely as long as the patient continues to experience clinical benefit. Accordingly, in some embodiments, the first to thirteenth aspects described herein comprise continuously administering to the patient an oral dose of between 2.5 mg and 20 mg, between 5 mg and 20 mg, between 5 mg and 15 mg, or between 2.5 mg and 15 mg once per day (QD), wherein the administration continues indefinitely as long as the patient continues to experience clinical benefit.

In other embodiments, the first to thirteenth aspects described herein comprise the continuous administration of a dose between 1.25 mg and 10 mg, between 2.5 mg and 10 mg, between 2.5 mg and 7.5 mg, or between 1.25 mg and 7.5 mg of Compound II-1 twice per day (BID) to the patient and continues indefinitely as long as the patient continues to experience clinical benefit. Accordingly, in some embodiments, the first to thirteenth aspects described herein comprise administering to the patient an oral dose of between 1.25 mg and 10 mg, between 2.5 mg and 10 mg, between 2.5 mg and 7.5 mg, or between 1.25 mg and 7.5 mg twice per day (BID), wherein the administration continues indefinitely as long as the patient continues to experience clinical benefit.

In some embodiments, the first to thirteenth aspects described herein comprise the administration of an initial dose of between 1.25 mg and 10 mg, between 2.5 mg and 10 mg, between 2.5 mg and 7.5 mg, or between 1.25 mg and 7.5 mg of Compound II-1 once per day (QD) to the patient for a period between 7 and 14 days, followed by an increase to a maintenance dose of between 2.5 mg and 20 mg, between 5 mg and 20 mg, between 5 mg and 15 mg, or between 2.5 mg and 15 mg (i.e., double the initial dose) (QD). In some embodiments, the maintenance dose continues indefinitely as long as the patient continues to experience clinical benefit. Accordingly, in some embodiments, the first to thirteenth aspects described herein comprise administering to the patient an initial oral dose of between 1.25 mg and 10 mg, between 2.5 mg and 10 mg, between 2.5 mg and 7.5 mg, or between 1.25 mg and 7.5 mg once per day (QD) for a period of between 7 days and 14 days, and subsequently administering to the patient a maintenance dose of between 2.5 mg and 20 mg, between 5 mg and 20 mg, between 5 mg and 15 mg, or between 2.5 mg and 15 mg QD. In some embodiments, the administration of the maintenance dose continues indefinitely as long as the patient continues to experience clinical benefit.

In other embodiments, the first to thirteenth aspects described herein comprise the administration of an initial dose of between 2.5 mg and 20 mg, between 5 mg and 20 mg, between 5 mg and 15 mg, or between 2.5 mg and 15 mg of Compound II-1 once per day (QD) to the patient, followed by a decrease to a maintenance dose of between 1.25 mg and 10 mg, between 2.5 mg and 10 mg, between 2.5 mg and 7.5 mg, or between 1.25 mg and 7.5 mg (i.e. half of the initial dose) QD if the patient experiences hypotension. In some embodiments, the maintenance dose continues indefinitely as long as the patient continues to experience clinical benefit and the undesired hypotensive effects are minimized. Accordingly, in some embodiments, the first to thirteenth aspects described herein comprise administering to the patient an initial oral dose of between 2.5 mg and 20 mg, between 5 mg and 20 mg, between 5 mg and 15 mg, or between 2.5 mg and 15 mg QD, and subsequently administering to the patient a maintenance dose of between 1.25 mg and 10 mg, between 2.5 mg and 10 mg, between 2.5 mg and 7.5 mg, or between 1.25 mg and 7.5 mg QD if the patient experiences hypotension. In some embodiments, the administration of the maintenance dose continues indefinitely as long as the patient continues to experience clinical benefit and undesired hypotensive effects are minimized.

In some embodiments, the first to thirteenth aspects described herein result in an improvement in exercise capacity in the patient as measured by an increase in peak VO₂.

In some embodiments, the first to thirteenth aspects described herein result in an improvement in NYHA functional class in the patient as assessed by a medical professional. In some embodiments, the method delays or prevents clinical worsening of the patient as assessed by a medical professional using the NYHA functional class scale.

In some embodiments, the first to thirteenth aspects described herein result in a delay in clinical worsening in the patient or a reduction in the risk of hospitalization or the risk of re-hospitalization due to HF, or a reduction in the risk of cardiovascular death or all-cause mortality.

In certain embodiments, the patient can be treated with the sGC stimulator of the invention alone or in combination with an additional therapeutic agent for 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 6 months, 1 year, 2 years, or longer. In certain embodiments, the patient can be treated chronically. In some embodiments, the sGC stimulator of the invention can be used for the chronic treatment of HFpEF or any of its related symptoms. In some embodiments, the patient can be treated with an sGC stimulator of the invention alone or in combination with an additional therapeutic agent according to the methods described herein continuously, as long as the patient continues to experience clinical benefit.

### Combination Therapies

According to the first to thirteenth aspects, the invention also relates to the treatment of post-menopausal women with HFpEF with an sGC stimulator, pharmaceutically acceptable salts thereof, or pharmaceutical compositions and dosage forms comprising the sGC stimulator or the pharmaceutically acceptable salt thereof, in combination with other therapeutic agents.

In some particular embodiments of the first to thirteenth aspects, a compound of the invention, a pharmaceutically acceptable salt thereof, a pharmaceutical composition or a dosage form thereof, can be used for the treatment of HFpEF in combination with one or more medications that are independently selected from antihypertensive medications, blood glucose-reducing medications, anti-hyperlipidemics, renoprotective medications, and neprilysin inhibitors. In other embodiments of the first to thirteenth aspects, a compound of the invention, a pharmaceutically acceptable salt thereof, a pharmaceutical composition or a dosage form thereof, can be used for the treatment of HFpEF in combination with one or more medications selected from a platelet aggregation inhibitor.

As used herein, the terms “in combination” (as in the sentence “in combination therapy”) or “co-administration” can be used interchangeably to refer to the use of more than one therapy. The use of the terms does not restrict the order in which the therapies are administered to a patient.

For combination treatment with more than one therapeutic agent, wherein the therapeutic agents are in separate dosage formulations or dosage forms, the therapeutic agents may be administered separately or in conjunction (i.e., at the same time). In addition, when administered separately, the administration of one therapeutic agent may be prior to or subsequent to the administration of the other agent.

When the sGC stimulator is used in combination therapy with other therapeutic agents, a therapeutically effective amount of the other therapeutic agent or each of the other therapeutic agents will depend on the type of drug used. Suitable dosages are known for approved therapeutic agents and can be adjusted by the skilled artisan according to the condition of the patient, the type of condition(s) being treated, and the amount of a the sGC stimulator being used. In one embodiment of this invention, the sGC stimulator and the additional therapeutic agent(s) are each administered in a therapeutically effective amount (i.e., each in an amount that would be therapeutically effective if administered alone). In other embodiments, the sGC stimulator and the additional therapeutic agent(s) are each administered in an amount that alone would not provide a therapeutic effect (i.e., a sub-therapeutic dose). In yet another embodiment, the sGC stimulator can be administered in an effective therapeutic amount, while the additional therapeutic agent(s) are administered in a sub-therapeutic dose. In still another embodiment, the sGC stimulator can be administered in a sub-therapeutic dose, while the additional therapeutic agent(s) are administered in a therapeutically effective amount.

When co-administration involves the separate administration of a first amount of the sGC stimulator and a second amount of an additional therapeutic agent, the compounds are administered sufficiently close in time as to produce the desired therapeutic effect. For example, the period of time between each administration that can result in the desired therapeutic effect can range from minutes to hours and can be determined by taking into account the properties of each compound, such as potency, solubility, bioavailability, plasma half-life (T_{1/2}), and pharmacokinetic (PK) profile. For example, the sGC stimulator and a second therapeutic agent can be administered in any order within 24 hours of each other, within 16 hours of each other, within 8 hours of each other, within 4 hours of each other, within 1 hour of each other, within 30 minutes of each other, within 5 minutes of each other, simultaneously, or concomitantly.

More specifically, a first therapy can be administered to a patient prior to (e.g., 5 minutes, 15 minutes, 30 minutes, 45 minutes, 1 hour, 2 hours, 4 hours, 6 hours, or 12 hours before), concomitantly with, or subsequent to (e.g., 5 minutes, 15 minutes, 30 minutes, 45 minutes, 1 hour, 2 hours, 4 hours, 6 hours, 12 hours after) the administration of a second therapy.

Examples of other therapeutic agents that may be combined with an sGC stimulator according the first to thirteenth aspects of the invention include but are not limited to those discussed below.

1. Blood glucose-lowering medications (also referred as glycemic control medications or antidiabetic medications) that may be used in combination with The sGC stimulator of the invention include but are not limited to:

Biguanides. Generally, the biguanide metformin is the first medication that is prescribed to treat type 2 diabetes. It works by improving the sensitivity of body tissues to insulin so that the body uses insulin more effectively. Metformin also lowers glucose production in the liver. Metformin may not lower blood sugar enough on its own. If metformin and lifestyles changes are not enough to control blood sugar levels, other oral or injected medications can be added, such as the types noted below.

Sulfonylureas. Examples of medications in this class include glyburide, glybenclamide, glipizide, gliclazide, gliquidone, glimepiride, atorvastatin calcium combined with glimepiride, meglinatide, tolbutamide, chlorpropamide, acetohexamide, and tolazamide. In certain embodiments, the sulfonylurea that can be used in combination with The sGC stimulator of the invention in the treatment of HFpEF is selected from glyburide, glipizide, and glimepiride.

Alpha-glucosidase inhibitors. For example, acarbose, epalrestat, voglibose, and miglitol.

Insulin secretagoges. Examples include repaglinide, mitiglinide, and nateglinide. In certain embodiments, the insulin secretagoge that can be used in combination with The sGC stimulator of the invention in the treatment of HFpEF is repaglinide or nateglinide.

Thiazolidinediones. For example, rosiglitazone, troglitazone, ciglitazone, pioglitazone, englitazone, lobeglitazone sulfate, and balaglitazone.

DPP-4 (Dipeptidyl peptidase-4) inhibitors (or DPP-IV inhibitors). Examples of these medications include sitagliptin, vildagliptin, saxagliptin, alogliptin, linagliptin, alogliptin benzoate combined with metformin or metformin hydrochloride, anagliptin, teneligliptin, atorvastatin calcium and glimepiride, empagliflozin combined with linagliptin, gemigliptin, sitagliptin phosphate monohydrate combined with pioglitazone hydrochloride, sitagliptin combined with pioglitazone, sitagliptin combined with atorvastatin calcium, and (2S,4S)-1-[2-(1,1-dimethyl-3-oxo-3-pyrrolidin-1-yl-propylamino) acetyl]-4-fluoro-pyrrolidine-2-carbonitrile (DBPR-108). In certain embodiments, the DDP-4 inhibitor that can be used in combination with The sGC stimulator of the invention in the treatment of HFpEF is sitagliptin, saxagliptin, or linagliptin.

GLP-1 (Glucagon-like peptide-1) receptor agonists or incretin mimetics. Examples include exenatide, dulaglutide, liraglutide, semaglutide, lixisenatide, lixisenatide combined with insulin glargine, albiglutide and pegapamodutide (TT-401), tirzepatide (LY3298176) (dual glucose-dependent insulinotropic polypeptide [GIP] and GLP-1 receptor agonist). In certain embodiments, the GLP-1 receptor agonist that can be used in combination with The sGC stimulator of the invention in the treatment of HFpEF is exenatide, semaglutide, or liraglutide.

SGLT2 inhibitors (SGLT2 is). Examples include empagliflozin, empaglifozin combined with linagliptin, empagliflozin combined with metformin, ipragliflozin, ipragliflozin L-proline, tofogliflozin, sergliflozin etabonate, remogliflozin ctabonate, ertugliflozin, ertugliflozin combined with sitagliptin, ertugliflozin combined with metformin, sotagliflozin, canagliflozin, canagliflozin combined with metformin or metformin hydrochloride, dapagliflozin, dapagliflozin combined with metformin, or metformin hydrochloride combined with luscoglifozin, or dapagliflozin combined with saxagliptin. In one embodiment, the SGLT2 inhibitor is empagliflozin, ertuglifozin, canagliflozin, or dapagliflozin, or is a combination of drugs containing these agents. In another embodiment, the SGLT2 inhibitor is dapagliflozin. In another embodiment, the SGLT2 inhibitor is empagliflozin. In another embodiment, the SGLT2 inhibitor is canagliflozin. In certain embodiments, SGLT2 inhibitor is canagliflozin or dapagliflozin.

SGLT1 inhibitors or combinations of SGLT1 and SGLT2 inhibitors. Example includes sotagliflozin.

Insulin therapy. There are many types of insulin therapies, and they each work in a different way. Options include insulin glulisine, insulin degludec, insulin lispro, insulin aspart, insulin glargine, insulin detemir, insulin isophane, insulin mixtard (human insulin containing both fast-acting [soluble] and long-acting [isophane] insulin), insulin degludec combined with insulin aspart, insulin human (rDNA origin) inhalation powder, recombinant human insulin, hepatic-directed vesicle insulin, insulin tregopi (IN-105), insulin degludec combined with liraglutide, insulin peglispro (LY-2605541), and nodlin.

Tolimidone (a lyn protein-tyrosine kinase activator).

2. Blood pressure (BP)-lowering medications (also known as anti-hypertensive medications) that may be used in combination with The sGC stimulator of the invention include, but are not limited to:

Diuretics. Diuretics, sometimes called water pills, are medications that act on the kidneys to help the body eliminate sodium and water, and in turn reduces blood volume. Diuretics or calcium channel blockers may work better for black and older people than the usc of angiotensin-converting enzyme (ACE) inhibitors alone. Thiazide diuretics are often the first, but not the only, choice for high blood pressure medications. Diuretics include, for example, chlorothiazide, chlorthalidone, hydrochlorothiazide, bendroflumethiazide, cyclopenthiazide, methyclothiazide, polythiazide, quinethazone, xipamide, metolazone, indapamide, cicletanine, furosemide, toresamide, amiloride, spironolactone, canrenoate potassium, eplerenone, triamterene, acetazolamide, and carperitide. In certain embodiments, the diuretic that can be used in combination with The sGC stimulator of the invention in the treatment of HFpEF is spironolactone.

Beta blockers. These medications reduce the workload on the heart and open the blood vessels, causing the heart to beat slower and with less force. When prescribed alone, beta blockers are less effective, especially in black and older people, but they may be more effective when combined with other BP-lowering medications. Beta blockers include, for example, acebutolol, atenolol, metoprolol, and nebivolol. In certain embodiments, the beta blocker that can be used in combination with The sGC stimulator of the invention in the treatment of HFpEF is metoprolol.

Angiotensin-converting enzyme (ACE) inhibitors. These medications help relax blood vessels by blocking the formation of a natural chemical that narrows blood vessels. ACE inhibitors that may be combined with The sGC stimulator of the invention in the treatment of HFpEF include, for example, sulfhydryl-containing agents (e.g, captopril, zofenopril), dicarboxylate-containing agents (e.g., enalapril, quinapril, ramipril, perindopril, lisinopropil, and benazepril), phosphonate-containing agents (e.g., fosinopril), naturally occurring ACE inhibitors (e.g., casokinins, lactokinins, lactotripeptides Val-Pro-Pro, and Ile-Pro-Pro), alacepril, delapril, cilazapril, imidapril, temocapril, moexipril, lisinopril, combinations of lisinopril with hydrochlorothiazide, trandolapril, and spirapril. In certain embodiments, the ACE inhibitor that can be used in combination with The sGC stimulator of the invention in the treatment of HFpEF is selected from lisinopril, combinations of lisinopril with hydrochlorothiazide, benazepril, captopril, and enalapril.

Angiotensin II receptor blockers (ARBs). These medications help relax blood vessels by blocking the action, not the formation, of a natural chemical that narrows blood vessels. ARBs include candesartan, losartan, losartan potassium-hydrochlorothiazide, valsartan, candesartan cilexetil, eprosaran, irbesartan, telmisartan, olmesartan medoxomil (or olmesartan), azilsartan medoxomil, azilsartan, amlodipine besylate combined with irbesartan, azilsartan combined with amlodipine besilate, cilnidipine combined with valsartan, fimasartan, irbesartan combined with atorvastatin, irbesartan combined with trichlormethiazide, losartan potassium combined with hydrochlorothiazide and/or amlodipine besylate, pratosartan, atorvastatin calcium combined with losartan potassium, nifedipine and candesartan cilexetil, sacubitril combined with valsartan or LCZ-696, angiotensin AT2 antagonist and TAK-591, and olmesartan medoxomil. In certain embodiments, the ARB that can be used in combination with The sGC stimulator of the invention in the treatment of HFpEF candesartan, losartan, eprosaran, irbesartan, olmesartan, telmisartan, and valsartan.

Endothelin Receptor antagonists (ERAs). For example, atrasentan, bosentan, sitaxentan, ambrisentan, actelion-1 (macitentan), Cyclo(D-trp-D-asp-L-pro-D-val-L-leu) (BQ-123), sparsentan, and tezosentan disodium. In some embodiments, the ERA is bosentan.

Mineralocorticoid receptor antagonists (MRAs). For example, spironolactone, amiloride hydrochloride combined with spironolactone, apararenone or MT-3995, eplerenone, and finerenone (BAY-94-8862). In some embodiments, the MRA is finerenone.

Calcium channel blockers. These medications help relax the muscles of the blood vessels. Calcium channel blockers may work better for black and older people than the use of ACE inhibitors alone. Some slow heart rate. Calcium channel blockers that can be combined with The sGC stimulator of the invention for the treatment of HFpEF include, for example, amlodipine, aranidipine, azelnidipine, barnidipine, benidipine, cilnidipine, clevidipine, diltiazem, efonidipine, felodipine, lacidipine, lercanidipine, manidipine, nicardipine, nifedipine, nilvadipine, nimodipine, nisoldipine, nitrendipine, pranidipine, isradipine, verapamil, gallopamil, diltiazem, mibefradil, bepridil, fluspirilene, and fendiline.

Renin inhibitors. Aliskiren slows down the production of renin, which is an enzyme produced by the kidneys that starts a chain of chemical steps that increases blood pressure. Aliskiren works by reducing the ability of renin to begin this process. Due to a risk of serious complications, including stroke, aliskiren cannot be taken without an ACE inhibitor or an ARB. However, aliskerin is contraindicated in patients with diabetes.

Alpha blockers. These medications reduce nerve impulses to blood vessels, in turn reducing the effects of natural chemicals that narrow blood vessels. Alpha blockers include doxazosin, prazosin, and others.

Alpha-beta blockers. In addition to reducing nerve impulses to blood vessels, alpha-beta blockers slow the heartbeat to reduce the amount of blood that must be pumped through the vessels. Alpha-beta blockers include carvedilol and labetalol.

Central-acting agents. These medications prevent the brain from signaling the nervous system to increase the heart rate and narrow blood vessels. Examples include clonidine, guanfacine, and methyldopa.

Vasodilators. These medications work directly on the muscles in the walls of the arteries, preventing the muscles from tightening and the arteries from narrowing. Examples of vasodilators include NO donors such nitroglycerine, hydralazine, and minoxidil.

Aldosterone antagonists. These drugs block the effect of a natural chemical that can lead to salt and fluid retention, which can contribute to high blood pressure. Examples are finerenone, spironolactone, and eplerenone

3. Anti-hyperlipidemic medications that may be used in combination with The sGC stimulator of the invention include, but are not limited to:

Statins. Examples of statins include, but are not limited to, atorvastatin fluvastatin, lovastatin, pitavastatin, pravastatin, rosuvastatin, and simvastatin. Combinations of statins with another agent can be also be used. Examples include, but are not limited to, amlodipine/atorvastatin, aspirin/pravastatin, ezetimibe/simvastatin, niacin/simvastatin, lovastatin/niacin, simvastatin/sitagliptin, and atorvastatin/ezetimibe. In certain embodiments, the statin is atorvastatin, lovastatin, pravastatin, rosuvastatin, or simvastatin.

Fibrates or fibric acid derivatives. Examples include, but are not limited to, fenofibrate, gemfibrozil, bezafibrate, ciprofibrate, clinofibrate, and clofibrate.

Niacin (or nicotinic acid).

Bile acid sequestrants. Examples include, but are not limited to, cholestyramine, colesevelam, colestilan, and colestipol.

Ezetimibe, lomitapide, phytosterols or orlistat.

PCSK9 inhibitors. Examples include, but are not limited to, alirocumab and evolocumab.

4. Neprilysin inhibitors (also known as endopeptidase inhibitors or NEP inhibitors or enkephalinase inhibitors). For example, sacubitril, or the combination of sacubitril with valsartan. Other neprilysin inhibitors in development that could be combined with The sGC stimulators of the invention include TD-1439 and TD-0714. In some embodiments, the neprilysin inhibitor is sacubitril, or combinations of sacubitril with other agents.

5. Renoprotective drugs. Examples include, but are not limited to, bardoxolone, ACE inhibitors (such as captopril), ARBs (such as losartan or irbesartan), SGLT2 inhibitors (such as canagliflozin), GLP1 receptor agonists, MRAs (such as finerenone), and ERAs (such as atrasentan).

6. Certain drugs used for the treatment of PAH. For example, iloprost, ambrisentan, epoprosterol, alprostadil or trepostinil.

7. Platelet aggregation inhibitors (or antiplatelet drug). Examples include, but are not limited to heparin, acetylsalicylic acid and clopidogrel.

In certain embodiments of the first to thirteenth aspects of the invention described herein, in addition to the sGC stimulator, the patient is further administered one or more (two, three, four, five, etc.) anti-hypertensive medications. In one embodiment, the one or more anti-hypertensive medications are each independently selected from an angiotensin-converting enzyme (ACE) inhibitor, an angiotensin II receptor blocker (ARB), an MR antagonist (MRA), an endothelin receptor antagonist (ERA), a diuretic and an SGLT2i or a medication comprising a combination of these thereof. In one embodiment, at least one of the anti-hypertensive medications is a diuretic. In a specific embodiment, the diuretic is a thiazide diuretic described above. In another specific embodiment, the diuretic is hydrochlorothiazide or spironolactone. In one embodiment, at least one of the anti-hypertensive medications is an MRA described herein. In a specific embodiment, the MRA is finerenone. In one embodiment, at least one of the anti-hypertensive medications is ERA described herein. In a specific embodiment, the ERA is sparsentan.

In certain embodiments of the first to thirteenth aspects of the invention described herein, in addition to the sGC stimulator, the patient is administered one or more (two, three, four, five, etc.) anti-hypertensive medications. In one embodiment, the one or more anti-hypertensive medications are each independently selected from an angiotensin-converting enzyme (ACE) inhibitor, an angiotensin II receptor blocker (ARB), and a diuretic. In another embodiment, the one or more anti-hypertensive medications is independently selected from the group consisting of lisinopril, combinations of lisinopril with hydrochlorothiazide, benazepril, captopril, enalapril, candesartan, losartan, azilsartan, cprosartan, irbesartan, olmesartan, telmisartan, and valsartan. In another embodiment, the one or more anti-hypertensive medications are each independently selected from the group consisting of lisinopril, combination of lisinopril and hydrochlorothiazide, enalapril, losartan, metoprolol, eplerenone, chlorthalidone, and spironolactone. In another embodiment, the one or more anti-hypertensive medications are each independently selected from the group consisting of lisinopril, combination of lisinopril and hydrochlorothiazide, enalapril, and losartan. In one embodiment, at least one of the anti-hypertensive medications is an angiotensin-converting enzyme (ACE) inhibitor or an angiotensin II receptor blocker (ARB). In another embodiment, at least one of the anti-hypertensive medications is selected from the group consisting of lisinopril, combination of lisinopril and hydrochlorothiazide, enalapril, and losartan.

In certain embodiments of the first to thirteenth aspects of the invention described herein, in addition to the sGC stimulator, the patient is administered one or more (two, three, four, five, etc.) blood glucose-lowering medications (anti hyperglycemic or antidiabetes drugs). In one embodiment, the one or more blood glucose-lowering medications are independently selected from the group consisting of insulin, metformin, glyburide, glipizide, glimepiride, repaglinide, nateglinide, sitagliptin, saxagliptin, linagliptin, exenatide, liraglutide, semaglutide, ertugliflozin, empagliflozin, canagliflozin, and dapagliflozin. In certain embodiments, insulin is not given or administered to the patient treated with the methods described herein during the treatment with the sGC stimulator. In some embodiments, the patient is being treated with an oral antihyperglycemic agent in addition to the sGC stimulator.

In certain embodiments of the first to thirteenth aspects described herein, in addition to the sGC stimulator, the patient is administered an anti-hypertensive medication described herein and a blood glucose-lowering medication described herein. In one embodiment, the patient if administered one or more anti-hypertensive medications independently selected from the group consisting of isinopril, combination of lisinopril and hydrochlorothiazide, enalapril, losartan, metoprolol, and spironolactone, and one or more blood glucose-lowering medications independently selected from the group consisting of insulin, metformin, and glipizide. In one embodiment, at least one of the anti-hypertensive medications is an angiotensin-converting enzyme (ACE) inhibitor or an angiotensin II receptor blocker (ARB). In another embodiment, at least one of the anti-hypertensive medications is selected from the group consisting of lisinopril, combination of lisinopril and hydrochlorothiazide, enalapril, and losartan.

In certain embodiments of the first to thirteenth aspects, in addition to the sGC stimulator, the patient is administered one or more (two, three, four, five, etc.) anti-hyperlipidemic medications. In one embodiment, the one or more anti-hyperlipidemic medications is selected from a cholesterol-lowering medication. In one embodiment, the one or more anti-hyperlipidemic medications is independently selected from the group consisting of atorvastatin, pravastatin, simvastatin, rosuvastatin, lovastatin, and nicotinic acid. In another embodiment, the one or more cholesterol-lowering medications is selected from the group consisting of atorvastatin, pravastatin, rosuvastatin, lovastatin, and simvastatin.

In certain embodiments of the first to thirteenth aspects described herein, in addition to the sGC stimulator, the patient is administered one or more (two, three, four, five, etc.) neprilysin inhibitors. In one embodiment, the neprilysin inhibitor is sacubitril or the combination of sacubitril with valsartan.

In certain embodiments of the first to thirteenth aspects described, in addition to the sGC stimulator, the patient is administered one or more (two, three, four, five, etc.) renoprotective medications. In certain embodiments, the first to thirteenth aspects described herein further comprise administering to the patient bardoxolone.

In certain embodiments of the first to thirteenth aspects described herein, in addition to the sGC stimulator, the patient is administered one or more (two, three, four, five, etc.) renoprotective medications. In one embodiment, the renoprotective medication is selected from the group consisting of irbesartan, losartan, captopril, finerenone, canagliflozin, and atrasentan.

In certain embodiments of the first to thirteenth aspects described herein, in addition to the sGC stimulator, the patient is administered one or more (two, three, four, five, etc.) medications for the treatment of PAH. In one embodiment, the one or more medications for the treatment of PAH are independently selected from the group consisting of iloprost, ambrisentan, epoprosterol, alprostadil, and trepostinil.

### sGC Stimulators of the Invention

### Definitions and General Terminology Relating to Compounds

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

For purposes of this disclosure, the chemical elements are identified in accordance with the Periodic Table of the Elements, Chemical Abstracts Service (CAS) version, and the Handbook of Chemistry and Physics, 101^{st} Ed. 2020-21. Additionally, general principles of organic chemistry are described in Organic Chemistry, Thomas Sorrell, University Science Books, Sausalito: 1999, and March's Advanced Organic Chemistry, 7th Ed., Smith, M. B. and March, J., eds. John Wiley & Sons, New York: 2013, which are herein incorporated by reference in their entirety.

Unless otherwise stated, all tautomeric forms of the compounds of the present disclosure are also within the scope of the invention.

When one or more position(s) of a structure can be substituted with one or more than one substituent selected from a specified group or list, the substituent or substituents at each position may be “independently selected” to be equal or the same at each position and for each instance, unless otherwise specified. For example, if a phenyl is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ may be fluoro and one may be methyl, or both may be chloro, etc. Similarly, if a substitutable atom is bonded to more than one hydrogen (e.g., CH₃ or NH₂), the substituents may be “independently selected” to be equal or the same at each position and for each instance, unless otherwise specified. For example, if a methyl (e.g., CH₃) is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ may be fluoro and one may be methyl (e.g., CHF(CH₃), or both may be chloro (e.g., CHCl₂), etc.

The phrase “up to”, as used herein, refers to any integer number that is equal to or less than the number following the phrase. For example, “up to 3” means any one of 0, 1, 2, or 3. As described herein, a specified number range of atoms includes any integer therein. For example, a group having from 1 through 4 atoms could have 1, 2, 3, or 4 atoms. When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. A chemically feasible compound is a compound that can be prepared by a person skilled in the art based on the disclosures herein, supplemented if necessary by relevant knowledge of the art.

In one embodiment, the present disclosure may include replacement of hydrogen with deuterium (i.e., 2H), which may afford certain therapeutic advantages resulting from greater metabolic stability (e.g., increased in vivo half-life or reduced dosage requirements) and hence may be preferred in some circumstances. Deuterium-labeled compounds of the present invention can generally be prepared by following procedures analogous to those disclosed in the Schemes and/or in the Examples herein below, by substituting a deuterated reagent for a non-deuterated reagent.

The term “alkyl” as in, for example, “alkyl chain” or “alkyl group”, as used herein, refers to a saturated unbranched (e.g., linear) or branched monovalent hydrocarbon radical. A Cₓ alkyl is an alkyl chain containing x carbon atoms, wherein x is an integer different from 0. A “C_{x-y} alkyl”, wherein x and y are two different integers, both different from 0, is an alkyl chain containing between x and y number of carbon atoms, inclusive. For example, a C₁₋₆ alkyl is an alkyl as defined above containing any number of between 1 and 6 carbon atoms. Examples of alkyl groups include, but are not limited to, methyl (i.e., C₁ alkyl), ethyl (i.e., C₂ alkyl), n-propyl (a C₃ alkyl), isopropyl (a different C₃ alkyl), n-butyl, isobutyl, s-butyl, t-butyl, pentyl, hexyl, heptyl, octyl and the like. In certain embodiments, the alkyl group is a C₁₋₄ alkyl. In certain embodiments, the alkyl group is a C₁₋₃ alkyl. In still other embodiments, the alkyl group is methyl.

As used herein, the terms “halogen” or “halo” mean F, Cl, Br, or I. In certain embodiments, the halo is F or Cl. In still other embodiments, the halo is F.

The term “hydroxyl” or “hydroxy” refers to —OH.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### Compound Embodiments

In some embodiments of the first to thirteenth aspects of the invention, the sGC stimulator is a compound of Formula I:

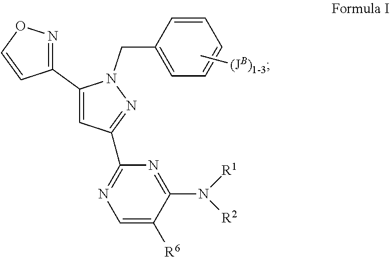

wherein:
each J^{B} is independently selected from halogen or C₁₋₃ alkyl;
R¹ is hydrogen or C₁₋₃ alkyl;
R² is a C₁₋₆ alkyl group optionally and independently substituted by up to three instances of R⁵,
R⁶ is selected from hydrogen or fluoro; and wherein
each R⁵ is independently selected from hydroxy, C₁₋₃ haloalkyl, halogen or —C(O)NH₂.

In some embodiments of Formula I, each J^{B} is independently selected from a halogen. In other embodiments, each J^{B} is fluoro. In other embodiments at least one J^{B} is halogen. In still other embodiments, at least one J^{B} is fluoro. In still other embodiments, two J^{B} are halogen, and the third is independently selected from halogen or methyl.

In some embodiments of Formula I, R¹ is hydrogen. In other embodiments, R¹ is methyl or ethyl. In other embodiments, R¹ is methyl.

In some embodiments of Formula I, R² is C₁₋₃ alkyl independently substituted by up to three instances of R⁵. In other embodiments, R² is a C₁₋₂ alkyl independently substituted by up to three instances of R⁵.

In some embodiments of Formula I, each R⁵ is independently selected from hydroxy, trifluoromethyl or —C(O)NH₂.

In some embodiments of Formula I, R⁶ is hydrogen. In other embodiments, R⁶ is fluoro.

In some embodiments of Formula I, the compound is selected from one depicted below, wherein Compound I-1 is praliciguat and Compound I-2 is olinciguat:

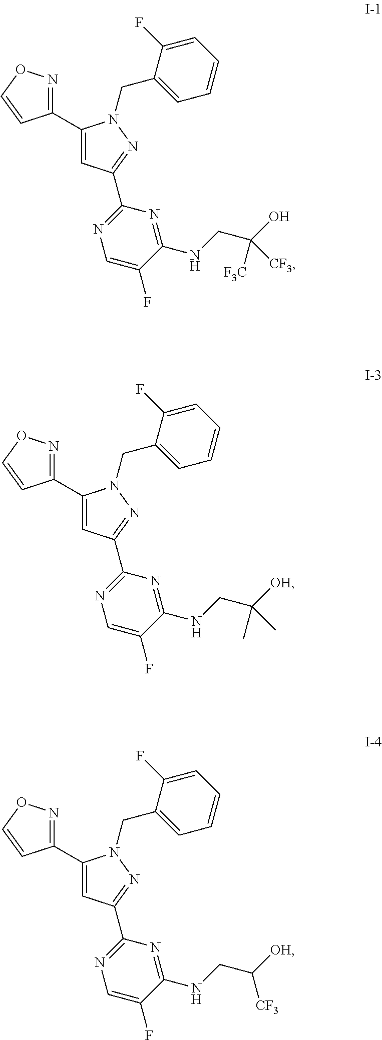 [SMILES: OC(CNc1nc(-c2cc(-c3ccon3)n(Cc3ccccc3F)n2)ncc1F)(C(F)(F)F)C(F)(F)F ; CC(C)(O)CNc1nc(-c2cc(-c3ccon3)n(Cc3ccccc3F)n2)ncc1F ; OC(CNc1nc(-c2cc(-c3ccon3)n(Cc3ccccc3F)n2)ncc1F)C(F)(F)F]

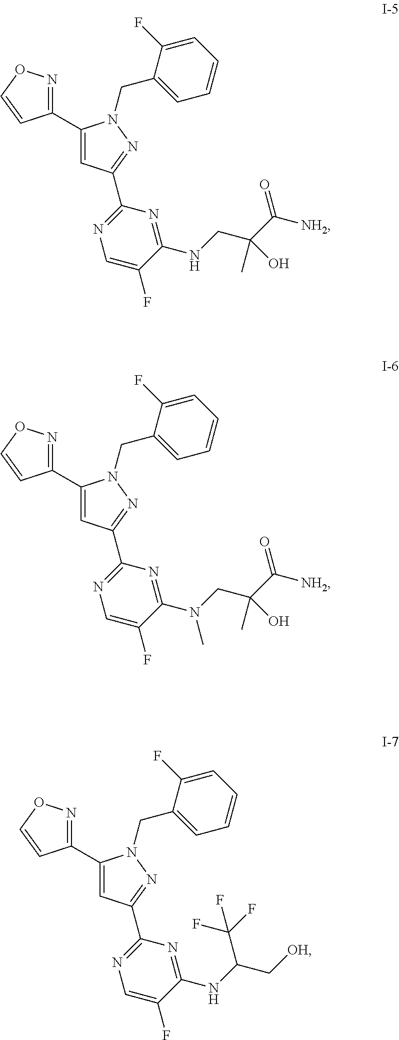 [SMILES: CC(O)(CNc1nc(-c2cc(-c3ccon3)n(Cc3ccccc3F)n2)ncc1F)C(N)=O ; CN(CC(C)(O)C(N)=O)c1nc(-c2cc(-c3ccon3)n(Cc3ccccc3F)n2)ncc1F ; OCC(Nc1nc(-c2cc(-c3ccon3)n(Cc3ccccc3F)n2)ncc1F)C(F)(F)F]

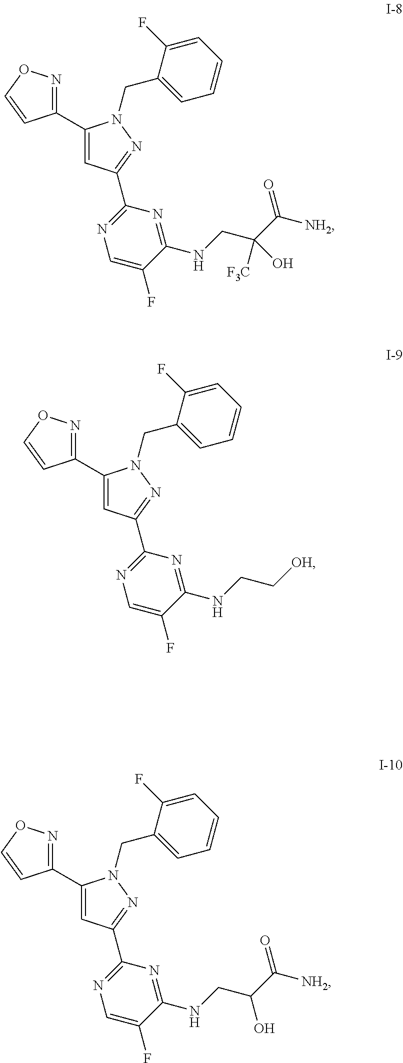 [SMILES: NC(=O)C(O)(CNc1nc(-c2cc(-c3ccon3)n(Cc3ccccc3F)n2)ncc1F)C(F)(F)F ; OCCNc1nc(-c2cc(-c3ccon3)n(Cc3ccccc3F)n2)ncc1F ; NC(=O)C(O)CNc1nc(-c2cc(-c3ccon3)n(Cc3ccccc3F)n2)ncc1F]

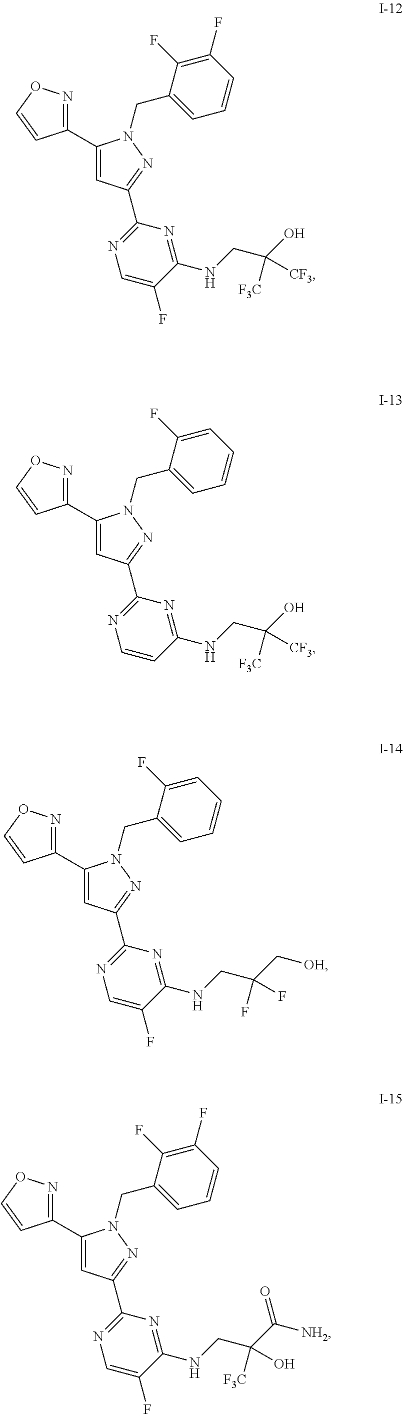 [SMILES: OC(CNc1nc(-c2cc(-c3ccon3)n(Cc3cccc(F)c3F)n2)ncc1F)(C(F)(F)F)C(F)(F)F ; OC(CNc1ccnc(-c2cc(-c3ccon3)n(Cc3ccccc3F)n2)n1)(C(F)(F)F)C(F)(F)F ; OCC(F)(F)CNc1nc(-c2cc(-c3ccon3)n(Cc3ccccc3F)n2)ncc1F ; NC(=O)C(O)(CNc1nc(-c2cc(-c3ccon3)n(Cc3cccc(F)c3F)n2)ncc1F)C(F)(F)F]

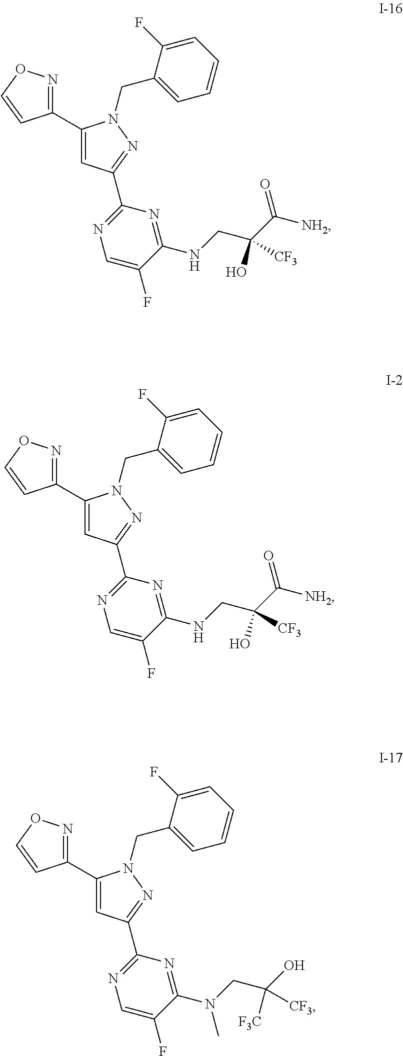 [SMILES: NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F ; CN(CC(O)(C(F)(F)F)C(F)(F)F)c1nc(-c2cc(-c3ccon3)n(Cc3ccccc3F)n2)ncc1F]

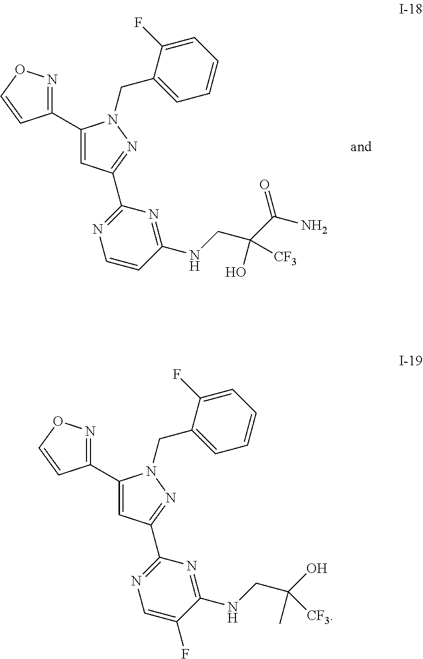 [SMILES: NC(=O)C(O)(CNc1ccnc(-c2cc(-c3ccon3)n(Cc3ccccc3F)n2)n1)C(F)(F)F ; CC(O)(CNc1nc(-c2cc(-c3ccon3)n(Cc3ccccc3F)n2)ncc1F)C(F)(F)F]

In some embodiments of Formula I, the compound is selected from one depicted below:

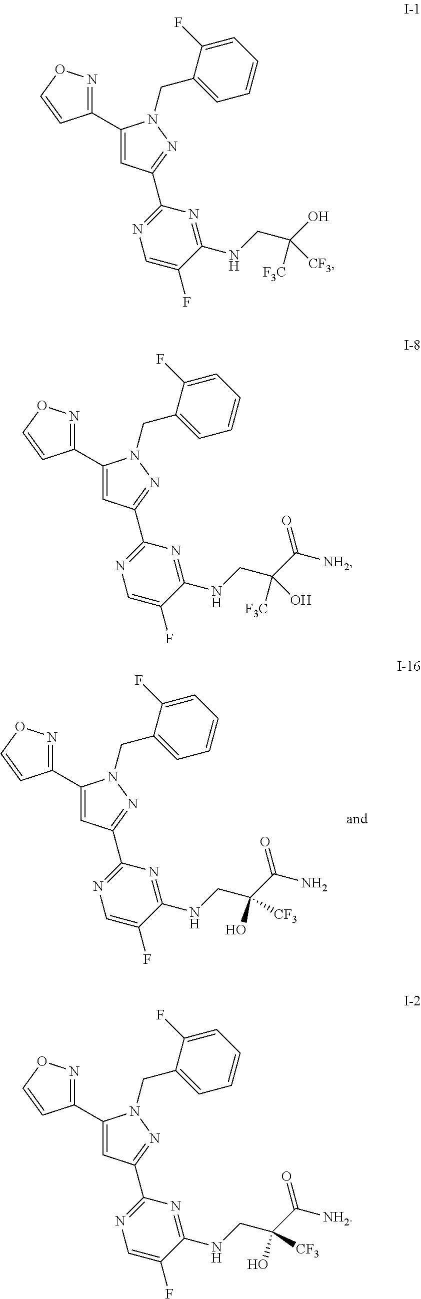 [SMILES: OC(CNc1nc(-c2cc(-c3ccon3)n(Cc3ccccc3F)n2)ncc1F)(C(F)(F)F)C(F)(F)F ; NC(=O)C(O)(CNc1nc(-c2cc(-c3ccon3)n(Cc3ccccc3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F]

In some embodiments, the compound of Formula I is selected from:

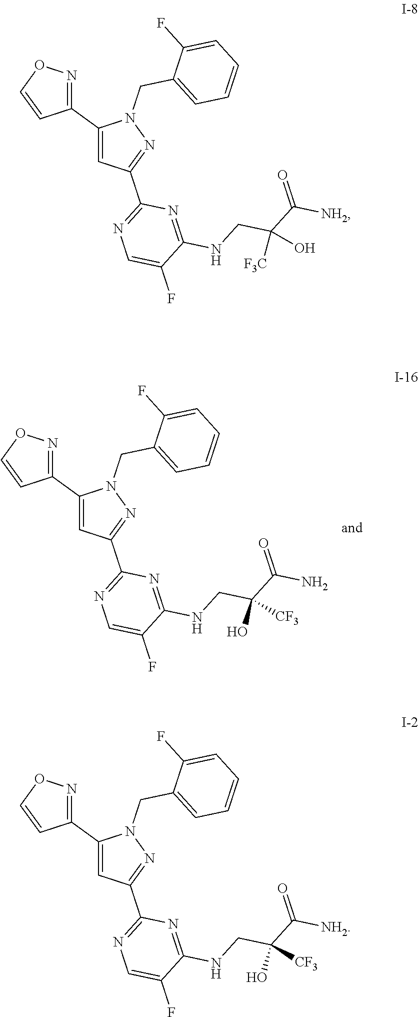 [SMILES: NC(=O)C(O)(CNc1nc(-c2cc(-c3ccon3)n(Cc3ccccc3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F]

In some embodiments of Formula I, the compound is Compound I-2. In other embodiments, the compound is Compound I-1. In still other embodiments, the compound is selected from Compound I-8 and Compound I-16.

In some embodiments of the first to thirteenth aspects of the invention, the sGC stimulator is a compound of Formula II:

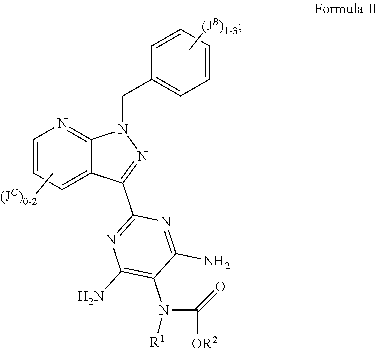

wherein:
each J^{B} is independently selected from halogen or C₁₋₃ alkyl;
each J^{C}, if present, is independently selected from halogen;
R¹ is hydrogen or C₁₋₃ alkyl; and
R² is a C₁₋₃ alkyl.

In some embodiments of Formula II, each J^{B} is a fluoro. In some embodiments of Formula II, at least one J^{B} is fluoro. In other embodiments, at least two J^{B} are fluoro.

In some embodiments of Formula II, one or two instances of J^{C} are present. In other embodiments, only one instance of J^{C} is present. In some of these embodiments, J^{C} is fluoro.

In some embodiments of Formula II, R¹ is selected from hydrogen, methyl or ethyl. In other embodiments, R¹ is hydrogen. In still other embodiments, R¹ is methyl.

In some embodiments of Formula II, R² is methyl or ethyl. In still other embodiments, R² is methyl.

In some embodiments of Formula II, the compound is vericiguat or riociguat, each depicted below and assigned labels Compound II-1 and Compound II-2, respectively.

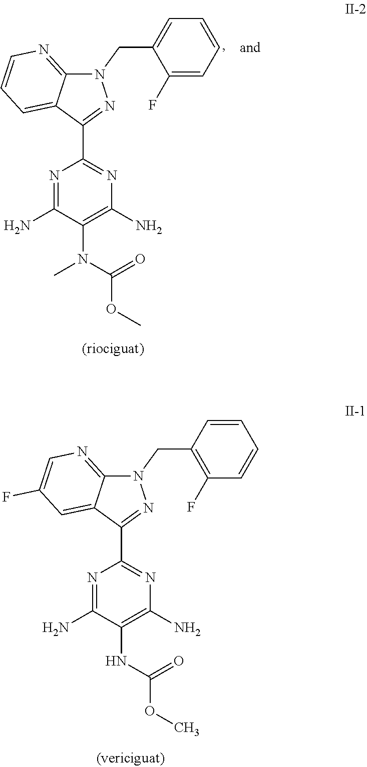 [SMILES: COC(=O)N(C)c1c(N)nc(-c2nn(Cc3ccccc3F)c3ncccc23)nc1N ; COC(=O)Nc1c(N)nc(-c2nn(Cc3ccccc3F)c3ncc(F)cc23)nc1N]

In some embodiments of Formula II, the compound is vericiguat. In other embodiments of Formula II, the compound is riociguat.

### Pharmaceutically Acceptable Salts

In addition to the compounds described herein, their pharmaceutically acceptable salts may also be employed for the methods and uses according to the first to thirteen embodiments and in compositions or dosage forms to treat the herein identified diseases, according to aspects one to thirteen of the invention.

A “pharmaceutically acceptable salt” of any of the compounds described herein include those derived from said compounds when mixed with inorganic or organic acids or bases. In some embodiments, the salts can be prepared in situ during the final isolation and purification of the compounds. In other embodiments the salts can be prepared from the free form of the compound in a separate synthetic step. The preparation of the pharmaceutically acceptable salts described above and other typical pharmaceutically acceptable salts is more fully described by BERG, et al. (Pharmaceutical Salts. J Pharm Sci. 1977:66:1-19), incorporated here by reference in its entirety. The pharmaceutically acceptable salts of an sGC stimulator, including, for example, compounds of Formula I or Formula II, are those that may be used in medicine. Salts that are not pharmaceutically acceptable may, however, be useful in the preparation of an sGC stimulator, including for example compounds of Formula I or Formula II or of their pharmaceutically acceptable salts.

When a compound is acidic, suitable “pharmaceutically acceptable salts” refers to salts prepared from pharmaceutically acceptable non-toxic bases, including inorganic and organic bases. Salts derived from inorganic bases include aluminum, ammonium, calcium, copper, ferric, ferrous, lithium, magnesium, manganic, manganous, potassium, sodium, zinc, and the like. Particular embodiments include ammonium, calcium, magnesium, potassium, and sodium salts. Salts derived from pharmaceutically acceptable organic non-toxic bases include salts of primary, secondary and tertiary amines, substituted amines including naturally occurring substituted amines, cyclic amines, arginine, betaine, caffeine, choline, N, N1-dibenzylethylenediamine, diethylamine, 2-diethylaminocthanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine, and the like.

In some embodiments, compounds of the invention have an acidic group that can react with a base (e.g., a pharmaceutically acceptable non-toxic base) to form a salt (e.g., a pharmaceutically acceptable salt). In some embodiments, the salt is an ammonium, calcium, magnesium, potassium, cessium or sodium salt. In other embodiments, the salt is a sodium salt.

When a compound is basic, salts may be prepared from pharmaceutically acceptable non-toxic acids, including inorganic and organic acids. Such acids include acetate, acetic, acid citrate, acid phosphate, ascorbate, benzenesulfonic, benzenesulfonate, benzoic, benzoate, bromide, bisulfate, bitartrate, camphorsulfonic, chloride, citrate, citric, ethanesulfonate, ethanesulfonic, formate, fumarate, fumaric, gentisinate, gluconate, gluconic, glucuronate, glutamate, glutamic, hydrobromic, hydrochloric, iodide, isethionic, isonicotinate, lactate, lactic, maleate, maleic, malic, mandelic, methanesulfonic, methanesulfonate, mucic, nitrate, nitric, oleate, oxalate, pamoic, pamoate (i.e., 1,1′-methylene-bis-(2-hydroxy-3-naphthoate)), pantothenic, pantothenate, phosphate, phosphoric, saccharate, salicylate, succinic, succinate, sulfuric, sulfate, tannate, tartrate, tartaric, p-toluenesulfonate, p-toluenesulfonic acid, and the like. Particular embodiments include citric, hydrobromic, hydrochloric, maleic, phosphoric, sulfuric, and tartaric acids.

### Pharmaceutical Compositions and Dosage Forms

The compounds herein disclosed, and their pharmaceutically acceptable salts thereof may be formulated as pharmaceutical compositions or “formulations” for the treatments and uses of the invention according to the first to thirteen aspects. These formulations may be manufactured as individual dosage forms.

A typical formulation is prepared by mixing a compound of the invention, or a pharmaceutically acceptable salt thereof, and a carrier, diluent, or excipient. Suitable carriers, diluents, and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which a compound of the invention is being formulated. Solvents are generally selected based on solvents recognized by persons skilled in the art as safe (i.e., GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc., and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow-release) preservatives, antioxidants, opaquing agents, glidants, processing aids, colorants, sweeteners, perfuming agents, flavoring agents, and other known additives to provide an elegant presentation of the drug (i.e., a compound of the invention or pharmaceutical composition thereof) or to aid in the manufacturing of the pharmaceutical product (i.e., medicament).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methyl methacrylate) microcapsules, respectively; in colloidal drug-delivery systems (for example, liposomes, albumin microspheres, microemulsions, nano-particles, and nanocapsules), or in macroemulsions. Such techniques are disclosed in Remington: The Science and Practice of Pharmacy (23rd Edition, University of the Sciences in Philadelphia, 2021.

The formulations may be prepared using conventional dissolution and mixing procedures. The term “therapeutically effective amount” as used herein means that amount of active compound or pharmaceutical agent that elicits the biological or medicinal response in a tissue, system, animal, or human that is being sought by a researcher, veterinarian, medical doctor, or other clinician. The therapeutically effective amount of the compound to be administered will be governed by such considerations, and is the minimum amount necessary to treat the disease or one or more of its symptoms.

The compositions described herein may be administered systemically or locally, e.g., orally (including, but not limited to solid dosage forms including hard or soft capsules [e.g., gelatin capsules], tablets, pills, powders, sublingual tablets, troches, lozenges, and granules; and liquid dosage forms including, but not limited to, pharmaceutically acceptable emulsions, microemulsions, aqueous or oil solutions, suspensions, syrups, and elixirs), by inhalation (e.g., with an aerosol, gas, inhaler, nebulizer, or the like), to the car (e.g., using car drops), topically (e.g., using creams, gels, inhalants, liniments, lotions, ointments, patches, pastes, powders, solutions, sprays, transdermal patches), ophthalmically (e.g., with eye drops, ophthalmic gels, ophthalmic ointments), rectally (e.g., using enemas or suppositories), nasally, buccally, vaginally (e.g., using douches, intrauterine devices, vaginal suppositories, vaginal rings or tablets, etc.), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally, or intravenously.

Formulations of a compound intended for oral use may be prepared according to any method known in the art for the manufacture of pharmaceutical compositions.

In solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, e.g., carboxymethylcellulose, alginates, gelatin, polyvinylpyrrolidone, sucrose, and acacia; c) humectants such as glycerol; d) disintegrating agents such as agar, calcium carbonate, potato or tapioca starch, alginic acid, certain silicates, and sodium carbonate; c) solution-retarding agents such as paraffin; f) absorption accelerators such as quaternary ammonium compounds; g) wetting agents such as, e.g., cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time-delay material such as glyceryl monostearate or glyceryl distearate alone or with a wax may be employed. A water soluble taste-masking material such as hydroxypropyl-methylcellulose or hydroxypropyl-cellulose may be employed.

In addition to the active compounds, liquid dosage forms may contain inert diluents commonly used in the art such as, e.g., water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Oral compositions (either solid or liquid) can also include excipients and adjuvants such as dispersing or wetting agents, such as a naturally occurring phosphatide (e.g., lecithin), a condensation product of an alkylene oxide with a fatty acid (e.g., polyoxyethylene stearate), a condensation product of ethylene oxide with a long-chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); emulsifying and suspending agents, such as sodium carboxymethylcellulose, croscarmellose, povidone, methylcellulose, hydroxypropyl methylcellulose, sodium alginate, polyvinylpyrrolidone, gum tragacanth and gum acacia; sweetening, flavoring, and perfuming agents; and/or one or more preservatives such as ethyl or n-propyl p-hydroxy-benzoate, one or more coloring agents, one or more flavoring agents and one or more sweetening agents, such as sucrose or saccharin.

The pharmaceutical compositions may also be administered by nasal aerosol or by inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The pharmaceutical compositions described herein may also be administered topically, especially when the target of treatment includes areas or organs readily accessible by topical application, including diseases of the eye, the car, the skin, or the lower intestinal tract. Suitable topical formulations are readily prepared for each of these areas or organs. The active component is admixed under sterile conditions with a pharmaceutically acceptable carrier and any needed preservatives or buffers as may be required.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax, and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol, and water.

Alternatively, the active ingredients may be formulated in a cream having an oil-in-water cream base. If desired, the aqueous phase of the cream base may include a polyhydric alcohol, i.e., an alcohol having two or more hydroxyl groups such as propylene glycol, butane 1,3-diol, mannitol, sorbitol, glycerol, and polyethylene glycol (including PEG 400), and mixtures thereof.

Topical formulations may desirably include a compound that enhances absorption or penetration of the active ingredient through the skin or other affected areas. Examples of such dermal penetration enhancers include dimethyl sulfoxide and related analogs.

The oily phase of emulsions prepared using a compound of the invention may be constituted from known ingredients in a known manner. While the phase may comprise merely an emulsifier (otherwise known as an emulgent), it desirably comprises a mixture of at least one emulsifier with a fat or an oil, or with both a fat and an oil. A hydrophilic emulsifier may be included together with a lipophilic emulsifier that acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base that forms the oily dispersed phase of the cream formulations. Emulgents and emulsion stabilizers suitable for use in the formulation of a compound of the invention include TWEEN® 60, Span® 80, cetostearyl alcohol, benzyl alcohol, myristyl alcohol, glyceryl mono-stearate, and sodium lauryl sulfate.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate-controlling membrane or by dispersing the compound in a polymer matrix or gel.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s). When formulated in an ointment, the active ingredients may be employed with either an oil-based, paraffinic, or water-miscible ointment base.

Compositions for rectal or vaginal administration are preferably suppositories that can be prepared by mixing the compounds described herein with suitable non-irritating excipients or carriers such as cocoa butter, beeswax, polyethylene glycol, or a suppository wax that are solid at ambient temperature but liquid at body temperature, and therefore melt in the rectum or vaginal cavity and release the active compound. Other formulations suitable for vaginal administration may be presented as pessaries, tampons, creams, gels, pastes, foams, or sprays.

Sterile injectable forms of the compositions described herein (e.g., for parenteral administration) may be aqueous or oleaginous suspension. These suspensions may be formulated according to techniques known in the art using suitable dispersing or wetting agents and suspending agents (including those described in the preceding paragraph). The sterile injectable preparation may also be a sterile injectable solution or suspension in a non-toxic parenterally-acceptable diluent or solvent, e.g., as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as a vegetable oil (e.g., arachis, olive, sesame, or coconut oil), especially in their polyoxyethylated versions, or in mineral oil such as liquid paraffin. These oil solutions or suspensions may also contain a long-chain alcohol diluent or dispersant, such as carboxymethyl cellulose or similar dispersing agents that are commonly used in the formulation of pharmaceutically acceptable dosage forms, including emulsions and suspensions. Other commonly used surfactants, such as Tweens, Spans, and other emulsifying agents or bioavailability enhancers that are commonly used in the manufacture of pharmaceutically acceptable solid, liquid, or other dosage forms may also be used for the purposes of injectable formulations. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

### EXAMPLES

For this invention to be better understood, the following examples are set forth. These examples are for purposes of illustration only and are not be construed as limiting the scope of the invention in any manner. All references provided in the Examples are herein incorporated by reference.

### Examples 1A and 1B

A Multicenter, Randomized, Double-Blind, Placebo-Controlled, Phase 2 Study Evaluating the Safety and Efficacy of Different Doses of Praliciguat (IW-1973, Compound I-1) Over 12 Weeks in Patients with Heart Failure with Preserved Ejection Fraction (CAPACITY HFpEF).

This trial started with the protocol described as in Example 1A and the protocol was later updated as described in Example 1B. Unless otherwise indicated in specific sections, the majority of the details described below apply to both versions of the protocol.

### List of Abbreviations and Definition of Terms

6MWT=6-minute walk test
ACE=angiotensin-converting enzyme
AE=adverse event
ANCOVA=analysis of covariance
ARB=angiotensin receptor blocker
ARNI=angiotensin receptor 1 antagonist/neprylisin inhibitor
AUC=area under the curve
BID=twice daily (2×/day)
BMI=body mass index (kg/m²)
BNP=B-type natriuretic peptide
BP=blood pressure
CSS=clinical summary score
cGMP=cyclic guanosine 3′,5′-monophosphate
CKD=chronic kidney disease
CPET=cardiopulmonary exercise test
CV=cardiovascular
CVD=cardiovascular disease
CYP3A=cytochrome P450 3A
DPP=dipeptidyl peptidase
ECG=electrocardiogram
E/e′=ratio mitral peak velocity [E]/mitral annulus early diastolic recoil velocity [e′]
EF=ejection fraction
eGFR=estimated glomerular filtration rate (mg/mL/1.73 m²)
EQ-5D=EuroQoL 5-dimension questionnaire
ERA=endothelin receptor antagonist
GLP=glucagon-like peptide
GTP=guanosine-5′-triphosphate
HF=heart failure
HFpEF=heart failure with preserved ejection fraction
HFrEF=heart failure with reduced ejection fraction
HOMA-IR=homeostatic model assessment to quantify insulin resistance
ICF=informed consent form
IV=intravenous(ly)
KCCQ=Kansas City Cardiomyopathy Questionnaire
kg=kilogram(s)
kg/m²=kilograms/meters squared [body mass index]
LA=left atrial
LSM=least-squares mean
LV=left ventricular
LVEF=left ventricular ejection fraction
mg=milligram(s)
mL=milliliter(s)
mmHg=millimeters of mercury
MRA=mineralocorticoid receptor antagonist
NAFLD=non-alcoholic fatty liver disease
NASH=non-alcoholic steatohepatitis
NO=nitric oxide
NT-proBNP=N-terminal pro B-type natriuretic peptide
NYHA=New York Heart Association
PAH=pulmonary arterial hypertension
PDE=phosphodiesterase
PEG=polyethylene glycol
PK=pharmacokinetic(s)
QD=once per day (1×/day)
QIDS-SR-16=Quick Inventory of Depressive Symptomatology Self-Report 16-item version
QOL=quality of life
RAAS=renin-angiotensin-aldosterone system
RER=respiratory exchange ratio
SAE=serious adverse event
SDS=Sheehan Disability Scale
sGC=soluble guanylate cyclase
SGLT2=sodium-glucose transport protein 2
T_{1/2}=apparent terminal elimination phase half-life
TEAE=treatment-emergent adverse event
UACR=urine albumin creatinine ratio
ULN=upper limit of normal [range]
US=United States
VO₂=oxygen consumption
VE/VCO₂ slope=minute ventilation/volume of carbon dioxide production

### Outcome Measures Studied

The primary objectives of this clinical study (ClinicalTrials.gov Identifier NCT03254485) were to assess the safety of oral praliciguat when administered for approximately 12 weeks to participants with HFpEF, and to evaluate the effect of oral praliciguat on peak exercise capacity (as measured by oxygen consumption [VO₂]) when administered for approximately 12 weeks to participants with HFpEF, both in all participants and in participants without permanent or persistent atrial fibrillation.

The primary safety endpoints were based on the incidence of treatment-emergent adverse events (TEAEs) and of TEAEs that were considered study drug-related by the investigator. TEAEs were defined as AEs that started or worsened in severity after administration of study drug. Medical history, prior and concomitant medications and surgeries/procedures, and adverse events (AEs) were collected throughout the study. Physical examination, vital sign measurements including orthostatic blood pressures, clinical laboratory tests, pregnancy tests, and ECGs were performed. Blood was also collected to determine pharmacokinetic (PK) parameters and plasma concentrations of praliciguat.

The primary efficacy endpoint was the change from baseline in peak VO₂ as measured by cardiopulmonary exercise testing (CPET) at Week 12 of the Treatment period. CPET was performed at baseline, at Week 8 and again at Week 12 using a highly standardized study-specific protocol. To participate in this study, each study site had to submit 2 studies to the CPET Core Laboratory at Massachusetts General Hospital core laboratory for review, and studies had to be determined to be of adequate quality before the site was deemed CPET qualified. For the 3 CPETs required by the study protocol, each study participant was to use the same form of exercise (treadmill or bicycle) and undergo the CPETs at the same time of day (+2 hours), ≥1 hour after study medication dosing. As part of the procedure, participants exercised to peak capacity beginning with unloaded exercise and continuing with a symptom-limited incremental ramp to peak VO₂ with requisite respiratory exchange ratio (RER)≥1. For the baseline CPET, a participant's RER had to be ≥1.0 in order to indicate an adequate test (i.e., adequate effort on the part of the study participant, to try to mitigate stopping for non-cardiopulmonary reasons [e.g., effort-related or musculoskeletal]). Additional CPET-related study eligibility criteria included a peak VO₂ of ≤80% of the predicted or age- and sex-adjusted normal level (FLETCHER G F, et al. Exercise standards. A statement for healthcare professionals from the American Heart Association. Writing Group. Circulation. 1995;91 [2]: 580-615), which served as an objective indicator of exercise intolerance characteristic of HF. For the Week 8 and End of Treatment CPETs, participants were to exercise to maximum effort, ideally to RER≥1.0. Analysis of CPET measurements for eligibility and for efficacy analyses were conducted by the independent central CPET Core Lab that was blinded to treatment allocation.

The secondary objective of this clinical study was to evaluate the effect of oral praliciguat on exercise and functional capacity when administered for approximately 12 weeks to participants with HFpEF.

Additional, exploratory, objectives of this clinical study were to explore the effect of oral praliciguat on heart function, biomarkers, quality of life (QOL), health status, and additional measures of exercise and functional capacity, when administered for approximately 12 weeks to participants with HFpEF, and to evaluate the pharmacokinetics (PK) of oral praliciguat when administered for approximately 12 weeks to participants with HFpEF.

The pre-specified major secondary endpoints were the change from baseline in 6-minute walk test (6MWT) distance at Week 12, the change from baseline in ventilatory efficiency as defined by VE/VCO₂ slope (minute ventilation/volume of carbon dioxide production) as obtained via CPET at Week 12, and the number of CPET responders defined as participants who improved by at least 1.5 mL O₂/kg/min in peak VO₂ from baseline to Week 12. In the CPET responder analysis, participants who were hospitalized or who died due to heart failure during the study Treatment Period or were missing Week 12 post-dose CPET values were considered to be non-responders.

Other exploratory endpoints included assessment of echocardiographic parameters (as determined by an independent, central echocardiography core laboratory that was blinded to treatment assignment); changes from baseline to Week 12 and Follow-up visits in New York Heart Association (NYHA) functional classification (I, II, III, or IV); cardiac events (hospitalizations of CV etiology and all deaths were analyzed by the central, independent, blinded cardiac events adjudicators who determined if an event was due to heart failure or was of other CV etiology); changes from baseline to Week 12 in blood and urine biomarker levels that are relevant to HF pathophysiology; and changes from baseline to Week 12 in NT-proBNP values, urine albumin creatinine ratios (UACRs), estimated glomerular filtration rates (eGFRs), and HOMA-IR changes. Changes from baseline responses in participants' self-administered health-related quality-of-life (QOL) questionnaires were also tabulated and analyzed, including the Kansas City Cardiomyopathy Questionnaire (KCCQ), the Sheehan Disability Scale (SDS), the EuroQol (EQ) 5D-5L, and the Quick Inventory of Depressive Symptomatology Self-Report 16-item version (QIDS-SR-16).

The overall pre-specified success criteria for the efficacy endpoints were summarized for this study as shown below in Table 1.

TABLE 1
pre-specified success assessments

| Endpoint | Clinically Significant Improvement | | “Unequivocal” improvement | |
|---|---|---|---|---|
| CPET | | | | |
| Peak VO, | 1.0 mL O₂/kg/min | | 1.5 mL O₂/kg/min | |
| VE/VCO, slope | 5-10% | | >10% | |
| Work Load | 5-10% | | >10% | |
| 6-minute walk | 15 | meters | 30 | meters |
| KCCQ Physical | 5 | points | >10 | points |
| Limitation Score | | | | |

### Study Design: Population Studied

This Phase 2, multicenter, randomized, double-blind, placebo-controlled, parallel-group study evaluated the safety and efficacy of praliciguat compared with placebo. The study randomized 196 adults with established heart failure (HF) and limited exercise capacity with an ejection fraction (EF) of at least 40% and who had at least 2 of 4 risk factors for HFpEF (diabetes/prediabetes, hypertension, obesity, and advanced age) (see Inclusion and Exclusion Criteria below).

### Example 1A

Participants were randomized to 3 dose levels of praliciguat or placebo and received study drug for up to 89 days. On Day 1, participants were randomized in a 1:1:1:1 ratio to the following 4 regimens:

Treatment Weeks 1 and 2: twice daily (BID) dosing; Treatment Weeks 3 through 12: once daily (QD) dosing

10 mg total daily dose: one 5-mg praliciguat tablet, orally BID; then two 5-mg praliciguat tablets, orally QD.

20 mg total daily dose: one 10-mg praliciguat tablet, orally BID; then two 10-mg praliciguat tablets, orally QD.

40 mg total daily dose: one 20-mg praliciguat tablet, orally BID; then two 20-mg praliciguat tablets, orally QD.

Placebo: one matching placebo tablet, orally BID; two matching placebo tablets, orally QD.

At the Day 1 Visit, eligible participants were stratified by atrial fibrillation status and by baseline peak VO₂ (<60% or ≥60% of age- and sex-adjusted normal values and with RER≥1.0 as determined by CPET). The number of participants admitted with permanent or persistent atrial fibrillation was limited to approximately 20% of the total enrollment per treatment arm.

The study consisted of 3 distinct periods, a Screening Period, a Treatment Period, and a Follow-up Period (Scheme 1).

Screening Period: The Screening Period began with the signing of the informed consent form (ICF) at the Screening Visit and lasted up to 45 days. At the Screening Visit, participants underwent screening procedures to determine their initial study eligibility. At the Baseline Visit, participants returned to the clinic for additional screening procedures to confirm their eligibility, including assessment of their CPET values, vital sign measurements, and blood and urine sample collections, and to receive urine sample collection supplies to take home. The Screening Visit could be combined with the Baseline Visit if the participant was fasted for blood and urine collections. The CPET and 6-minute walk test (6MWT) occurred within 14 days before Randomization. The end of the Screening Period coincided with the beginning of the Treatment Period.

Treatment Period: The Treatment Period began on Day 1 at Randomization and ended after the End of Treatment Visit on Day 85 (−7/+4 days). At the Day 1 Visit, eligible participants were stratified as described in and randomized 1:1:1:1 to daily 10 mg praliciguat, 20 mg praliciguat, 40 mg praliciguat, or placebo for approximately 12 weeks. Dosing on Days 1 to 14 (+3) was BID (twice daily) and dosing on Day 15 (+3) onward was QD (once daily).

At the Randomization (Day 1) Visit, eligible participants received their morning BID dose of study drug in the clinic and then self-administered the remainder of the BID doses through Day 14 at home. At the Week 2 Visit (Day 15 [+3]), participants returned to the clinic and received their first QD dose of study drug in the clinic. At both visits, participants remained in the clinic for at least 6 hours postdose to undergo safety, efficacy, and PK assessments, including blood and urine collections at prespecified times, and thereafter were allowed to leave the clinic at the Investigator's discretion. They were issued study drug supply to self-administer at home each day until their next scheduled visit.

At the Week 4 Visit, Week 8 Visit, and End of Treatment Visit, participants returned to the clinic for study drug administration; safety, efficacy, and PK assessments; and study drug and other supplies, as applicable.

Follow-up Period: The Follow-up Period began immediately after the End of Treatment Visit on Day 85 (−7/+4 days) and lasted for 28 (+7) days. On Day 113 (+7 days), participants returned to the clinic for the final Follow-up Visit.

### Example 1B

Participants were randomized in a 1:1 ratio to receive daily praliciguat or placebo as described below for up to 12 weeks. The number of participants admitted with permanent or persistent atrial fibrillation was limited to under 20% of total target enrollment.

Dosing on Days 1 to 14 (±3) was 20 mg BID (twice daily). Dosing on Day 15 (±3) onward was 40 mg QD (once daily). Participants received their first morning BID dose in the clinic on Day 1 and their first QD dose at the Week 2 Visit (Day 15 [+3]).

The study consisted of 3 distinct periods, a Screening Period, a Treatment Period, and a Follow-up Period (Scheme 2).

Screening Period: The Screening Period started with the signing of the ICF at the Screening Visit and lasted up to 45 days. At the Screening Visit, potential participants underwent screening procedures to determine their initial study eligibility. At the Baseline Visit, participants returned to the clinic for additional screening procedures to confirm their eligibility, including assessment of their CPET values, vital sign measurements, and blood and urine sample collections, and to receive urine sample collection supplies to take home. The Screening Visit could be combined with the Baseline Visit if the participant was fasted for blood and urine collections. CPET and 6MWT were to occur within 14 days before Randomization. The end of the Screening Period coincided with the beginning of the Treatment Period.

Treatment Period: The Treatment Period began on Day 1 at Randomization and ended after the End of Treatment Visit on Day 85 (−7/+4 days). At the Day 1 Visit, eligible participants were stratified and randomized as described above. Dosing on Days 1 to 14 (±3) was 20 mg BID (twice daily) and dosing on Day 15 (±3) onward was 40 mg QD (once daily).

At the Randomization (Day 1) Visit, participants received their morning BID dose of study drug in the clinic. At the Week 2 Visit (Day 15 [±3]), participants returned to the clinic and received their first QD dose of study drug in the clinic. At both visits, participants stayed in the clinic a minimum of approximately 4 hours postdose to undergo safety, efficacy, and PK assessments, including blood and urine collections at prespecified times, and thereafter were allowed to leave the clinic at the Investigator's discretion. They were issued a supply of study drug to self-administer each day at home until their next scheduled visit.

At the Week 4 Visit, Week 8 Visit, and End of Treatment Visit, participants returned to the clinic for study drug administration; safety, efficacy, and PK assessments; and study drug and other supplies, as applicable.

Follow-up Period: The Follow-up Period began immediately after the End of Treatment Visit on Day 85 (−7/+4 days) and lasted for 28 (±7) days. On Day 113 (±7 days), participants returned to the clinic for the final Follow-up Visit.

### Stopping Criteria: Examples 1A and 1B

On an individual basis, a participant was discontinued from study drug dosing if any of the following were reported: a study drug-related serious adverse event (SAE) of spontaneous bleeding; a study drug-related life-threatening symptomatic hypotension-related SAE; or 2 study drug-related symptomatic hypotension-related SAEs.

### Dosage Regimens:

### Example 1A

10 mg, 20 mg or 40 mg total oral daily dose as one dose QD or half-dose BID.

### Example 1B

40 mg total oral daily dose as 40 mg QD or 20 mg BID.

### Study Drug:

### Example 1A

Compound I-1 was administered as multiples of a 5-mg oral tablet dosage form (10-mg dose), as multiples of a 10-mg oral tablet dosage form (20-mg dose) or multiples of a 20-mg oral tablet dosage form (40 mg dose). Placebo was administered as multiples of a matching placebo tablet. Compound I-1 was formulated as a spray-dried dispersion formulation as described in WO2017095697.

### Example 1B

Compound I-1 was administered as multiples of a 20-mg oral tablet dosage form. Placebo was administered as multiples of a matching placebo tablet. Compound I-1 was formulated as a spray-dried dispersion formulation as described in WO2017095697.

### Study Drug Administration: Example 1A and Example 1B

During Treatment Weeks 1 and 2, participants were instructed to take study drug twice daily (BID), as 1 tablet in the morning and 1 tablet approximately 12 hours later in the evening, at approximately the same time each day. Administration of split doses during the first two weeks of dosing was expected to reduce the initial time to maximum observed plasma concentration and to minimize the variations in Compound I-1 plasma concentration during the approach to steady state, which may improve tolerability. Starting with the Week 2 Visit (Day 15±3), participants were instructed to take the study drug once daily (QD), as 2 tablets in the morning, preferably at approximately the same time each day.

Dose reduction was allowed on a per-participant basis at the site investigator's discretion after consultation with the study's Medical Monitor. If dose reduction was approved, the participant was allowed to reduce his or her daily dose by half, i.e., from two tablets daily to one tablet daily (in the morning). Each participant's dose was only allowed to be reduced once; increase was not allowed after reduction.

### Inclusion Criteria:

### Example 1A

Participant was an ambulatory male or female ≥50 years old at the Screening Visit.

Participant had heart failure with ejection fraction (EF) of ≥45% as assessed within 12 months of the Screening Visit, without previously documented EF of <40%.

### Example 1B

Participant was an ambulatory male or female ≥45 years old at the Screening Visit.

Participant had heart failure with ejection fraction (EF) of ≥40% as assessed within 12 months of the Screening Visit, without previously documented EF of <40%.

### Examples 1A and 1B

The following inclusion criteria applied to both the initial design of the trial described in Example 1A and to the update reflected in Example 1B:

Participant had a peak VO₂ measuring <80% of age- and sex-adjusted normal (see Table 2 below) and a respiratory exchange ratio (RER)≥1.0 at the Baseline Visit cardiopulmonary exercise test (CPET) as determined by the CPET Core Lab.

TABLE 2
Age- and Sex-adjusted Normal Peak VO₂

| Age | | Normal Peak VO₂ | 80% of Normal Peak VO₂ |
|---|---|---|---|
| (years) | Sex | (mL O₂/kg/min) | (mL O₂/kg/min) |
| 40-49 | M | 32 | 25.6 |
| 40-49 | F | 40 | 32.0 |
| 50-59 | M | 36 | 28.8 |
| 50-59 | F | 29 | 23.2 |
| 60-69 | M | 33 | 26.4 |
| 60-69 | F | 27 | 21.6 |
| >70 | M | 29 | 23.2 |
| >70 | F | 27 | 21.6 |

1. Source: Fletcher et al, 1995
2. F = female; M = male

Participant may have had permanent or persistent atrial fibrillation; the total number of participants with permanent or persistent atrial fibrillation was limited to approximately 20% of the total number of participants. (Note: Participants with intermittent [paroxysmal] atrial fibrillation had to be in normal sinus rhythm at the time of the baseline CPET; these participants did not count toward the limit of 20% of participants with permanent or persistent atrial fibrillation.)

Participant had evidence in medical history supporting clinical heart failure syndrome consisting of at least 1 of the following:
a. Hospitalization or emergency department visit for heart failure within the past year, with at least 2 of the following documented:
   Volume overload on presentation as evidenced by at least 2 of the following signs: jugular venous distension, pitting edema ≥1+, ascites, pulmonary, congestion on chest x-ray, or pulmonary rales
   B-type natriuretic peptide (BNP) (≥100 [sinus rhythm], ≥200 pg/mL [atrial fibrillation]) or N-terminal pro BNP (NT-proBNP) (>300 [sinus rhythm] or ≥600 pg/mL [atrial fibrillation])
   Treatment with intravenous (IV) diuretics with clinical response
b. BNP (≥100 [sinus rhythm], ≥200 pg/mL [atrial fibrillation]) or NT-proBNP (>300 [sinus rhythm], >600 pg/mL [atrial fibrillation]) within the past 6 months. (If needed for eligibility, a sample for NT-proBNP level could be collected at the Screening Visit.)
c. Echocardiographic evidence within the past 12 months of at least 2 of the following: left ventricular (LV) hypertrophy, left atrial (LA) enlargement, or diastolic dysfunction (medial mitral peak velocity/mitral annulus early diastolic recoil velocity [E/e′ ratio]≥15)
d. Hemodynamic evidence of elevated filling pressures, as indicated by pulmonary capillary wedge pressure ≥15 mmHg at rest or >25 mmHg with exercise within the past 12 months, or left ventricular end-diastolic pressure ≥15 mmHg

Participant had New York Heart Association (NYHA) Class II-IV symptomatology as assessed at the time of the Screening Visit.

Participant was on stable dose(s) of any current cardiovascular medication (e.g, angiotensin-converting enzyme [ACE] inhibitors, angiotensin receptor blockers [ARBs], beta-blockers, mineralocorticoid receptor antagonists [MRAs]) for at least 1 month before the Baseline and Randomization Visits, with the regimen expected to remain unchanged for the duration of the trial. Diuretic doses did not need to be stable.

Participant met at least 2 of the following criteria at the Screening Visit:
a. Diagnosis of type 2 diabetes mellitus or prediabetes (currently treated or hemoglobin A1c≥5.6)
b. History of hypertension (on at least 1 antihypertensive medication or had current seated blood pressure [BP]>140/90 mmHg)
c. Body mass index (BMI)>30 kg/m²
d. Age ≥70 years

Female participant was postmenopausal (no menses for ≥12 consecutive months), or surgically sterile (i.e., bilateral oophorectomy, hysterectomy, or tubal sterilization [tie, clip, band, or burn]), or if of reproductive potential, agreed to completely abstain from heterosexual intercourse; or, if heterosexually active and of reproductive potential, agreed to use 1 of the protocol-specified methods of birth control from the date she signed the ICF until 60 days after her final dose of study drug.

Male participant who was not surgically sterile by vasectomy (conducted ≥60 days before the Screening Visit or confirmed via sperm analysis) agreed to completely abstain from heterosexual intercourse or, if heterosexually active, agreed to use a highly effective birth control method suggested in the protocol starting from the Screening Visit through 60 days after his final dose of study drug.

Participant agreed not to make any major lifestyle (e.g., diet, exercise) changes from the Screening Visit through the Follow-up Visit.

### Exclusion Criteria (Examples 1A and 1B):

Participants who met any of the following criteria were not eligible to participate in the study:

Participant had acute coronary syndrome or percutaneous coronary intervention within 30 days before Randomization.

Participant had had cardiac transplantation or had it planned during the study.

Participant had had coronary artery bypass graft, cardiac mechanical support implantation, or other cardiac surgery in the 3 months before the Screening Visit or had it planned during the study.

Participant had severe chronic obstructive pulmonary disease (COPD) as defined by chronic oxygen dependence. Nighttime oxygen was not exclusionary.

Participant had a heart failure hospitalization with discharge within 30 days before the Baseline Visit.

Participant had hypertrophic cardiomyopathy (obstructive or nonobstructive), restrictive cardiomyopathy, active myocarditis, constrictive pericarditis, cardiac sarcoidosis, or known amyloid cardiomyopathy.

Participant had uncontrolled arrhythmias other than permanent or persistent atrial fibrillation.

Participant had history of uncorrected congenital cardiac disease affecting LV function.

Participant has evidence of severe chronotropic incompetence, as indicated by a heart rate response <55% of predicted maximum heart rate (220-age) per Baseline Visit CPET.

Participant had any history of platelet dysfunction, hemophilia, von Willebrand disease, coagulation disorder causing a bleeding diathesis, other bleeding diathesis, or significant, nontraumatic bleeding episodes, such as from a gastrointestinal source.

Participant had uncorrected thyroid disease.

Participant had severe aortic stenosis or severe mitral regurgitation.

Participant had seated systolic BP<110 mmHg at the Screening Visit and on Day 1 before Randomization. For these determinations of eligibility, BP was average of 3 measurements obtained at approximately 2-minute intervals after the participant had been sitting quietly for ≥5 minutes.

Participant had estimated glomerular filtration rate (eGFR)

<30 mL/min/1.73 m² at the Screening Visit and/or was receiving dialysis.

Participant had direct bilirubin >2 times upper limit of normal (×ULN, as defined by laboratory) or alanine aminotransferase or aspartate aminotransferase >3×ULN at the Screening Visit.

Participant received IV inotropic therapy within 30 days before the Screening Visit.

Participant was unable to take oral medications.

Participant had active or treated malignancies within 12 months of the Screening Visit, except for basal cell carcinoma.

Participant had previously received praliciguat or received any other investigational drug during the 30 days or 5 half-lives of that investigational drug (whichever was longer) before the Screening Visit, was planning to receive another investigational drug at any time during the study, had an active investigational medical device currently implanted and/or was planning to have one implanted at any time during the study.

Participant had a comorbid condition with an expected survival less than 6 months.

Participant had any medical condition that, in the Investigator's opinion, could lead to difficulty complying with protocol procedures (e.g., 6MWT, CPET) or could prohibit completion of the study. Specifically, participant was not primarily limited in their physical activity by joint, leg, hip or back pain, or gait unsteadiness.

Participant had evidence of active hepatitis or human immunodeficiency virus antibody at the Screening Visit.

Participant had clinically significant (per Investigator judgment) history of viral or bacterial infection within 4 weeks of the Baseline Visit.

Participant had surgery with general anesthesia in the 6 weeks before the Screening Visit or had scheduled or planned surgery with general anesthesia during the study.

Participant had a history of active alcoholism or drug addiction during the 12 months before the Screening Visit and/or had a positive drug screen at the Screening Visit for amphetamines, barbiturates, benzodiazepines, cocaine, opiates, phencyclidine, propoxyphene without a prescription for a medically defined condition.

Participant was taking specific inhibitors of phosphodiesterase 5 (PDE5, including sildenafil and tadalafil), nonspecific inhibitors of PDE5 (including dipyridamole and theophylline), any supplement for the treatment of erectile dysfunction, riociguat or any other sGC stimulator, and/or nitrates or nitric oxide (NO) donors in any form. These medications and supplements were prohibited from 7 days before Randomization through the duration of the study.

Participant was taking strong cytochrome P450 3A (CYP3A) inhibitors, examples of which include azole antifungals, macrolide antibiotics, protease inhibitors, and diltiazem. These medications and excessive grapefruit intake were prohibited 14 days before randomization through the duration of the trial.

Participant had a history of clinically significant hypersensitivity or allergy to any of the ingredients contained in the active or placebo drug products.

Female participant was pregnant (positive urine pregnancy test) breastfeeding. Breastfeeding was not allowed from Screening Visit through 60 days after the final dose of study drug.

Female participant who wished to become pregnant and/or planned to undergo egg donation or egg harvesting for current or future in vitro fertilization during the study and/or within at least 60 days after the final dose of study drug.

Male participant unwilling to refrain from sperm donation during the study and for at least 60 days after the final dose of study drug.

Participant would be unable to adhere to the trial assessment schedule or, in the clinical judgment of the Investigator, was otherwise not suitable for the trial.

### Top Line Assessments/Results

The analysis of the full set of results of this trial has been published, see: UDELSON J. E., et al. Effect of praliciguat on peak rate of oxygen consumption in patients with heart failure with preserved ejection fraction: the CAPACITY HFpEF randomized clinical trial. JAMA. 2020;324 (15): 1522-31. doi: 10.1001/jama.2020.16641.

A total of 196 participants (“Intention to Treat Population” or ITT) were randomized to one of three praliciguat dosing regimens or to placebo. Early on, the study was refocused to randomize participants into only the 40-mg praliciguat regimen (n=92 participants) or placebo (n=90 participants), and the lower praliciguat dosing regimens were discontinued. Among those randomized and dosed in the 40 mg praliciguat or placebo regimen (mean [SD] age, 70 [9] years; 75 [41%] women), 155 (86%) completed the trial. A total of 143 participants in the placebo and 40-mg praliciguat groups who completed the trial were included in the prespecified “Primary Analytic Population”, which was defined as those participants who had taken the assigned study drug or placebo for at least 8 weeks, had ≥1 evaluable post-baseline assessment, and did not have any dose reduction or major protocol deviations. In the placebo (n=78) and 40-mg praliciguat (n=65) groups of this population, adjusted changes in peak VO₂ were 0.04 mL/kg/min (95% CI, −0.49 to 0.56) and −0.26 mL/kg/min (95% CI, −0.83 to 0.31), respectively; the placebo-adjusted least-squares mean (LSM) difference in change from baseline was −0.30 mL/kg/min (95% CI, −0.95 to 0.35; P=0.37).

Most TEAEs reported in the study were considered to be mild or moderate in severity by the investigator. TEAEs considered to be related to study drug. TEAEs leading to discontinuation of study drug (AEDCs), and AECIs were reported at a higher incidence in participants treated with 40 mg praliciguat compared with placebo. The study drug-related TEAEs that occurred more often in the 40-mg placebo group vs. placebo were headache (11% vs. 7%), dizziness (10% vs. 1%), and hypotension (9% vs. 0%). Treatment-emergent SAEs were reported in 19 participants: 10 (11%) participants who received 40 mg praliciguat and 9 (10%) who received placebo. One SAE, reported in a participant who received placebo, was considered related to study drug by the Investigator. One death was reported in a participant who received 40 mg praliciguat; the contributing fatal events were considered not related to study drug by the Investigator.

None of the results for the 3 prespecified secondary endpoints were statistically significant when the overall Primary Analytic Population was analyzed. In the placebo and praliciguat groups, adjusted changes in 6-minute walk test (6MWT) distance were 58.1 meters (m) (95% CI, 26.1-90.1) and 41.4 m (95% CI, 8.2-74.5), respectively; the placebo-adjusted LSM difference in change from baseline was −16.7 m (95% CI, −47.4 to 13.9). In the placebo and praliciguat groups, the placebo-adjusted LSM difference in change in ventilation/carbon dioxide production slope was −0.3 (95% CI, −1.6 to 1.0). Thus, among this mixed-sex and -age population with HFpEF, the sGC stimulator praliciguat, compared with placebo, did not significantly improve peak VO₂ from baseline to Week 12 or improve any of the other secondary endpoints when compared with placebo. As a result, it was initially concluded that these findings did not support the use of praliciguat in patients with HFpEF and the development of praliciguat for HFpEF was halted.

### Primary Analytical Population: Age and Sex Distribution

The distribution of the Primary Analytical Population by sex and age is summarized below (Table 3):

TABLE 3
population distribution

| Characteristic | Placebo | Praliciguat 40 mg |
|---|---|---|
| Statistic/Category | (N = 78) | (N = 65) |
| Age Category, n (%) | | |
| <=70 years | 39 (50.0) | 25 (38.5) |
| >70 years | 39 (50.0) | 40 (61.5) |
| Sex, n (%) | | |
| Male | 43 (55.1) | 39 (60.0) |
| Female | 35 (44.9) | 26 (40.0) |
| One Year Post Menopausal | 23 (29.5) | 15 (23.1) |
| Surgically Sterile | 12 (15.4) | 11 (16.9) |
| Age and Sex Category, n (%) | | |
| Male <= 70 years | 22 (28.2) | 15 (23.1) |
| Male >70 years | 21 (26.9) | 24 (36.5) |
| Female <= 70 years | 17 (21.8) | 10 (15.4) |
| Female >70 years | 18 (23.1) | 16 (24.6) |

It is worth noting that in the general HFpEF population around 60% are female, whereas this CAPACITY HFpEF trial of praliciguat, only about 40% of the participants in the Primary Analytical Population were women.

### Secondary, Exploratory, Post-Hoc Sub-Population Analysis in Elderly Women

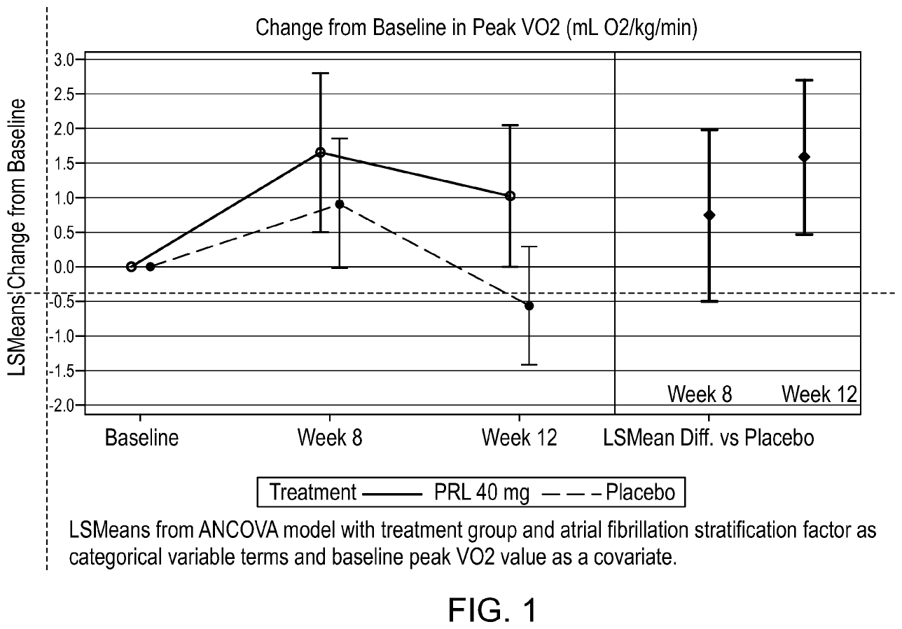

In an exploratory post hoc analysis of a sub-population of elderly (ages 70 years or older) female participants in the CAPACITY HFpEF study, those who received 40 mg praliciguat (n=16) were shown to have an increase from their baseline peak VO₂ values by Treatment Week 8 compared with those who received placebo (n=18), and the improvement was statistically significant (nominal P<0.05; p=0.007) by Week 12; results were based on estimates from an ANCOVA model with treatment group and atrial fibrillation stratification factor as categorical variable terms and baseline peak VO₂ as a covariate. See FIG. 1. In addition, the mean improvement in peak VO₂ as shown in FIG. 1 at Week 12 indicates an unequivocal improvement in this parameter based on the predefined “success” criteria for this study summarized in Table 1 above. At Week 8: the placebo-adjusted least-squares mean (LSM) difference in change from baseline was 0.75 ml/kg/min (95% CI, −0.49 to 1.98; P=0.23) and at Week 12: the placebo-adjusted least-squares mean (LSM) difference in change from baseline was 1.58 mL/kg/min (95% CI, 0.47 to 2.69; nominal p=0.007).

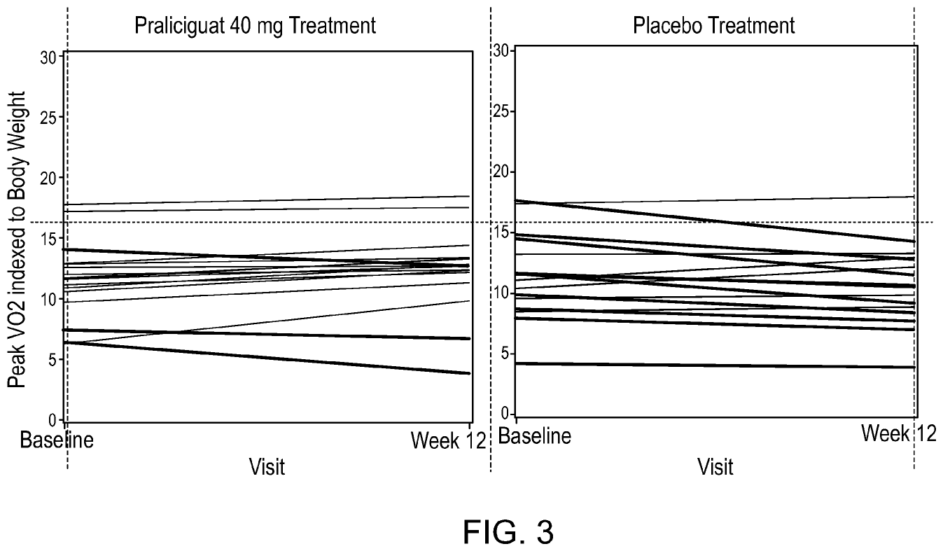

On an individual participant basis, a trend towards improvement in peak VO₂ was evident across the 12-week treatment period in the elderly women who received 40 mg praliciguat compared with those who received placebo; see FIG. 3.

Additional efficacy endpoint parameter results were also analyzed for this small sub-population (i.e., VE/VCO₂ slope, peak workload, 6MWT, and KCCQ-physical limitation score). Although these data reflect baseline variability that is inherent in posthoc subgroup analyses, and the results did not reach clinical or statistical significance, positive trends were observed in 6MW and peak workload % improvement, that were clearly larger than those of the general population and within the pre-defined success criteria compiled above. The improvement in peak work load from Table 4, below represents an improvement of about 6.5% (success criteria 5-10%). In comparison, this value for the whole population had a worsening of 1%. For the subpopulation of elderly women an improvement in 6MW of 23 meters (success criteria more than 15 m) was measured vs placebo, whereas for the overall population a worsening of 17 feet was observed relative to placebo.

TABLE 4
Analysis of Secondary Efficacy Endpoints in Sub-
Population of Women of Age > 70 at week 12

| Parameter | Statistics | | Praliciguat 40 mg |
|---|---|---|---|
| Six Minute | LS Mean | 50.8836 | 73.6369 |
| Walk Test | 95% LS Mean CI | −2.7664, | 13.0844, |
| | | 108.5336 | 134.1893 |
| | LS Mean Diff | | 22.7533 |
| | 95% LS Mean Diff CI | | −35,6906, |
| | | | 81.1971 |
| | p-value Biff | | 0.4314 |
| Peak | LS Mean | 5.7455 | 12.6395 |
| Workload | 95% LS Mean CI | −9.8731, | −5.3462, |
| | | 24.0707 | 34.0428 |
| (Watts) | LS Mean Diff | | 6.5195 |
| | 95% LS Mean Diff CI | | −10.2262, |
| | | | 26.3888 |
| | P-value Diff | | 0.4552 |

There were no statistically significant improvements suggested for cardiometabolic function endpoints in this sub-population at Week 12, However, relevant benefits were observed with respect to reduction of systolic and diastolic blood pressure as well as HbA1c (Table 5). Whereas the change from baseline relative to placebo in the general population was a reduction of 3.5 mmHg in the systolic blood pressure, this change was 7.9 mmHg reduction for the subpopulation of elderly women. Similarly, whereas the change from baseline relative to placebo in the general population was a reduction of 2.1 mmHg in the diastolic blood pressure, this change was 8.2 mmHg reduction for the subpopulation of elderly women. And also similarly, the change from baseline relative to placebo in the general population was a reduction of 0.18% points in the measurement of HbA1C, this change was 0.41% reduction from baseline relative to placebo for the subpopulation of elderly women

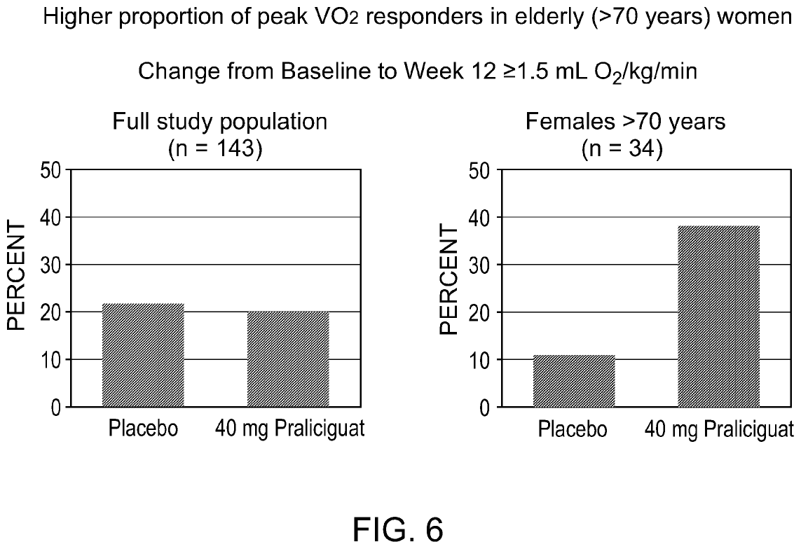

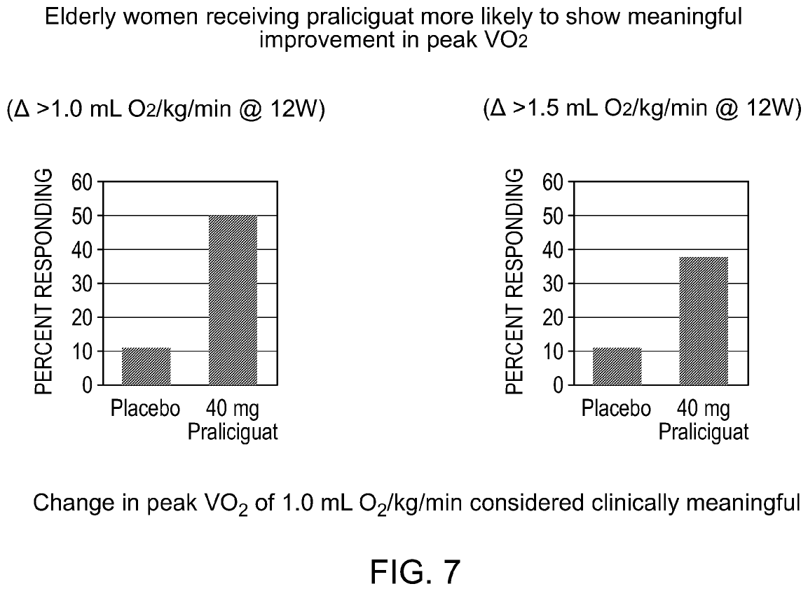

In the CPET responder analysis, a higher proportion of peak VO₂ responders was found among the population of elderly (older than 70 yold) women that among the full population. See FIG. 6. About 50% of patients in this sub-population showed a higher than 1 mL/02/kg/min improvement in peak VO₂ at 12 weeks of treatment, compared with only about 10% in the placebo arm. Furthermore, about 35-40% of patients in this sub-population showed a higher than 1.5 mL/O₂/kg/min improvement in peak VO₂ at 12 weeks of treatment, compared with only about 10% in the placebo arm. See FIG. 7.

Results of this analysis in this small, underrepresented sub-population of the CAPACITY HFpEF study suggest that sGC stimulators may be more useful in HFpEF patients that are post-menopausal women than in other populations and warrant further investigation.

### Example 2: Olinciguat (Compound I-2) Preclinical Data

Although olinciguat has not been studied in clinical trials of heart disease, pre-clinical data suggests that it could show similar results to those observed with praliciguat in post-menopausal women with HFpEF.

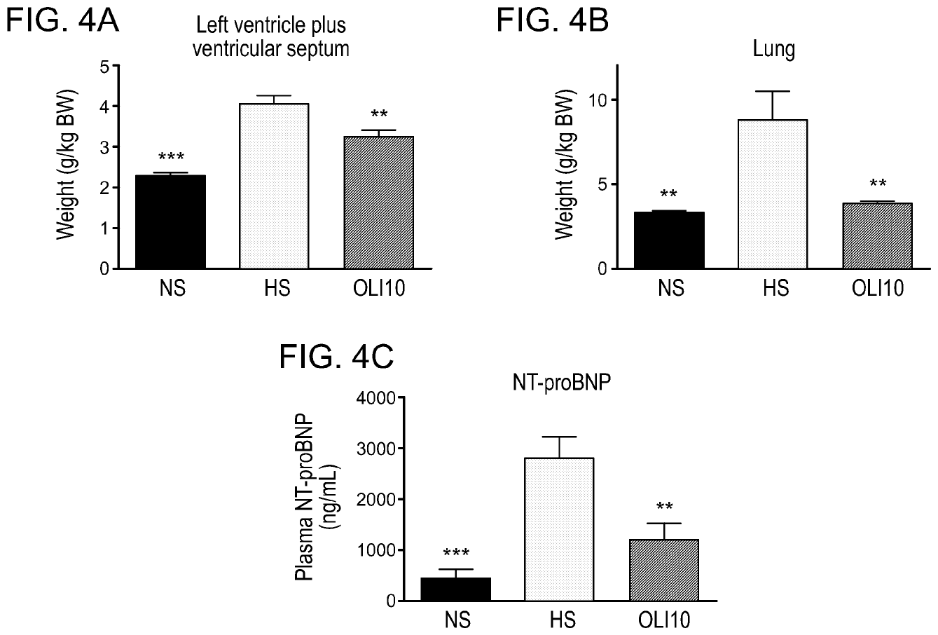

The cardioprotective effects of olinciguat were tested in two animal models of HF: the rat post-myocardial infarction (MI) model of heart failure with reduced ejection fraction and in the Dahl salt-sensitive (DSS) hypertension model, which develops HFpEF. Olinciguat was explored in rodent models to study its effects on the vasculature, the heart, the kidneys, metabolism, and inflammation (For example see ZIMMER D P, et al., Olinciguat, an oral sGC stimulator, exhibits diverse pharmacology across preclinical models of cardiovascular, metabolic, renal, and inflammatory disease. Front. Pharmacol. 2020, 11:419. doi: 10.3389/fphar.2020.00419). Olinciguat reduced blood pressure in normotensive and hypertensive rats. Olinciguat was cardioprotective, reduced lung congestion and reduced NT-proBNP levels in the Dahl rat salt-sensitive hypertensive model of HFpEF (FIG. 4).

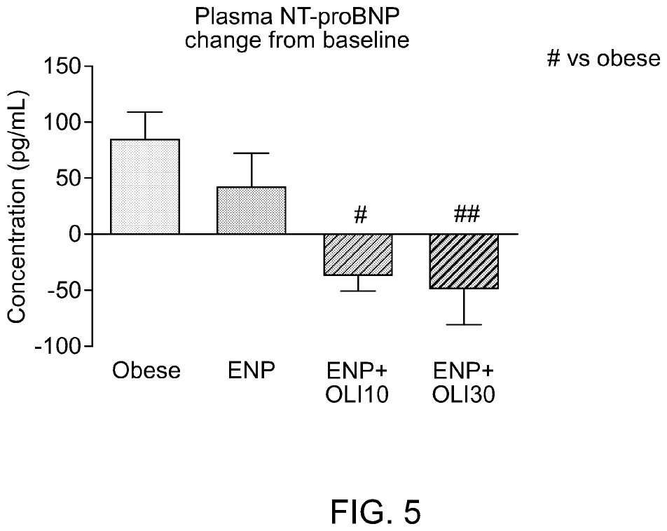

Aged ZSF1 rats have also been reported to develop HF consistent with HFpEF. In the ZSF1 model of diabetic nephropathy and metabolic syndrome, olinciguat was renoprotective and associated with lower circulating glucose, cholesterol, and triglycerides, all of which are relevant for this the population of post-menopausal women with HFpEF that tend to show a metabolic phenotype. Also in this model, baseline plasma NT-proBNP levels were similar in all obese ZSF1 groups [obese, group treated with enalapril (ENP), ENP+OLI10 (enalapril+oli at 10 mg/kg), and ENP+OLI30 (enalapril+oli at 30 mg/kg)]. NT-proBNP, analyzed as change from baseline to the end of treatment, increased in the obese control and ENP groups, and decreased in ENP+OLI10 and ENP+OLI30 groups (FIG. 5). Change from baseline NT-proBNP in the ENP+OLI10 and ENP+OLI30 groups was significantly different from that in the obese control, and there was a trend (p=0.058) toward a difference between ENP+OLI30 and ENP.

1. A method of treating a post-menopausal woman with HFpEF comprising administering to said woman a therapeutically effective amount of an sGC stimulator or pharmaceutically acceptable salt thereof.

2. The method of claim 1, wherein the sGC stimulator is one of Formula I: 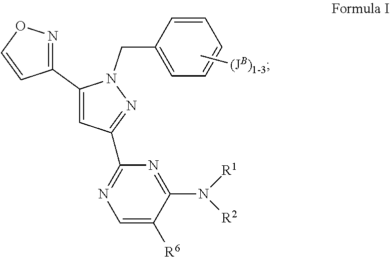
or a pharmaceutically acceptable salt thereof, wherein:
each J^{B} is independently selected from halogen or C₁₋₃ alkyl;
R¹ is hydrogen or C₁₋₃ alkyl;
R² is a C₁₋₆ alkyl group optionally and independently substituted by up to three instances of R⁵,
R⁶ is selected from hydrogen and fluoro; and
each R⁵ is independently selected from hydroxy, C₁₋₃ haloalkyl, halogen and —C(O)NH₂.

3. The method of claim 2, wherein each J^{B} is independently selected from a halogen.

4. The method of claim 3, wherein each J^{B} is fluoro.

5. The method of claim 2, wherein at least one J^{B} is halogen.

6. The method of claim 2, wherein at least one J^{B} is fluoro.

7. The method of claim 2, wherein two J^{B} are halogen; and the third is independently selected from halogen or methyl.

8. The method of any one of claims 2-7, wherein R¹ is hydrogen.

9. The method of any one of claims 2-7, wherein R¹ is methyl or ethyl.

10. The method of claim 9, wherein R¹ is methyl.

11. The method of any one of claims 2-10, wherein R² is C₁₋₃ alkyl independently substituted by up to three instances of R⁵.

12. The method of claim 11, R² is a C₁₋₂ alkyl independently substituted by up to three instances of R⁵.

13. The method of any one of claims 2-12, wherein R⁵ is independently selected from hydroxy, trifluoromethyl or —C(O) NH₂.

14. The method of any one of claims 2-13, wherein R⁶ is hydrogen.

15. The method of any one of claims 2-13, wherein R⁶ is fluoro.

16. The method of claim 2, wherein the sGC stimulator is selected from:

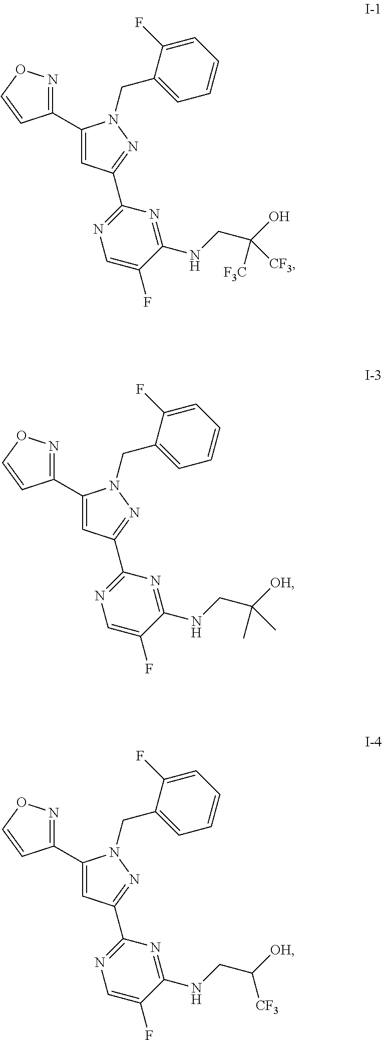 [SMILES: OC(CNc1nc(-c2cc(-c3ccon3)n(Cc3ccccc3F)n2)ncc1F)(C(F)(F)F)C(F)(F)F ; CC(C)(O)CNc1nc(-c2cc(-c3ccon3)n(Cc3ccccc3F)n2)ncc1F ; OC(CNc1nc(-c2cc(-c3ccon3)n(Cc3ccccc3F)n2)ncc1F)C(F)(F)F]

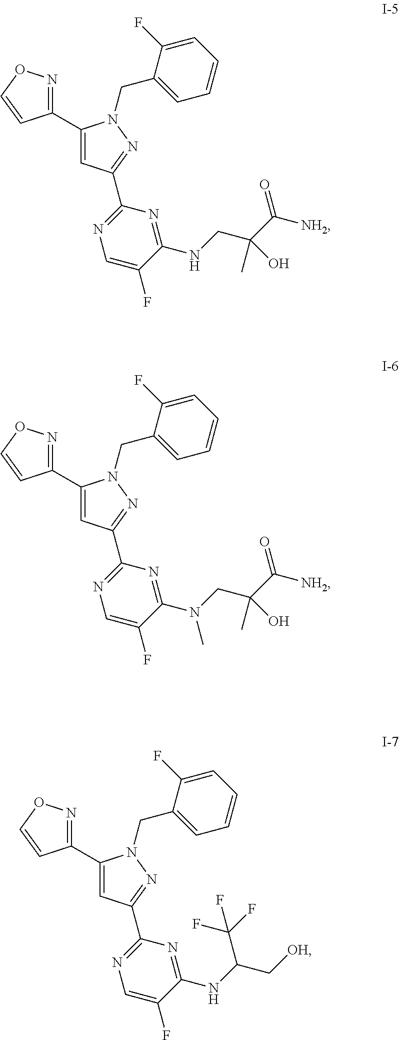 [SMILES: CC(O)(CNc1nc(-c2cc(-c3ccon3)n(Cc3ccccc3F)n2)ncc1F)C(N)=O ; CN(CC(C)(O)C(N)=O)c1nc(-c2cc(-c3ccon3)n(Cc3ccccc3F)n2)ncc1F ; OCC(Nc1nc(-c2cc(-c3ccon3)n(Cc3ccccc3F)n2)ncc1F)C(F)(F)F]

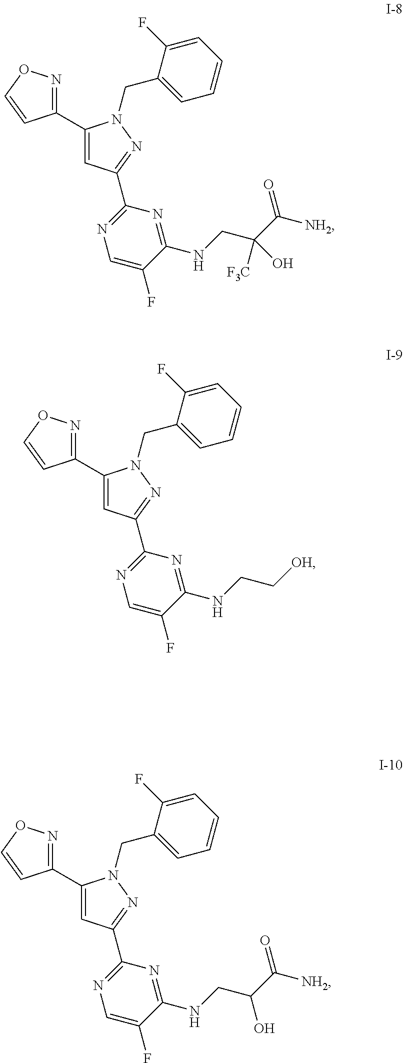 [SMILES: NC(=O)C(O)(CNc1nc(-c2cc(-c3ccon3)n(Cc3ccccc3F)n2)ncc1F)C(F)(F)F ; OCCNc1nc(-c2cc(-c3ccon3)n(Cc3ccccc3F)n2)ncc1F ; NC(=O)C(O)CNc1nc(-c2cc(-c3ccon3)n(Cc3ccccc3F)n2)ncc1F]

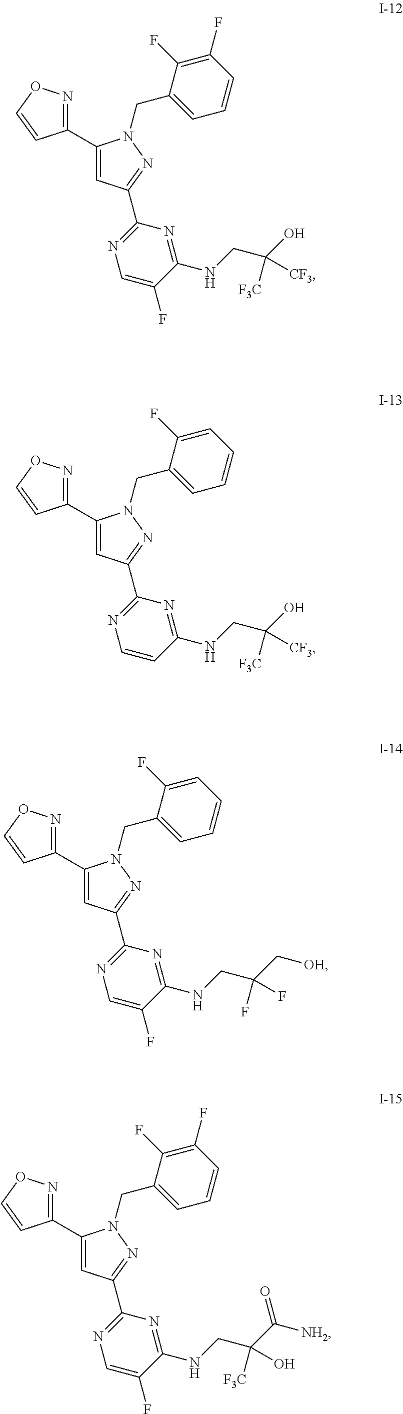 [SMILES: OC(CNc1nc(-c2cc(-c3ccon3)n(Cc3cccc(F)c3F)n2)ncc1F)(C(F)(F)F)C(F)(F)F ; OC(CNc1ccnc(-c2cc(-c3ccon3)n(Cc3ccccc3F)n2)n1)(C(F)(F)F)C(F)(F)F ; OCC(F)(F)CNc1nc(-c2cc(-c3ccon3)n(Cc3ccccc3F)n2)ncc1F ; NC(=O)C(O)(CNc1nc(-c2cc(-c3ccon3)n(Cc3cccc(F)c3F)n2)ncc1F)C(F)(F)F]

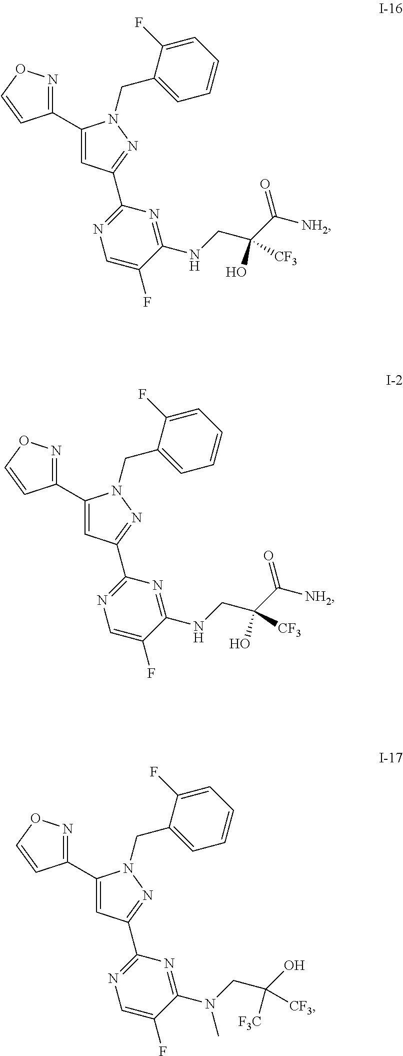 [SMILES: NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F ; CN(CC(O)(C(F)(F)F)C(F)(F)F)c1nc(-c2cc(-c3ccon3)n(Cc3ccccc3F)n2)ncc1F]

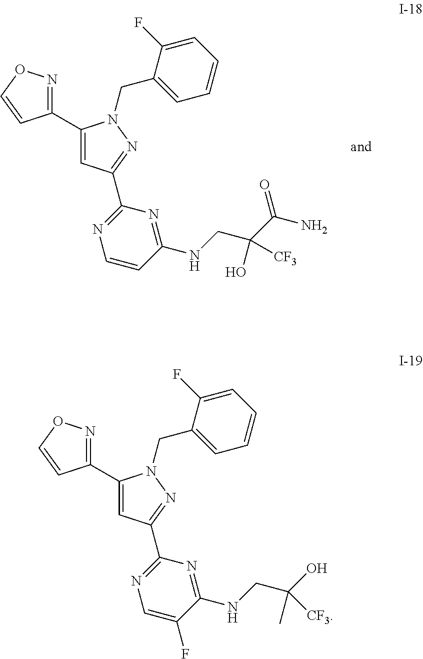 [SMILES: NC(=O)C(O)(CNc1ccnc(-c2cc(-c3ccon3)n(Cc3ccccc3F)n2)n1)C(F)(F)F ; CC(O)(CNc1nc(-c2cc(-c3ccon3)n(Cc3ccccc3F)n2)ncc1F)C(F)(F)F]

17. The method of claim 2, wherein the sGC stimulator is selected from:

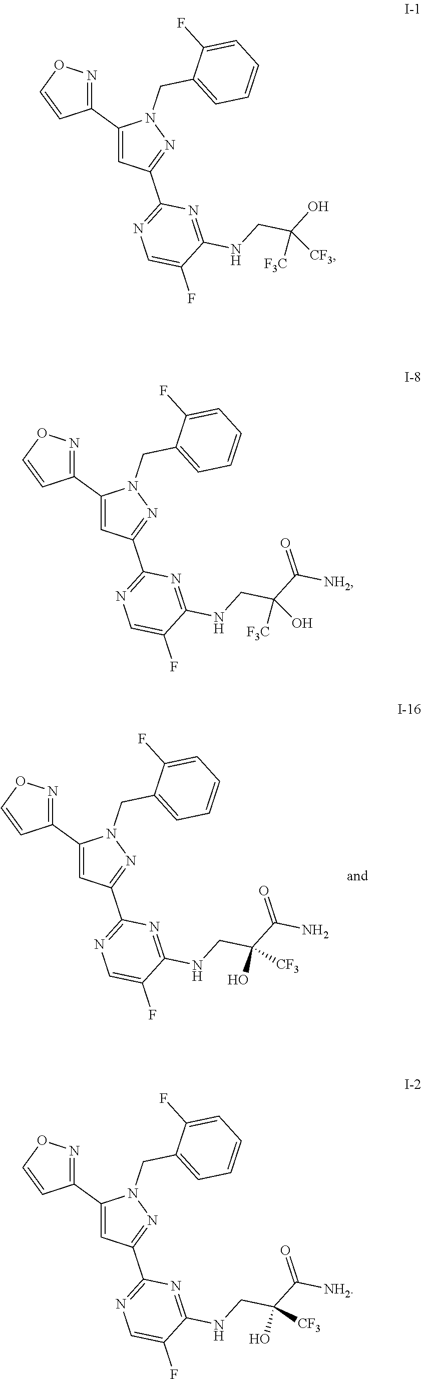 [SMILES: OC(CNc1nc(-c2cc(-c3ccon3)n(Cc3ccccc3F)n2)ncc1F)(C(F)(F)F)C(F)(F)F ; NC(=O)C(O)(CNc1nc(-c2cc(-c3ccon3)n(Cc3ccccc3F)n2)ncc1F)C(F)(F)F ; NC(=O)[C@@](O)(CNc1nc(-c2cc(-c3ccon3)n(Cc3ccccc3F)n2)ncc1F)C(F)(F)F ; NC(=O)[C@](O)(CNc1nc(-c2cc(-c3ccon3)n(Cc3ccccc3F)n2)ncc1F)C(F)(F)F]

18. The method of claim 2, wherein the sGC stimulator is selected from:

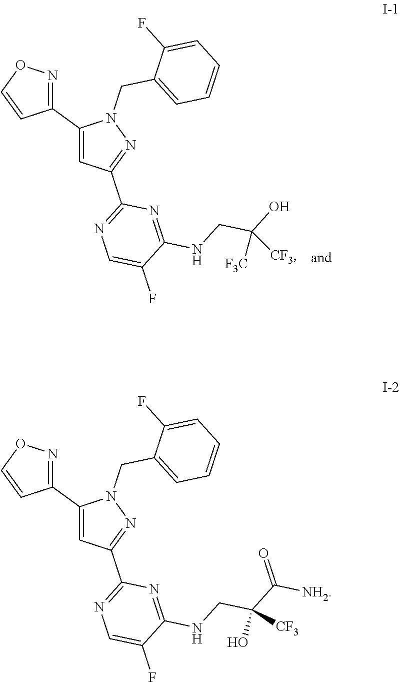 [SMILES: OC(CNc1nc(-c2cc(-c3ccon3)n(Cc3ccccc3F)n2)ncc1F)(C(F)(F)F)C(F)(F)F ; NC(=O)[C@](O)(CNc1nc(-c2cc(-c3ccon3)n(Cc3ccccc3F)n2)ncc1F)C(F)(F)F]

19. The method of claim 2, wherein the sGC stimulator is represented by the following formula:

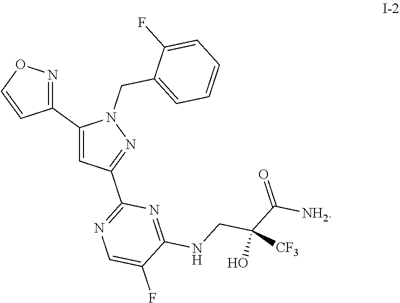 [SMILES: NC(=O)[C@](O)(CNc1nc(-c2cc(-c3ccon3)n(Cc3ccccc3F)n2)ncc1F)C(F)(F)F]

20. The method of claim 1, wherein the sGC stimulator is one of Formula II: 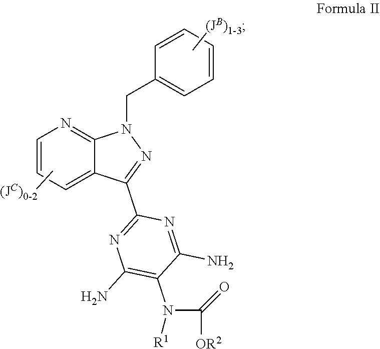
or a pharmaceutically acceptable salt thereof, wherein:
each J^{B} is independently selected from halogen and C₁₋₃ alkyl;
each J^{C}, if present, is independently selected from halogen;
R¹ is hydrogen or C₁₋₃ alkyl; and
R² is a C₁₋₃ alkyl.

21. The method of claim 20, wherein each J^{B} is a fluoro.

22. The method of claim 20, wherein at least one J^{B} is fluoro.

23. The method of claim 20, wherein at least two J^{B} is fluoro.

24. The method of any one of claims 20-23, wherein one or two instances of J^{C} are present.

25. The method of any one of claims 20-23, wherein only one instance of J^{C} is present.

26. The method of any one of claims 20-25, wherein J^{C} is fluoro.

27. The method of any one of claims 20-26, wherein R¹ is hydrogen, methyl or ethyl.

28. The method of claim 27, wherein R¹ is hydrogen.

29. The method of claim 27, wherein R¹ is methyl.

30. The method of any one of claims 20-29, wherein R² is methyl or ethyl.

31. The method of claim 30, wherein R² is methyl.

32. The method of claim 20, wherein the sGC stimulator is selected form the following:

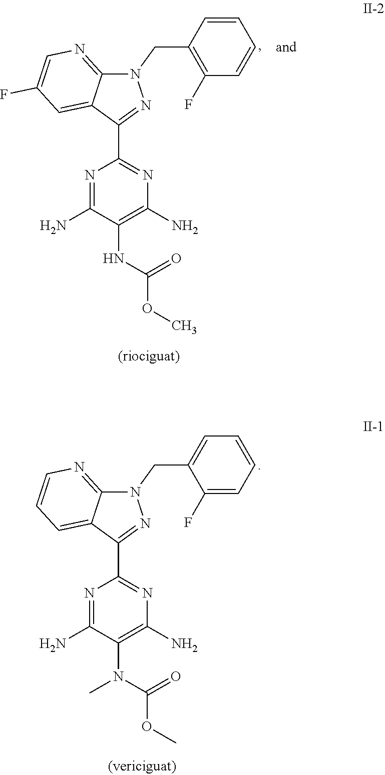 [SMILES: COC(=O)Nc1c(N)nc(-c2nn(Cc3ccccc3F)c3ncc(F)cc23)nc1N ; COC(=O)N(C)c1c(N)nc(-c2nn(Cc3ccccc3F)c3ncccc23)nc1N]

33. The method of claim 20, wherein the sGC stimulator is represented by the following formula:

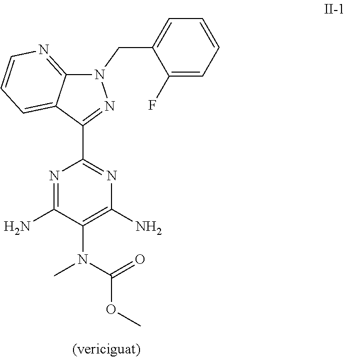 [SMILES: COC(=O)N(C)c1c(N)nc(-c2nn(Cc3ccccc3F)c3ncccc23)nc1N]

34. The method of any one of claims 1-33, wherein the post-menopausal woman is an elderly post-menopausal woman.

35. The method of claim 34, wherein the elderly post-menopausal woman is older than 60 years old, older than 65 years old, older than 70 years old or older than 75 years old.

36. The method of any one of claims 1-35, wherein the post-menopausal woman with HFpEF has established HFpEF.

37. The method of claim 36, wherein the post-menopausal woman with HFpEF has established HF and limited exercise capacity.

38. The method of any one of claims 1-37, wherein the post-menopausal woman has an EF≥40%.

39. The method of any one of claims 1-37, wherein the post-menopausal woman has an EF≥45%.

40. The method of any one of claims 1-39, wherein the post-menopausal woman displays a mean peak VO₂ value ≥60% of the age- and sex-adjusted normal value as measured by CPET.

41. The method of any one of claims 1-39, wherein the post-menopausal woman displays a mean peak VO₂ value <60% of the age- and sex-adjusted normal value as measured by CPET.

42. The method of any one of claims 1-39, wherein the post-menopausal woman displays a mean peak VO₂ value ≥70% of the age- and sex-adjusted normal value as measured by CPET.

43. The method of any one of claims 1-39, wherein the post-menopausal woman displays a mean peak VO₂ value <70% of the age- and sex-adjusted normal value as measured by CPET.

44. The method of any one of claims 1-43, wherein the post-menopausal woman displays at least two out of four conditions selected from: diabetes or prediabetes, hypertension, obesity/overweight and being ≥70 years old.

45. The method of any one of claims 1-43, wherein the post-menopausal woman displays at least two out of four conditions selected from: diabetes or prediabetes, hypertension, obesity/overweight and being ≥60 years old.

46. The method of any one of claims 1-43, wherein the post-menopausal woman displays at least two out of four conditions selected from: diabetes or prediabetes, hypertension, obesity/overweight and being ≥65 years old.

47. The method of any one of claims 1-46, wherein the post-menopausal woman has permanent/persistent atrial fibrillation.

48. The method of any one of claims 1-46, wherein the post-menopausal woman does not have permanent/persistent atrial fibrillation.

49. The method of any one of claims 1-48, wherein the post-menopausal woman has an elevated level of BNP and/or an elevated level of NT-proBNP.

50. The method of any one of claims 1-48, wherein the post-menopausal woman does not have an elevated level of BNP and/or an elevated level of NT-proBNP.

51. The method of any one of claims 1 and 33-50, wherein the post-menopausal woman is administered a single oral daily dose of Compound I-1 of between 10 mg and 40 mg, between 10 mg and 20 mg, between 20 mg and 40 mg, between 20 mg and 30 mg, or between 30 mg and 40 mg.

52. The method of claim 51, wherein the post-menopausal woman is administered a single oral daily dose of 10 mg, 20 mg, 30 mg or 40 mg of Compound I-1.

53. The method of any one of claims 1 and 33-49, wherein the post-menopausal woman is administered an oral dose of 5 mg, 10 mg, 15 mg or 20 mg of Compound I-1 twice per day (BID).

54. The method of any one of claims 1 and 33-49, wherein the post-menopausal woman is administered a single oral daily dose of Compound I-2 of 5 mg and 10 mg, between 5 mg and 15 mg, between 5 mg and 25 mg, between 5 mg and 20 mg, between 10 mg and 15 mg, between 10 mg and 20 mg, between 15 mg and 25 mg, between 15 mg and 20 mg, between 20 mg and 25 mg, between 20 mg and 35 mg, between 25 mg and 35 mg or between 10 mg and 30 mg.

55. The method of claim 54, wherein the post-menopausal woman is administered a single oral daily dose of 6 mg, 9 mg, 12 mg, 15 mg, or 18 mg of Compound I-2.

56. The method of any one of claims 1 and 33-49, wherein the post-menopausal woman is administered a single oral daily dose of Compound II-1 of between 2 mg and 20 mg, between 2.5 mg and 20 mg, between 5 mg and 20 mg, between 5 mg and 15 mg, or between 2.5 mg and 15 mg.

57. The method of claim 56, wherein the post-menopausal woman is administered a single oral daily dose of 2.5 mg, 5 mg, 10 mg or 15 mg of Compound II-1.

58. The method of any one of claims 1-57, wherein the method further comprises administering to the post-menopausal woman one or more anti-hypertensive medications.

59. The method of claim 58, wherein the one or more anti-hypertensive medications are independently selected from an angiotensin-converting enzyme (ACE) inhibitor, an angiotensin II receptor blocker (ARB), a MR antagonist (MRA), an endothelin receptor antagonist (ERA) and a diuretic.

60. The method of claim 58, wherein at least one of the anti-hypertensive medications is a diuretic.

61. The method of claim 58, wherein at least one of the anti-hypertensive medications is an MR antagonist (MRA).

62. The method of claim 58, wherein at least one of the anti-hypertensive medications is an endothelin receptor antagonist (ERA).

63. The method of claim 58, wherein the one or more anti-hypertensive medications are independently selected from an angiotensin-converting enzyme (ACE) inhibitor, an angiotensin II receptor blocker (ARB), and a diuretic.

64. The method of claim 63, wherein the one or more anti-hypertensive medication is independently selected from the group consisting of lisinopril, combinations of lisinopril with hydrochlorothiazide, benazepril, captopril, enalapril, candesartan, losartan, azilsartan, eprosartan, irbesartan, olmesartan, telmisartan, and valsartan.

65. The method of claim 63, wherein the one or more anti-hypertensive medications are each independently selected from the group consisting of lisinopril, combination of lisinopril and hydrochlorothiazide, enalapril, losartan, metoprolol, eplerenone, chlorthalidone, and spironolactone.

66. The method of claim 63, wherein the one or more anti-hypertensive medications are each independently selected from the group consisting of lisinopril, combination of lisinopril and hydrochlorothiazide, enalapril, and losartan.

67. The method of claim 58, wherein at least one of the anti-hypertensive medications is an angiotensin-converting enzyme (ACE) inhibitor or an angiotensin II receptor blocker (ARB).

68. The method of claim 67, wherein at least one of the anti-hypertensive medications is selected from the group consisting of lisinopril, combination of lisinopril and hydrochlorothiazide, enalapril, and losartan.

69. The method of any one of claims 1-68, wherein the method further comprises administering to the post-menopausal woman one or more blood glucose-lowering medications.

70. The method of claim 69, wherein the one or more blood glucose-lowering medications are independently selected from a biguanide, a GLP-1 receptor agonist, a SGLT2 inhibitor (alone or in combination with a SGLT1 inhibitor), and insulin therapy.

71. The method of claim 70, wherein the one or more blood glucose-lowering medications are GLP-1 receptor agonists.

72. The method of claim 69, wherein the one or more blood glucose-lowering medications are independently selected from the group consisting of insulin, metformin, glyburide, glipizide, glimepiride, repaglinide, nateglinide, sitagliptin, saxagliptin, linagliptin, exenatide, liraglutide, semaglutide, empagliflozin, canagliflozin, and dapagliflozin.

73. The method of any one of claims 1-72, wherein the method further comprises administering to the post-menopausal woman one or more anti-hypertensive medications and one or more blood glucose-lowering medications.

74. The method of claim 73, wherein the method further comprises administering to the post-menopausal woman one or more anti-hypertensive medications independently selected from the group consisting of lisinopril, combination of lisinopril and hydrochlorothiazide, enalapril, losartan, metoprolol, and spironolactone and one or more blood glucose-lowering medications independently selected from the group consisting of insulin, metformin, and glipizide.

75. The method of any one of claims 1-74, wherein the method further comprises administering to the post-menopausal woman one or more anti-hyperlipidemic medications.

76. The method of claim 75, wherein the one or more anti-hyperlipidemic medications is a cholesterol-lowering medication.

77. The method of claim 75, wherein the one or more anti-hyperlipidemic medications are independently selected from the group consisting of atorvastin, pravastatin, simvastatin, rosuvastatin, lovastatin, and nicotinic acid.

78. The method of claim 76, wherein the one or more cholesterol-lowering medications is selected from the group consisting of atorvastin, pravastatin, rosuvastatin, lovastatin, and simvastatin.

79. The method of any one of claims 1-78, wherein the method further comprises administering to the patient one or more neprilysin inhibitors.

80. The method of claim 79, wherein the neprilysin inhibitor is sacubitril or the combination of sacubitril with valsartan.

81. The method of any one of claims 1-80, wherein the method further comprises administering to the post-menopausal woman one or more renoprotective medications.

82. The method of claim 81, wherein the renoprotective medications is selected from the group consisting of bardoxolone, irbesartan, losartan, captopril, finerenone, canagliflozin, and atrasentan.

83. The method of any one of claims 1-82, wherein the method further comprises administering to the post-menopausal woman one or more medications for the treatment of pulmonary arterial hypertension (PAH).

84. The method of claim 83, wherein the one or more medications for the treatment of PAH are independently selected from iloprost, ambrisentan, epoprosterol, alprostadil, and trepostinil.

85. The method of any one of claims 1-84, wherein the method further comprises administering to the post-menopausal woman one or more strong cytochrome P450 3A (CYP3A) inhibitors.

86. The method of claim 85, wherein the one or more CYP3A inhibitors is administered to the post-menopausal woman concomitantly with the sGC stimulator.

87. The method of claim 85 or 86, wherein the one or more CYP3A inhibitors are selected from the group consisting of azole antifungals, macrolide antibiotics, protease inhibitors, and diltiazem.

88. The method of any one of claims 1-87, wherein the method improves exercise capacity in the post-menopausal woman.

89. The method of claim 88, wherein the exercise capacity is measured by peak VO₂.

90. The method of any one of claims 1-89, wherein the method improves functional capacity in the post-menopausal woman.

91. The method of claim 90, wherein the functional capacity is measured via the 6-minute walk test (6MWT).

92. The method of any one of claims 1-91, wherein the method improves the heart function in the post-menopausal woman.

93. The method of claim 92, wherein the heart function is measured by electrocardiogram (ECG) and/or echocardiography.

94. The method of any one of claims 1-93, wherein the method improves health status and quality of life in the post-menopausal woman.

95. The method of any one of claims 1-94, wherein the method improves New York Heart Association (NYHA) functional class in the post-menopausal woman.

96. The method of claim 95, wherein the method delays or prevents clinical worsening in the post-menopausal woman by delaying or preventing the patient from worsening in the NYHA functional class scale.

97. The method of any one of claims 1-94, wherein the method reduces the risk of hospitalization due to HF in the post-menopausal woman.

98. The method of any one of claims 1-94, wherein the method reduces the risk of re-hospitalization due to HF in the post-menopausal woman.

99. The method of any one of claims 1-94, wherein the method reduces the risk of cardiovascular death in the post-menopausal woman.

100. The method of any one of claims 1-94, wherein the method reduces the all-cause mortality risk.
